(19) United States
(12) Patent Application Publication
(10) Pub. No.: US 2004/0067992 A1
(43) Pub. Date: Apr. 8, 2004

(54) Compositions of a cyclooxygenase-2 selective inhibitor and a carbonic anhydrase inhibitor for the treatment of neoplasia

(75) Inventors: Jaime L. Masferrer, Ballwin, MO (US); Janet M. O'Neal, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/366,739

(22) Filed: Feb. 14, 2003

Related U.S. Application Data
(63) Continuation-in-part of application No. 10/213,793, filed on Aug. 7, 2002.
(60) Provisional application No. 60/311,561, filed on Aug. 10, 2001.

(51) Int. Cl. A61K 31/428; A61K 31/415

(52) U.S. Cl. 514/369; 514/406

(57) ABSTRACT

The present invention provides compositions and methods for the treatment of neoplasia in a subject. More particularly, the invention provides a combination therapy for the treatment of neoplasia comprising the administration to a subject of a carbonic anhydrase inhibitor in combination with a cyclooxygenase-2 selective inhibitor.

### CROSS REFERENCE TO RELATED APPLICATIONS

This application is a continuation in part of application Serial No. 10/213,793 filed on Aug. 7, 2002, which claims priority from provisional application Serial No. 60/311,561 filed on Aug. 10, 2001, both of which are hereby incorporated by reference in their entirety.

### FIELD OF THE INVENTION

The present invention provides compositions and methods for the treatment of a neoplasia. More particularly, the invention is directed toward a combination therapy for the treatment or prevention of neoplasia comprising the administration to a subject of a carbonic anhydrase inhibitor in combination with a cyclooxygenase-2 selective inhibitor.

### BACKGROUND OF THE INVENTION

Currently, non-surgical cancer treatment regimes involve administering one or more highly toxic chemotherapeutics or hormonal therapies to the patient after the cancer has progressed to a point where the therapeutic benefits of chemotherapy/hormonal outweigh its serious side effects. As a consequence of these side effects, standard chemotherapeutics are typically used only for short periods of time, often alternating chemotherapy with periods off treatment, so as not to overwhelm the patient with drug side effects. Accordingly, given the risk-benefit trade-off, side effects typically preclude starting chemotherapy when patients exhibit precancerous lesions, or continuing chemotherapy or hormonal therapy on a chronic basis after cancer has been eliminated in an attempt to prevent its re-occurrence.

Cancer and precancer research is replete with publications that describe various biochemical molecules that are over-expressed in neoplastic tissue, leading several groups to research whether specific over-expressed molecules are responsible for the disease, and whether, if such over-expression were inhibited, neoplasia could be alleviated. One such biochemical molecule that has been extensively studied as a therapeutic target for neoplasia treatment is the prostaglandins, which are naturally occurring C-20 unsaturated fatty acids. By way of example, in familial adenomatous polyposis (“FAP”), Waddell et al. hypothesized that since prostaglandins were over-expressed in such polyps, non-steroidal anti-inflammatory drugs (“NSAIDs”) should alleviate the condition because NSAIDs inhibited prostaglandin synthesis. Thus, he administered the NSAID sulindac (an inhibitor of PGE₂) to several FAP patients. Waddell et al. discovered that polyps regressed and did not recur upon therapeutic treatment with an NSAID. PGE₂ inhibition results from the inhibition of cyclooxygenase (COX) by NSAIDs.

While patients treated with NSAIDS typically exhibit far fewer side effects than with conventional chemotherapeutics or hormonals, the use of high doses of most common NSAIDs can produce severe side effects, including life-threatening ulcers that limit their therapeutic potential. One reason proposed for the severe side effects associated with traditional NSAIDs is their non-selective inhibition of both of the cyclooxygenase enzymes (COX), commonly known as COX-1 and COX-2. COX-1 is constitutively expressed and mediates a number of physiological functions, such as kidney and gastrointestinal function. COX-2 expression, contrastingly, is stimulated by a number of inflammatory cytokines, growth factors, oncogenes, lipopolysaccharides, and tumor promoters. While conventional NSAIDs block both forms of the enzyme, a new class of NSAID, selective cyclooxygenase-2 inhibitors, provide a viable target of inhibition that more effectively reduces inflammation and produces fewer and less drastic side effects.

COX-2 plays a key role in tumorigenesis through stimulating epithelial cell proliferation, inhibiting apoptosis, stimulating angiogenesis, enhancing cell invasiveness, mediating immune suppression, and by increasing the production of mutagens. Results of several studies using mouse models of colon cancer and the results of clinical trials have shown COX-2 to be a useful target for the prevention and treatment of colon cancer (Fernandex et al., (2002) In Vivo 16(6):501-509). Studies with several other epithelial cancers involving different organ sites, e.g., breast, prostate, bladder, lung, and pancreas, suggest that COX-2 plays an important role in the pathogenesis of these cancers (e.g. for its role in breast cancer see Singh et al., (2002) J. Surg. Res. 108(1):173-179; for its role in fibroblasts and endothelial cells see Sonoshita et al., (2002) Cancer Res. 62(23):6846-6849; for its role in gastric cells see Li et al., (2002) 21(6):625-629).

### SUMMARY OF THE INVENTION

Among the several aspects of the invention is provided a method and a composition for the treatment of neoplasia in a subject. The composition comprises a cyclooxygenase-2 selective inhibitor or pharmaceutically acceptable salt or prodrug thereof and a carbonic anhydrase inhibitor or pharmaceutically acceptable salt or prodrug thereof. In another aspect, the method comprises administering to the subject a cyclooxygenase-2 selective inhibitor or pharmaceutically acceptable salt or prodrug thereof in combination with a carbonic anhydrase inhibitor or pharmaceutically acceptable salt or prodrug thereof.

In one embodiment, the cyclooxygenase-2 selective inhibitor is a member of the chromene class of compounds. For example, the chromene compound or pharmaceutically acceptable salt or prodrug thereof may be a compound of the formula:

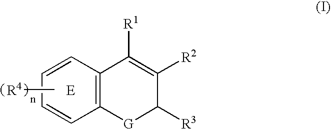

wherein:

n is an integer which is 0, 1, 2, 3 or 4;

G is O, S or NR^{a};

R^{a} is alkyl;

R¹ is selected from the group consisting of H and aryl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl optionally substituted with one or more radicals selected from alkylthio, nitro and alkylsulfonyl; and

each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

In another embodiment, the cyclooxygenase-2 selective inhibitor or pharmaceutically acceptable salt or prodrug thereof comprises a compound of the formula:

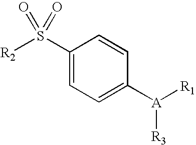

wherein

A is selected from the group consisting of partially unsaturated or unsaturated heterocyclyl and partially unsaturated or unsaturated carbocyclic rings;

R¹ is selected from the group consisting of heterocyclyl, cycloalkyl, cycloalkenyl and aryl, wherein R1 is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is selected from the group consisting of methyl or amino; and

R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In yet another embodiment, the carbonic anhydrase inhibitor is dorzolamide. In another embodiment, the carbonic anhydrase inhibitor is acetazolamide. In still another embodiment, the carbonic anhydrase inhibitor is dichlorophenamide. In yet a further embodiment, the carbonic anhydrase inhibitor is brinzolamide. In another embodiment, the carbonic anhydrase inhibitor is methazolamide.

Other aspects of the invention are described in more detail below.

Abbreviations and Definitions

The term “acyl” is a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl.

The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

Where used, either alone or within other terms such as “haloalkyl”, “alkylsulfonyl”, “alkoxyalkyl” and “hydroxyalkyl”, the term “alkyl” is a linear, cyclic or branched radical having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkylamino” is an amino group that has been substituted with one or two alkyl radicals. Preferred is “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like.

The term “alkylaminoalkyl” is a radical having one or more alkyl radicals attached to an aminoalkyl radical.

The term “alkylaminocarbonyl” is an aminocarbonyl group that has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “alkylthio” is a radical containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio.

The term “alkylthioalkyl” is a radical containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” is a radical containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “alkynyl” is a linear or branched radical having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The term “aminoalkyl” is an alkyl radical substituted with one or more amino radicals. More preferred are “lower aminoalkyl” radicals. Examples of such radicals include aminomethyl, aminoethyl, and the like.

The term “aminocarbonyl” is an amide group of the formula —C(═O)NH2.

The term “aralkoxy” is an aralkyl radical attached through an oxygen atom to other radicals.

The term “aralkoxyalkyl” is an aralkoxy radical attached through an oxygen atom to an alkyl radical.

The term “aralkyl” is an aryl-substituted alkyl radical such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable.

The term “aralkylamino” is an aralkyl radical attached through an amino nitrogen atom to other radicals. The terms “N-arylaminoalkyl” and “N-aryl-N-alkyl-aminoalkyl” are amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aralkylthio” is an aralkyl radical attached to a sulfur atom.

The term “aralkylthioalkyl” is an aralkylthio radical attached through a sulfur atom to an alkyl radical.

The term “aroyl” is an aryl radical with a carbonyl radical as defined above. Examples of aroyl include benzoyl, naphthoyl, and the like and the aryl in said aroyl may be additionally substituted.

The term “aryl”, alone or in combination, means a carbocyclic aromatic system containing one, two or three rings wherein such rings may be attached together in a pendent manner or may be fused. The term “aryl” is an aromatic radical such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “arylamino” is an amino group, which has been substituted with one or two aryl radicals, such as N-phenylamino. The “arylamino” radicals may be further substituted on the aryl ring portion of the radical.

The term “aryloxyalkyl” is a radical having an aryl radical attached to an alkyl radical through a divalent oxygen atom.

The term “arylthioalkyl” is a radical having an aryl radical attached to an alkyl radical through a divalent sulfur atom.

The term “carbonic anhydrase” as used herein refers to any isomer of the metalloprotein enzyme that catalyzes the interconversion of CO₂ and H₂CO₃(CO₂+O₂→HCO₂⁻+H⁺).

The term “carbonyl”, whether used alone or with other terms, such as “alkoxycarbonyl”, is —(C═O)—.

The terms “carboxy” or “carboxyl”, whether used alone or with other terms, such as “carboxyalkyl”, is —CO2H.

The term “carboxyalkyl” is an alkyl radical substituted with a carboxy radical. More preferred are “lower carboxyalkyl” which are lower alkyl radicals as defined above, and may be additionally substituted on the alkyl radical with halo. Examples of such lower carboxyalkyl radicals include carboxymethyl, carboxyethyl and carboxypropyl.

The term “cycloalkenyl” is a partially unsaturated carbocyclic radical having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl.

The term “cyclooxygenase-2 selective inhibitor” is a compound able to inhibit cyclooxygenase-2 without significant inhibition of cyclooxygenase-1. Preferably, it includes compounds that have a cyclooxygenase-2 IC₅₀ of less than about 0.2 micro molar, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 micro molar, and more preferably of greater than 10 micro molar. Inhibitors of the cyclooxygenase pathway in the metabolism of arachidonic acid used in the present method may inhibit enzyme activity through a variety of mechanisms. By the way of example, and without limitation, the inhibitors used in the methods described herein may block the enzyme activity directly by acting as a substrate for the enzyme.

The term “halo” means halogens such as fluorine, chlorine, bromine or iodine.

The term “haloalkyl” is a radical wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” are radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl” is an unsaturated heterocyclyl radical. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino.

The term “heterocyclyl” is a saturated, partially unsaturated and unsaturated heteroatom-containing ring-shaped radical, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole.

The term “heterocyclylalkyl” is a saturated and partially unsaturated heterocyclyl-substituted alkyl radical, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy.

The term “hydrido” is a single hydrogen atom (H). This hydrido radical may be attached, for example, to an oxygen atom to form a hydroxyl radical or two hydrido radicals may be attached to a carbon atom to form a methylene (—CH2-) radical.

The term “hydroxyalkyl” is a linear or branched alkyl radical having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The term “inhibition” as used herein means to decrease the severity of neoplasia or a neoplasia disorder as compared to that which would occur in the absence of the administration of a compound identified herein as either a COX-2 selective inhibitor or carbonic anhydrase inhibitor.

The term “inhibitor” when used herein unless otherwise indicated refers to an enzyme inhibitor such as an inhibitor of carbonic anhydrase or cyclooxygenase. Enzyme inhibitors are agents and/or compounds that stop, prevent, or reduce the rate of an enzymatic reaction via any mechanism including, but not limited to, competitive inhibition, noncompetitive inhibition, and uncompetitive inhibition.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzyl ethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

The term “prevention” includes either preventing the onset of clinically evident neoplasia altogether or preventing the onset of a preclinically evident stage of neoplasia in individuals at risk. Also encompassed by this definition is the prevention of initiation for malignant cells or to arrest or reverse the progression of premalignant cells to malignant cells. This includes prophylactic treatment of those at risk of developing the neoplasia.

The term “prodrug” refers to a chemical compound that can be converted into a therapeutic compound by metabolic or simple chemical processes within the body of the subject. For example, a class of prodrugs of COX-2 inhibitors is described in U.S. Pat. No. 5,932,598, herein incorporated by reference.

The term “subject” for purposes of treatment includes any human or animal subject who is susceptible to an adverese impact resulting from a decrease in blood flow to the central nervous system. The subject can be a domestic livestock species, a laboratory animal species, a zoo animal or a companion animal. In one embodiment, the subject is a mammal. In another embodiment, the mammal is a human being.

The term “sulfonyl”, whether used alone or linked to other terms such as alkylsulfonyl, is divalent radicals —SO₂—. “Alkylsulfonyl” are alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” are NH₂O₂S—.

The phrase “therapeutically-effective” is intended to qualify the amount of each agent (i.e. the amount of cyclooxygenase-2 selective inhibitor and the amount of carbonic anhydrase inhibitor) which will achieve the goal of improvement in disorder severity and the frequency of incidence over no treatment or treatment of each agent by itself.

The term “treatment” includes partial or total inhibition of the neoplasia growth, spreading or metastasis, as well as partial or total destruction of the neoplasia cells. Treatment also includes prevention of a neoplasia or related disorder.

### DESCRIPTION OF THE PREFERRED EMBODIMENTS

The present invention provides a combination therapy comprising the administration to a subject of a therapeutically effective amount of a COX-2 selective inhibitor in combination with a therapeutically effective amount of a second compound that is a carbonic anhydrase inhibitor. The combination therapy may be employed to treat or prevent neoplasia or a neoplasia related disorder. When administered as part of a combination therapy, the COX-2 selective inhibitor together with the carbonic anhydrase inhibitor provide enhanced treatment options as compared to administration of either the carbonic anhydrase inhibitor or the COX-2 selective inhibitor alone.

Cyclooxygenase-2 Selective Inhibitors

A number of suitable cyclooxygenase-2 selective inhibitors or pharmaceutically acceptable salts or prodrugs may be employed in the composition of the current invention. In one embodiment, the cyclooxygenase-2 selective inhibitor can be, for example, the cyclooxygenase-2 selective inhibitor meloxicam, Formula B-1 (CAS registry number 71125-38-7) or pharmaceutically acceptable salt or prodrug thereof.

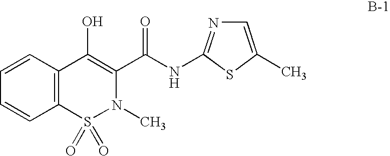

In yet another embodiment, the cyclooxygenase-2 selective inhibitor is the cyclooxygenase-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or pharmaceutically acceptable salt or prodrug thereof.

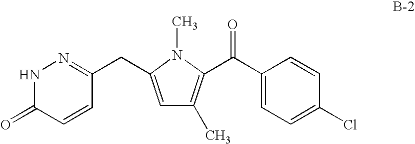

In yet another embodiment the cyclooxygenase-2 selective inhibitor is a chromene compound that is a substituted benzopyran or a substituted benzopyran analog, and even more typically, selected from the group consisting of substituted benzothiopyrans, dihydroquinolines, dihydronaphthalenes or a compound having Formula I shown below and possessing, by way of example and not limitation, the structures disclosed in Table 1x. Furthermore, benzopyran cyclooxygenase-2 selective inhibitors useful in the practice of the present methods are described in U.S. Pat. Nos. 6,034,256 and 6,077,850 herein incorporated by reference in their entirety.

In one embodiment, the cyclooxygenase-2 selective inhibitor or pharmaceutically acceptable salt or prodrug thereof is a chromene compound represented by Formula I:

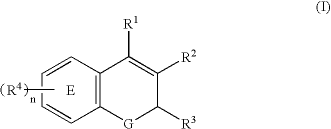

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

The cyclooxygenase-2 selective inhibitor may also be a compound of Formula (I) or pharmaceutically acceptable salt or prodrug thereof wherein:

n is an integer which is 0, 1, 2, 3 or 4;

G is O, S or NR^{a};

R¹ is H;

R^{a} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

In a further embodiment, the cyclooxygenase-2 selective inhibitor may also be a compound of Formula (I), or pharmaceutically acceptable salt or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (J) or pharmaceutically acceptable salt or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or pharmaceutically acceptable salt or prodrug thereof; wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or pharmaceutically acceptable salt or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

In yet another embodiment, the cyclooxygenase-2 selective inhibitor used in connection with the method(s) of the present invention can also be a compound having the structure of Formula (I) or pharmaccutically acceptable salt or prodrug thereof:

wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R¹¹ corresponding to R¹² is H, halo, lower alkyl, lower alkoxy, and aryl;

wherein Formula (I) is represented by Formula (Ia):

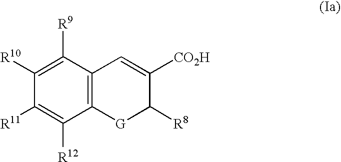

The cyclooxygenase-2 selective inhibitor used in connection with the method(s) of the present invention can also be a compound of having the structure of Formula (Ia) or pharmaceutically acceptable salt or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

Examples of exemplary chromene cyclooxygenase-2 selective inhibitors are depicted in Table 1x below.

TABLE 1x
Examples of Chromene Cyclooxygenase-2 Selective
Inhibitors as Embodiments

| Compound | |
|---|---|
| Number | Structural Formula |
| B-3 | 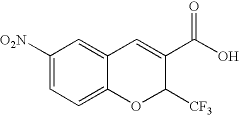 |
| | 6-Nitro-2-trifluoromethyl-2H-1- |
| | benzopyran-3-carboxylic acid |
| B-4 | 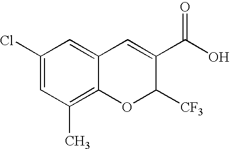 |
| | 6-Chloro-8-methyl-2-trifluoromethyl- |
| | 2H-1-benzopyran-3-carboxylic acid |
| B-5 | 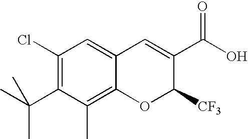 |
| | ((S)-6-Chloro-7-(1,1-dimethylethyl)-2- |
| | (trifluoromethyl-2H-1-benzopyran-3-carboxyic acid |
| B-6 | 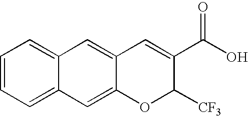 |
| | 2-Trifluoromethyl-2H-naphtho[2,3-b] |
| | pyran-3-carboxylic acid |
| B-7 | 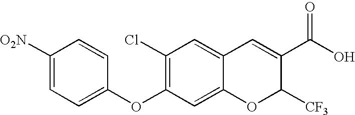 |
| | 6-Chloro-7-(4-nitrophenoxy)-2-(trifluoromethyl)-2H- |
| | 1-benzopyran-3-carboxylic acid |
| B-8 | 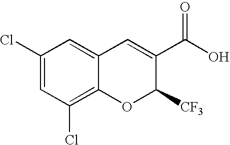 |
| | ((S)-6,8-Dichloro-2-(trifluoromethyl)- |
| | 2H-1-benzopyran-3-carboxylic acid |
| B-9 | 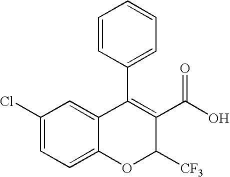 |
| | 6-Chloro-2-(trifluoromethyl)-4-phenyl-2H- |
| | 1-benzopyran-3-carboxylic acid |
| B-10 | 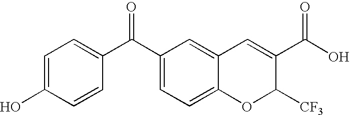 |
| | 6-(4-Hydroxybenzoyl)-2-(trifluoromethyl)- |
| | 2H-1-benzopyran-3-carboxylic acid |
| B-11 | 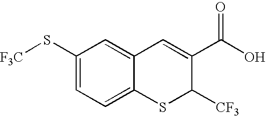 |
| | 2-(Trifluoromethyl)-6-[(trifluoromethyl)thio]- |
| | 2H-1-benzothiopyran-3-carboxylic acid |
| B-12 | 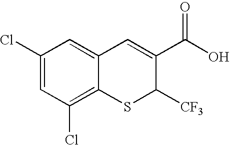 |
| | 6,8-Dichloro-2-trifluoromethyl-2H-1- |
| | benzothiopyran-3-carboxylic acid |
| B-13 | 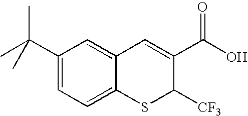 |
| | 6-(1,1-Dimethylethyl)-2-(trifluoromethyl)- |
| | 2H-1-benzothiopyran-3-carboxylic acid |
| B-14 | 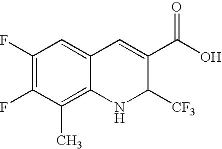 |
| | 6,7-Difluoro-1,2-dihydro-2-(trifluoromethyl)-3- |
| | quinolinecarboxylic acid |
| B-15 | 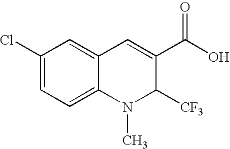 |
| | 6-Chloro-1,2-dihydro-1-methyl-2-(trifluoromethyl)- |
| | 3-quinolinecarboxylic acid |
| B-16 | 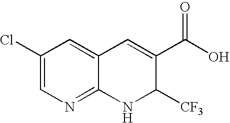 |
| | 6-Chloro-2-(trifluoromethyl)-1,2-dihydro |
| | [1,8]naphthyridine-3-carboxylic acid |
| B-17 | 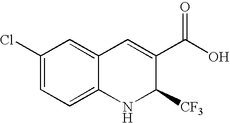 |
| | ((S)-6-Chloro-1,2-dihydro-2-(trifluoromethyl)- |
| | 3-quinolinecarboxylic acid |

In a further embodiment, the cyclooxygenase-2 selective inhibitor or pharmaceutically acceptable salt or prodrug thereof is selected from the class of tricyclic cyclooxygenase-2 selective inhibitors represented by the general structure of Formula II:

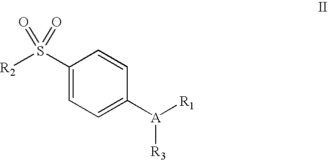

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

In another embodiment, the cyclooxygenase-2 selective inhibitor or pharmaceutically acceptable salt or prodrug thereof represented by the above Formula II is selected from the group of compounds, illustrated in Table 2x, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23), or pharmaceutically acceptable salt or prodrug thereof.

TABLE 2x
Examples of Tricyclic Cyclooxygenase-2 Selective
Inhibitors as Embodiments

| Compound | |
|---|---|
| Number | Structural Formula |
| B-18 | 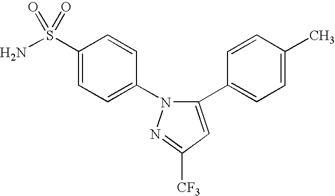 |
| B-19 | 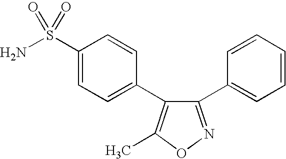 |
| B-20 | 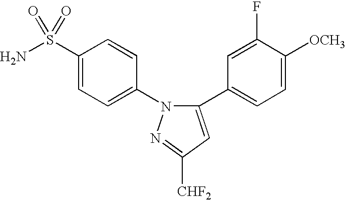 |
| B-21 | 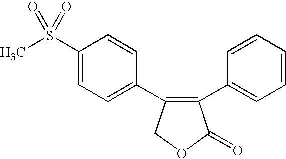 |
| B-22 | 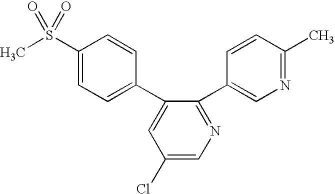 |
| B-23 | 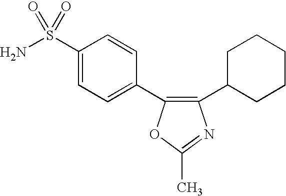 |

In still another embodiment, the cyclooxygenase-2 selective inhibitor is selected from the group consisting of celecoxib, rofecoxib and etoricoxib.

In yet another embodiment, the cyclooxygenase-2 selective inhibitor is parecoxib (B-24, U.S. Pat. No. 5,932,598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

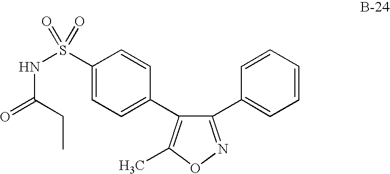

One form of parecoxib is sodium parecoxib.

In another preferred embodiment of the invention, the compound having the formula B-25 that has been previously described in International Publication number WO 00/24719 (which is herein incorporated by reference) is another tricyclic cyclooxygenase-2 selective inhibitor which may be advantageously employed.

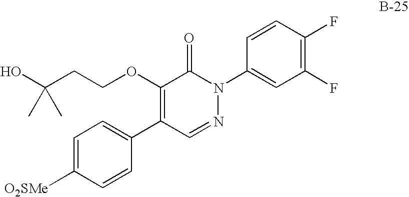

Another cyclooxygenase-2 selective inhibitor that is useful in connection with the method(s) of the present invention is N-(2-cyclohexyloxynitrophenyl)-methane sulfonamide (NS-398) having a structure shown below as B-26.

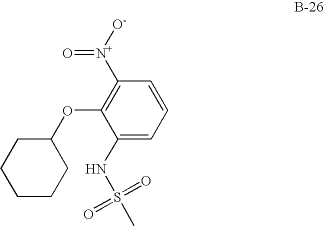

In yet a further embodiment, the cyclooxygenase-2 selective inhibitor or pharmaceutically acceptable salt or prodrug thereof used in connection with the method(s) of the present invention can be selected from the class of phenylacetic acid derivative cyclooxygenase-2 selective inhibitors represented by the general structure of Formula (III):

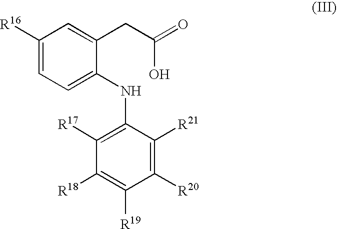

wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

Another phenylacetic acid derivative cyclooxygenase-2 selective inhibitor used in connection with the method(s) of the present invention is a compound that has the designation of COX 189 (B-211) and that has the structure shown in Formula (III) or pharmaceutically acceptable salt or prodrug thereof, wherein:

R¹⁶ is ethyl;

R¹⁷ and R¹⁹ are chloro;

R¹⁸ and R²⁰ are hydrogen; and

and R²¹ is methyl.

In yet another embodiment, the cyclooxygenase-2 selective inhibitor or pharmaceutically acceptable salt or prodrug thereof is represented by Formula (IV):

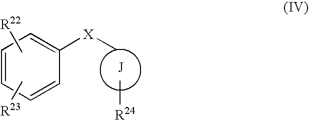

wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the cyclooxygenase-2 selective inhibitors used in the present method(s) have the structural Formula (V):

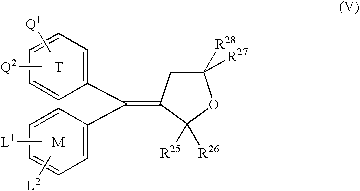

or pharmaceutically acceptable salt or prodrug thereof, wherein: T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, the compounds N-(2-cyclohexyloxynitrophenyl)methane sulfonamide, and (E)-4-[(4-methylphenyl)(tetrahydro-2-oxo-3-furanylidene) methyl] benzenesulfonamide having the structure of Formula (V) are employed as cyclooxygenase-2 selective inhibitors.

In a further embodiment, compounds that are useful for the cyclooxygenase-2 selective inhibitor or pharmaceutically acceptable salt or prodrug thereof in connection with the method(s) of the present invention, the structures for which are set forth in Table 3x below, include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H-1-benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino] sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101); 4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3-chloro-4-fluorophenyl)-4-(4-fluorophenyl)-5-(4-methylsulfonylphenyl)thiazole (B-111);

2-(2-chlorophenyl)-4-(4-fluorophenyl)-5-(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1-dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1-[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3-(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR, 10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231);

S-2474 (B-232);

4-[4-(methyl)-sulfonyl)phenyl]-3-phenyl-2(5H)-furanone;

4-(5-methyl-3-phenyl-4-isoxazolyl);

2-(6-methylpyrid-3-yl)-3-(4-methylsulfonylphenyl)-5-chloropyridine;

4-[5-(4-methylphenyl)-3-(trifluoromethyl)-1H-pyrazol-1-yl];

N-[[4-(5-methyl-3-phenyl-4-isoxazolyl)phenyl]sulfonyl];

4-[5-(3-fluoro-4-methoxyphenyl)-3-difluoromethyl)-1H-pyrazol-1-yl]benzenesulfonamide;

(S)-6,8-dichloro-2-(trifluoromethyl)-2H-1-benzopyran-3-carboxylic acid;

2-(3,4-difluorophenyl)-4-(3-hydroxy-3-methylbutoxy)-5-[4-(methylsulfonyl)phenyl]-3(2H)-pyridzainone;

2-trifluoromethyl-3H-naptho[2,1-b]pyran-3-carboxylic acid;

6-chloro-7-(1,1-dimethylethyl)-2-trifluoromethyl-2H-1-benzopyran-3-carboxylic acid;

[2-(2,4-dichloro-6-ethyl-3,5-dimethyl-phenylamino)-5-propyl-phenyl]-acetic acid.

TABLE 3x
Examples of Cyclooxygenase-2 Selective Inhibitors as Embodiments

| Compound Number | Structural Formula |
|---|---|
| B-26 | 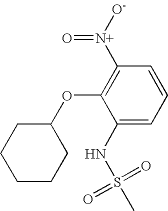 |
| | N-(2-cyclohexyloxynitrophenyl) methane sulfonamide or NS-398; |
| B-27 | 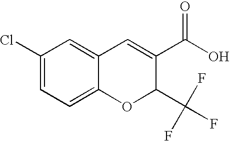 |
| | 6-chloro-2-trifluoromethyl-2H-1-benzopyran-3-carboxylic acid; |
| B-28 | 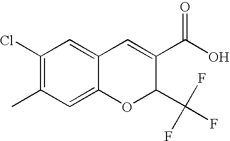 |
| | 6-chloro-7-methyl-2-trifluoromethyl-2H-1-benzopyran-3- |
| | carboxylic acid; |
| B-29 | 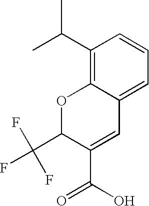 |
| | 8-(1-methylethyl)-2-trifluoromethyl-2H-1-benzopyran-3- |
| | carboxylic acid; |
| B-30 | 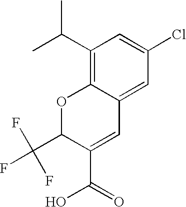 |
| | 6-chloro-8-(1-methylethyl)-2-trifluoromethyl- |
| | 2H-1-benzopyran-3-carboxylic acid; |
| B-31 | 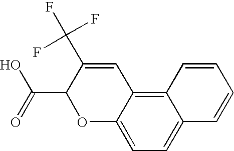 |
| | 2-trifluoromethyl-3H-naphtho]2,1-b]pyran-3- |
| | carboxylic acid; |
| B-32 | 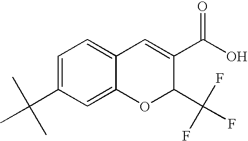 |
| | 7-(1,1-dimethylethyl)-2-trifluoromethyl-2H-1- |
| | benzopyran-3-carboxylic acid; |
| B-33 | 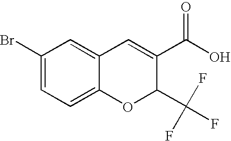 |
| | 6-bromo-2-trifluoromethyl-2H-1-benzopyran-3- |
| | carboxylic acid; |
| B-34 | 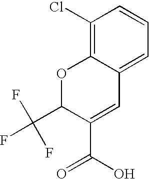 |
| | 8-chloro-2-trifluoromethyl-2H-1-benzopyran-3- |
| | carboxylic acid; |
| B-35 | 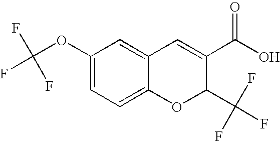 |
| | 6-trifluoromethoxy-2-trifluoromethyl-2H-1- |
| | benzopyran-3-carboxylic acid; |
| B-36 | 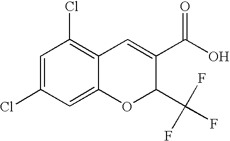 |
| | 5,7-dichloro-2-trifluoromethyl-2H-1-benzopyran- |
| | 3-carboxylic acid; |
| B-37 | 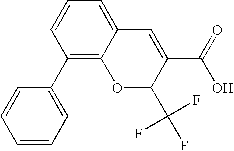 |
| | 8-phenyl-2-trifluoromethyl-2H-1-benzopyran-3- |
| | carboxylic acid; |
| B-38 | 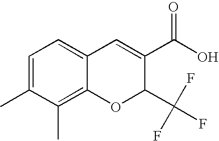 |
| | 7,8-dimethyl-2-trifluoromethyl-2H-1-benzopyran-3- |
| | carboxylic acid; |
| B-39 | 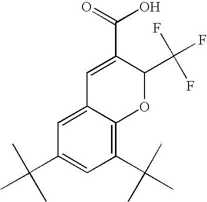 |
| | 6,8-bis(dimethylethyl)-2-trifluoromethyl-2H-1- |
| | benzopyran-3-carboxylic acid; |
| B-40 | 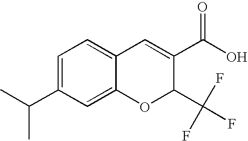 |
| | 7-(1-methylethyl)-2-trifluoromethyl-2H-1-benzopyran- |
| | 3-carboxylic acid; |
| B-41 | 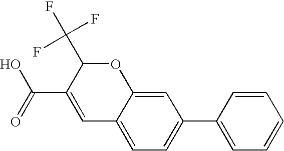 |
| | 7-phenyl-2-trifluoromethyl-2H-1-benzopyran-3- |
| | carboxylic acid; |
| B-42 | 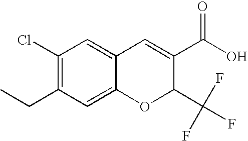 |
| | 6-chloro-7-ethyl-2-trifluoromethyl-2H-1- |
| | benzopyran-3-carboxylic acid; |
| B-43 | 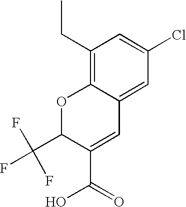 |
| | 6-chloro-8-ethyl-2-trifluoromethyl-2H-1-benzopyran-3- |
| | carboxylic acid; |
| B-44 | 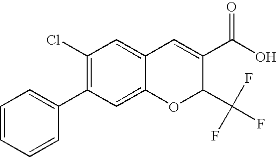 |
| | 6-chloro-7-phenyl-2-trifluoromethyl-2H-1-benzopyran-3- |
| | carboxylic acid; |
| B-45 | 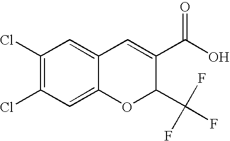 |
| | 6,7-dichloro-2-trifluoromethyl-2H-1-benzopyran-3- |
| | carboxylic acid; |
| B-46 | 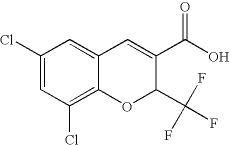 |
| | 6,8-dichloro-2-trifluoromethyl-2H-1-benzopyran-3- |
| | carboxylic acid; |
| B-47 | 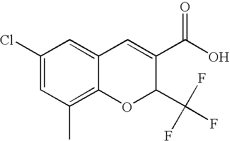 |
| | 6-chloro-8-methyl-2-trifluoromethyl-2H-1-benzopyran- |
| | 3-carboxylic acid; |
| B-48 | 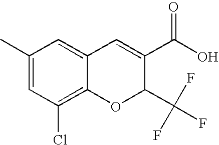 |
| | 8-chloro-6-methyl-2-trifluoromethyl-2H-1-benzopyran-3- |
| | carboxylic acid; |
| B-49 | 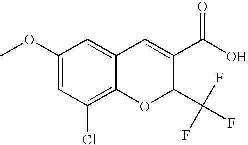 |
| | 8-chloro-6-methoxy-2-trifluoromethyl-2H-1-benzopyran- |
| | 3-carboxylic acid; |
| B-50 | 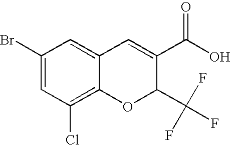 |
| | 6-bromo-8-chloro-2-trifluoromethyl-2H-1-benzopyran-3- |
| | carboxylic acid; |
| B-51 | 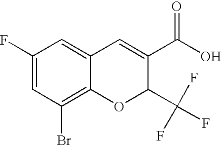 |
| | 8-bromo-6-fluoro-2-trifluoromethyl-2H-1-benzopyran-3- |
| | carboxylic acid; |
| B-52 | 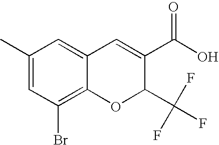 |
| | 8-bromo-6-methyl-2-trifluoromethyl-2H-1-benzopyran-3- |
| | carboxylic acid; |
| B-53 | 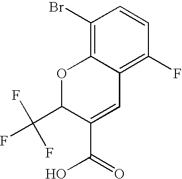 |
| | 8-bromo-5-fluoro-2-trifluoromethyl-2H-1-benzopyran-3- |
| | carboxylic acid; |
| B-54 | 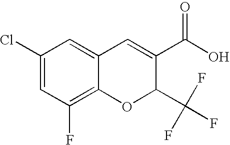 |
| | 6-chloro-8-fluoro-2-trifluoromethyl-2H-1-benzopyran-3- |
| | carboxylic acid; |
| B-55 | 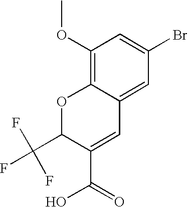 |
| | 6-bromo-8-methoxy-2-trifluoromethyl-2H-1-benzopyran-3- |
| | carboxylic acid; |
| B-56 | 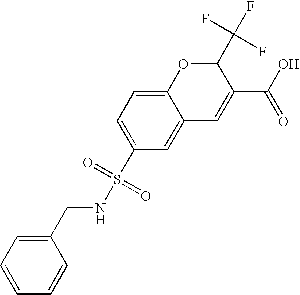 |
| | 6-[[(phenylmethyl)amino]sulfonyl]-2-trifluoromethyl-2H-1- |
| | benzopyran-3-carboxylic acid; |
| B-57 | 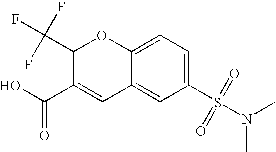 |
| | 6-[(dimethylamino)sulfonyl]-2-trifluoromethyl-2H-1- |
| | benzopyran-3-carboxylic acid; |
| B-58 | 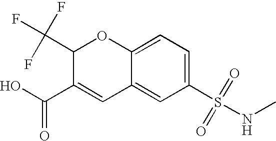 |
| | 6-[(methylamino)sulfonyl]-2-trifluoromethyl-2H-1-benzopyran- |
| | 3-carboxylic acid; |
| B-59 | 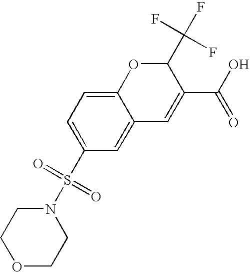 |
| | 6-[(4-morpholino)sulfonyl]-2-trifluoromethyl-2H-1-benzopyran- |
| | 3-carboxylic acid; |
| B-60 | 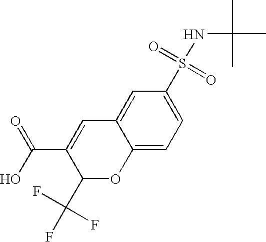 |
| | 6-[(1,1-dimethylethyl)aminosulfonyll-2-trifluoromethyl- |
| | 2H-1-benzopyran-3-carboxylic acid; |
| B-61 | 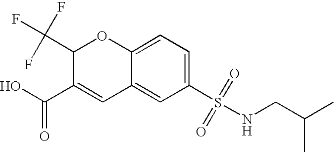 |
| | 6-[(2-methylpropyl)aminosulfonyl]-2-trifluoromethyl-2H-1- |
| | benzopyran-3-carboxylic acid, |
| B-62 | 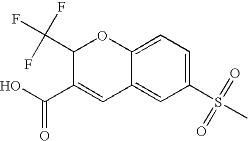 |
| | 6-methylsulfonyl-2-trifluoromethyl-2H-1-benzopyran-3- |
| | carboxylic acid; |
| B-63 | 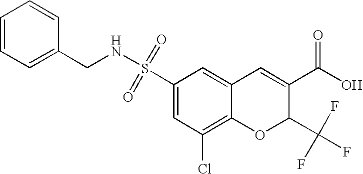 |
| | 8-chloro-6-[[(phenylmethyl)aminolsulfonyl]-2-trifluoromethyl- |
| | 2H-1-benzopyran-3-carboxylic acid; |
| B-64 | 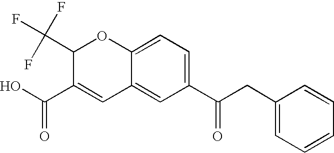 |
| | 6-phenylacetyl-2-trifluoromethyl-2H-1-benzopyran-3- |
| | carboxylic acid; |
| B-65 | 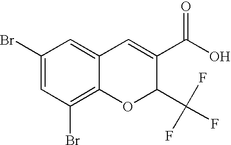 |
| | 6,8-dibromo-2-trifluoromethyl-2H-1-benzopyran-3- |
| | carboxhlic acid; |
| B-66 | 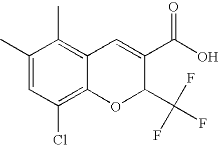 |
| | 8-chloro-5,6-dimethyl-2-trifluoromethyl-2H-1-benzopyran- |
| | 3-carboxylic acid; |
| B-67 | 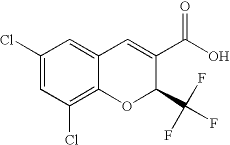 |
| | 6,8-dichloro-(S)-2-trifluoromethyl-2H-1-benzopyran-3- |
| | carboxylic acid; |
| B-68 | 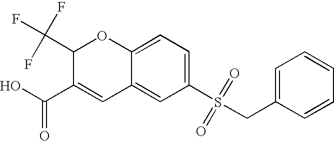 |
| | 6-benzylsulfonyl-2-trifluoromethyl-2H-1-benzopyran-3- |
| | carboxylic acid; |
| B-69 | 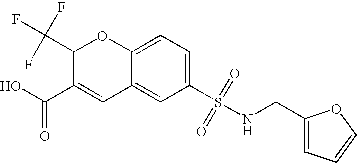 |
| | 6-[[N-(2-furylmethyl)amino]sulfonyl]-2-trifluoromethyl- |
| | 2H-1-benzopyran-3-carboxylic acid; |
| B-70 | 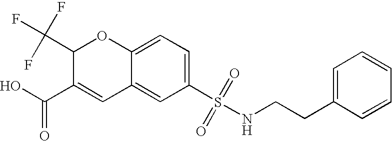 |
| | 6-[[N-(2-phenylethyl)amino]sulfonyl]-2-trifluoromethyl-2H- |
| | 1-benzopyran-3-carboxylic acid; |
| B-71 | 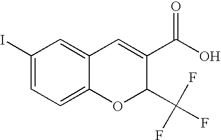 |
| | 6-iodo-2-trifluoromethyl-2H-1-benzopyran-3-carboxylic acid; |
| B-72 | 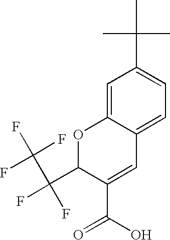 |
| | 7-(1,1-dimethylethyl)-2-pentafluoroethyl-2H-1-benzopyran- |
| | 3-carboxylic acid; |
| B-73 | 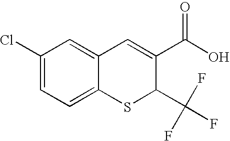 |
| | 6-chloro-2-trifluoromethyl-2H-1-benzothiopyran-3-carboxylic acid; |
| B-74 | 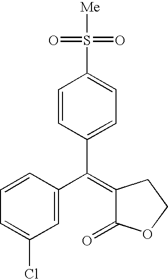 |
| | 3-[(3-chloro-phenyl)-(4-methanesulfonyl-phenyl)-methylene]- |
| | dihydro-furan-2-one or BMS-347070; |
| B-75 | 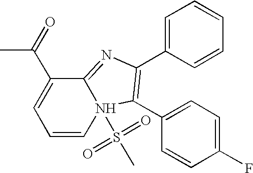 |
| | 8-acetyl-3-(4-fluorophenyl)-2-(4-methylsulfonyl)phenyl- |
| | imidazo(1,2-a)pyridine; |
| B-76 | 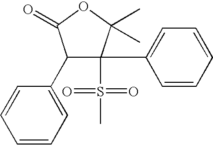 |
| | 5,5-dimethyl-4-(4-methylsulfonyl)phenyl-3-phenyl-2- |
| | (5H)-furanone; |
| B-77 | 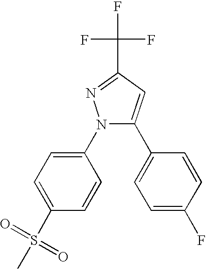 |
| | 5-(4-fluorophenyl)-1-[4-(methylsulfonyl)phenyl]-3- |
| | (trifluoromethyl)pyrazole; |
| B-78 | 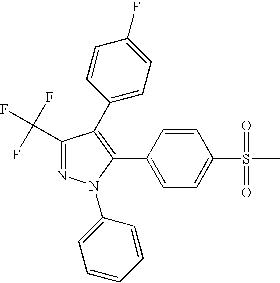 |
| | 4-(4-fluorophenyl)-5-[4-(methylsulfonyl)phenyl]-1- |
| | phenyl-3-(trifluoromethyl)pyrazole; |
| B-79 | 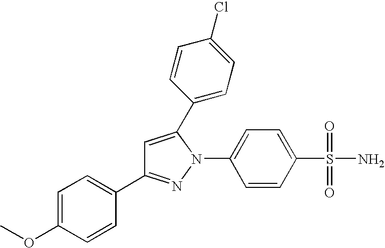 |
| | 4-(5-(4-chlorophenyl)-3-(4-methoxyphenyl)-1H-pyrazol-1-yl) |
| | benzenesulfonamide; |
| B-80 | 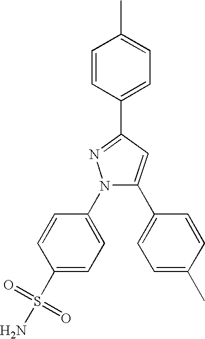 |
| | 4-(3,5-bis(4-methylphenyl)-1H-pyrazol-1-yl)benzenesulfonamide; |
| B-81 | 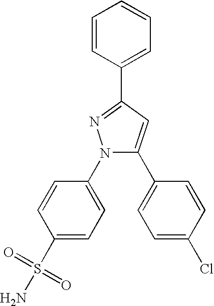 |
| | 4-(5-(4-chlorophenyl)-3-phenyl-1H-pyrazol-1-yl)benzenesulfonamide |
| B-82 | 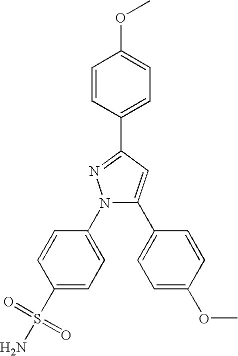 |
| | 4-(3,5-bis(4-methoxyphenyl)-1H-pyrazol-1-yl)benzenesulfonamide; |
| B-83 | 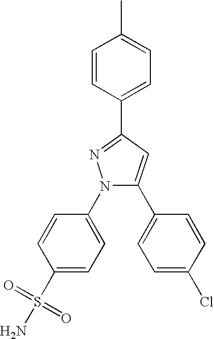 |
| | 4-(5-(4-chlorophenyl)-3-(4-methylphenyl)-1H-pyrazol-1- |
| | yl)benzenesulfonamide; |
| B-84 | 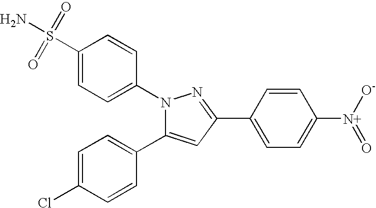 |
| | 4-(5-(4-chlorophenyl)-3-(4-nitrophenyl)-1H-pyrazol-1- |
| | yl)benzenesulfonamide; |
| B-85 | 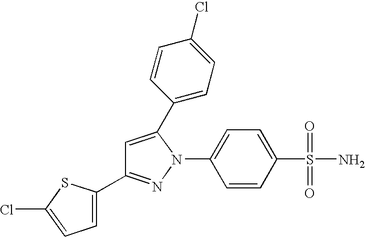 |
| | 4-(5-(4-chlorophenyl)-3-(5-chloro-2-thienyl)-1H-pyrazol- |
| | 1-yl)benzenesulfonamide; |
| B-86 | 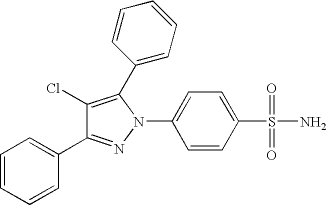 |
| | 4-(4-chloro-3,5-diphenyl-1H-pyrazol-1-yl)benzenesulfonamide; |
| B-87 | 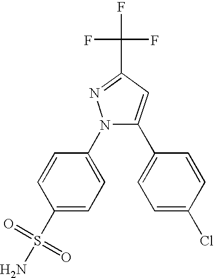 |
| | 4-[5-(4-chlorophenyl)-3-(trifluoromethyl)-1H-pyrazol-1- |
| | yl]benzenesulfonamide; |
| B-88 | 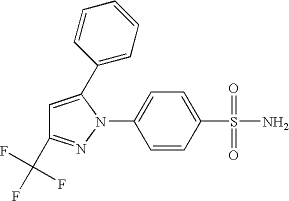 |
| | 4-[5-phenyl-3-(trifluoromethyl)-1H-pyrazol-1- |
| | yl]benzenesulfonamide; |
| B-89 | 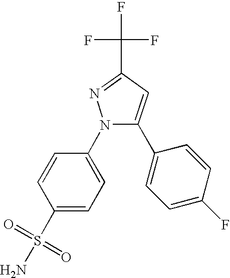 |
| | 4-[5-(4-fluorophenyl)-3-(trifluoromethyl)-1H- |
| | pyrazol-1-yl]benzenesulfonamide; |
| B-90 | 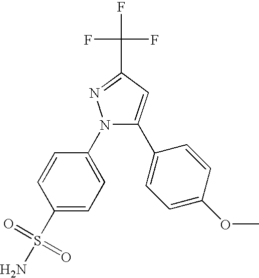 |
| | 4[5-(4-methoxyphenyl)-3-(trifluoromethyl)-1H-pyrazol-1- |
| | yl]benzenesulfonamide; |
| B-91 | 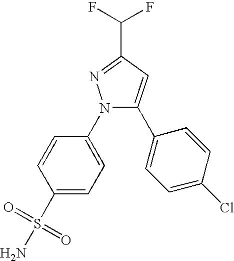 |
| | 4-[5-(4-chlorophenyl)-3-(difluoromethyl)-1H-pyrazol-1- |
| | yl]benzenesulfonamide; |
| B-92 | 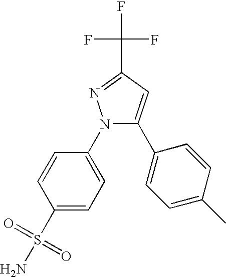 |
| | 4-[5-(4-methylphenyl)-3-(trifluoromethyl)-1H-pyrazol- |
| | 1-yl]benzenesulfonamide; |
| B-93 | 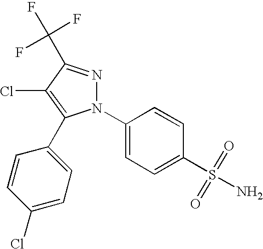 |
| | 4-[4-chloro-5-(4-chlorophenyl)-3-(trifluoromethyl)-1H- |
| | pyrazol-1-yl]benzenesulfonamide; |
| B-94 | 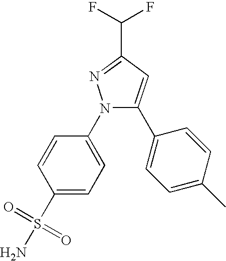 |
| | 4-[3-(difluoromethyl)-5-(4-methylphenyl)-1H-pyrazol-1- |
| | yl]benzenesulfonamide; |
| B-95 | 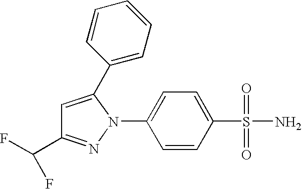 |
| | 4-[3-(difluoromethyl)-5-phenyl-1H-pyrazol-1- |
| | yl]benzenesulfonamide; |
| B-96 | 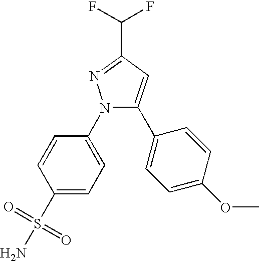 |
| | 4-[3-(difluoromethyl)-5-(4-methoxyphenyl)-1H-pyrazol-1- |
| | yl]benzenesulfonamide; |
| B-97 | 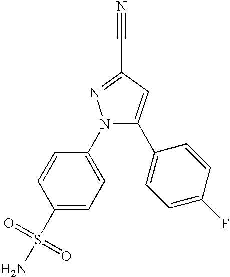 |
| | 4-[3-cyano-5-(4-fluorophenyl)-1H-pyrazol-1- |
| | yl]benzenesulfonamide; |
| B-98 | 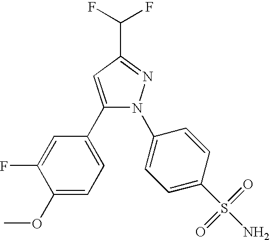 |
| | 4-[3-(difluoromethyl)-5-(3-fluoro-4-methoxyphenyl)-1H- |
| | pyrazol-1-yl]benzenesulfonamide; |
| B-99 | 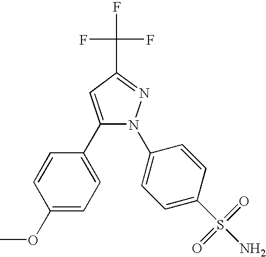 |
| | 4-[5-(3-fluoro-4-methoxyphenyl)-3-(trifluoromethyl)-1H- |
| | pyrazol-1-yl]benzenesulfonamide; |
| B-100 | 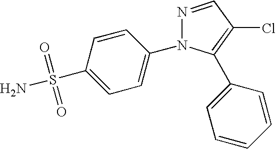 |
| | 4-[4-chloro-5-phenyl-1H-pyrazol-1-yl]benzenesulfonamide; |
| B-101 | 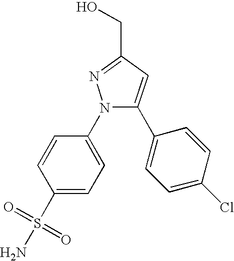 |
| | 4-[5-(4-chlorophenyl)-3-(hydroxymethyl)-1H-pyrazol-1- |
| | yl]benzenesulfonamide; |
| B-102 | 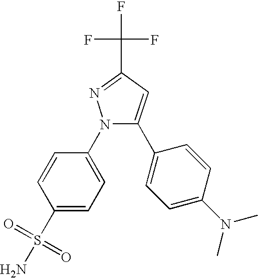 |
| | 4-[5-(4-(N,N-dimethylamino)phenyl)-3-(trifluoromethyl)- |
| | 1H-pyrazol-1-yl]benzenesulfonamide; |
| B-103 | 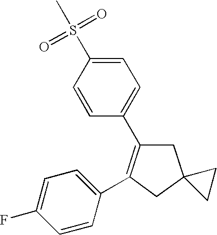 |
| | 5-(4-fluorophenyl)-6-[4-(methylsulfonyl)phenyl]spiro[2.4]hept-5-ene; |
| B-104 | 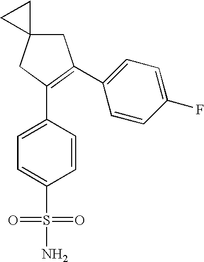 |
| | 4-[6-(4-fluorophenyl)spirof2.4]hept-5-en-5-yl]benzenesulfonamide; |
| B-105 | 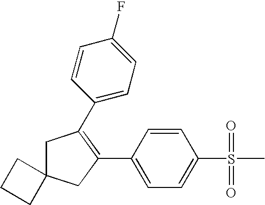 |
| | 6-(4-fluorophenyl)-7-[4-methylsulfonyl)phenyl]spiro[3.4]oct- |
| | 6-ene; |
| B-106 | 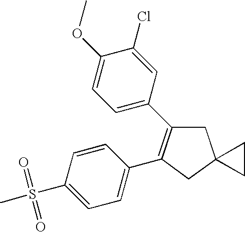 |
| | 5-(3-chloro-4-methoxyphenyl)-6-[4-(methylsulfonyl)phenyl] |
| | spiro[2.4]hept-5-ene; |
| B-107 | 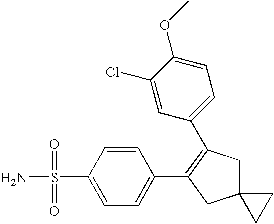 |
| | 4-[6-(3-chloro-4-methoxyphenyl)spiro[2.4]hept-5-en-5- |
| | yl]benzenesulfonamide; |
| B-108 | 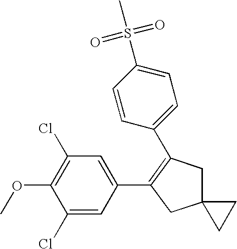 |
| | 5-(3,5-dichloro-4-methoxyphenyl)-6-[4-(methylsulfonyl)phenyl] |
| | spiro[2.4]hept-5-ene; |
| B-109 | 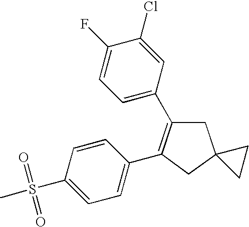 |
| | 5-(3-chloro-4-fluorophenyl)-6-[4-(methylsulfonyl)phenyl] |
| | spiro[2.4]hept-5-ene; |
| B-110 | 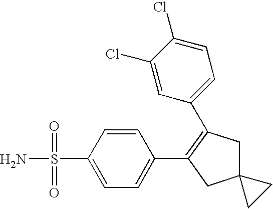 |
| | 4-[6-(3,4-dichlorophenyl)spiro[2.4]hept-5-en-5- |
| | yl]benzenesulfonamide |
| B-111 | 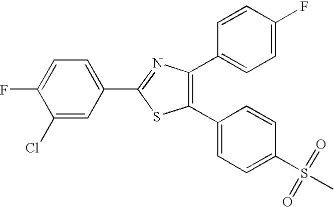 |
| | 2-(3-chloro-4-fluorophenyl)-4-(4-fluorophenyl-5-(4- |
| | methylsulfonylphenyl)thiazole; |
| B-112 | 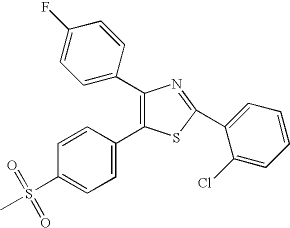 |
| | 2-(2-chlorophenyl)-4-(4-fluorophenyl)-5-(4- |
| | methylsulfonylphenyl)thiazole; |
| B-113 | 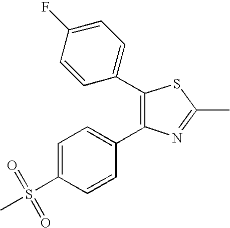 |
| | 5-(4-fluorophenyl)-4-(4-methylsulfonylphenyl)-2- |
| | methylthiazole; |
| B-114 | 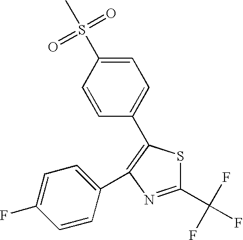 |
| | 4-(4-fluorophenyl)-5-(4-methylsulfonylphenyl)-2- |
| | trifluoromethylthiazole; |
| B-115 | 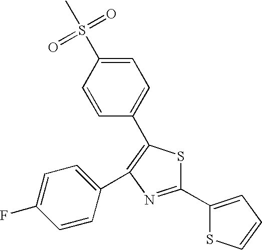 |
| | 4-(4-fluorophenyl)-5-(4-methylsulfonylphenyl)-2-(2- |
| | thienyl)thiazole; |
| B-116 | 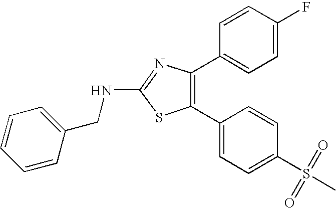 |
| | 4-(4-fluorophenyl)-5-(4-methylsulfonylphenyl)-2- |
| | benzylaminothiazole; |
| B-117 | 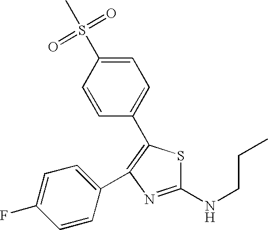 |
| | 4-(4-fluorophenyl)-5-(4-methylsulfonylphenyl)-2-(1- |
| | propylamino)thiazole; |
| B-118 | 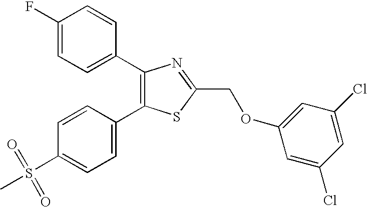 |
| | 2-((3,5-dichlorophenoxy)methyl)-4-(4-fluorophenyl)-5-[4- |
| | (methylsulfonyl)phenyl]thiazole; |
| B-119 | 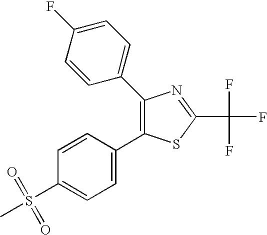 |
| | 5-(4-fluorophenyl)-4-(4-methylsulfonylphenyl)-2- |
| | trifluoromethylthiazole; |
| B-120 | 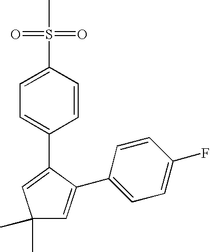 |
| | 1-methylsulfonyl-4-[1,1-dimethyl-4-(4-fluorophenyl) |
| | cyclopenta-2,4-dien-3-yl]benzene; |
| B-121 | 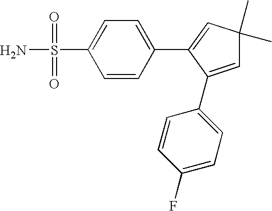 |
| | 4-[4-(4-fluorophenyl)-1,1-dimethylcyclopenta-2,4-dien- |
| | 3-yl]benzenesulfonamide; |
| B-122 | 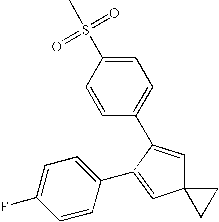 |
| | 5-(4-fluorophenyl)-6-[4-(methylsulfonyl)phenyl]spiro |
| | [2.4]hepta-4,6-diene; |
| B-123 | 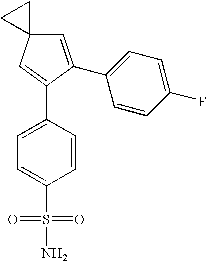 |
| | 4-[6-(4-fluorophenyl)spiro[2.4]hepta-4,6-dien-5- |
| | yl]benzenesulfonamide; |
| B-124 | 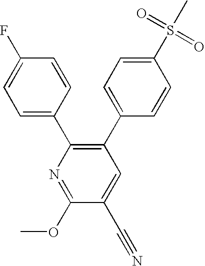 |
| | 6-(4-fluorophenyl)-2-methoxy-5-[4-(methylsulfonyl)phenyl]- |
| | pyridine-3-carbonitrile; |
| B-125 | 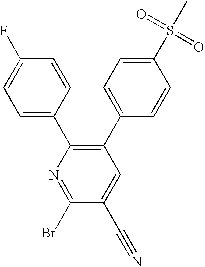 |
| | 2-bromo-6-(4-fluorophenyl)-5-[4-(methylsulfonyl)phenyl]- |
| | pyridine-3-carbonitrile; |
| B-126 | 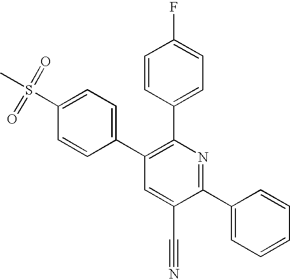 |
| | 6-(4-fluorophenyl)-5-[4-(methylsulfonyl)phenyl]-2-phenyl- |
| | pyridine-3-carbontrile; |
| B-127 | 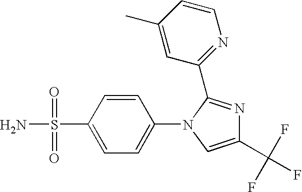 |
| | 4-[2-(4-methylpyridin-2-yl)-4-(trifluoromethyl)-1H- |
| | imidazol-1-yl]benzenesulfonamide; |
| B-128 | 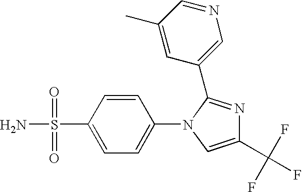 |
| | 4-[2-(5-methylpyridin-3-yl)-4-(trifluoromethyl)-1H- |
| | imidazol-1-yl]benzenesulfonamide; |
| B-129 | 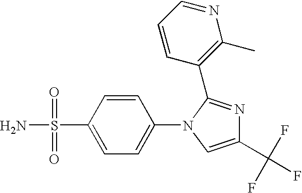 |
| | 4-[2-(2-methylpyridin-3-yl)-4-(trifluoromethyl)-1H- |
| | imidazol-1-yl]benzenesulfonamide; |
| B-130 | 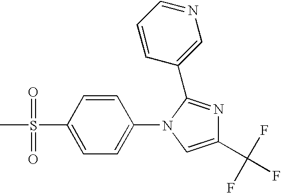 |
| | 3-[1-[4-(methylsulfonyl)phenyl]-4-(trifIuoromethyl)-1H- |
| | imidazol-2-yl]pyridine; |
| B-131 | 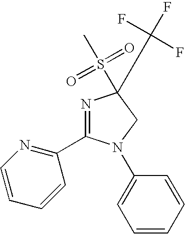 |
| | 2-[1-[4-(methylsulfonyl)phenyl-4-(trifluoromethyl)]-1H- |
| | imidazol-2-yl]pyridine; |
| B-132 | 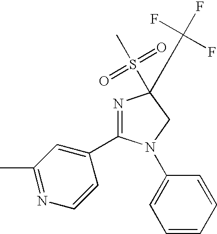 |
| | 2-methyl-4-[1-[4-(methylsulfonyl)phenyl-4-(trifluoromethyl)]- |
| | 1H-imidazol-2-yl]pyridine; |
| B-133 | 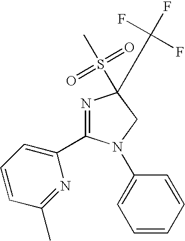 |
| | 2-methyl-6-[1-[4-(methylsulfonyl)phenyl-4-(trifluoromethyl)]- |
| | 1H-imidazol-2-yl]pyridine; |
| B-134 | 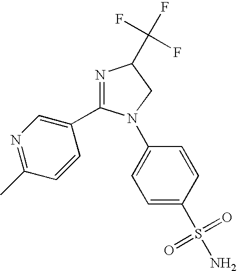 |
| | 4-[2-(6-methylpyridin-3-yl)-4-(trifluoromethy1)-1H-imidazol- |
| | 1-yl]benzenesulfonamide; |
| B-135 | 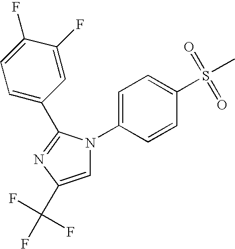 |
| | 2-(3,4-difluorophenyl)-1-[4-(methylsulfonyl)phenyl]- |
| | 4-(trifluoromethyl)-1H-imidazole; |
| B-136 | 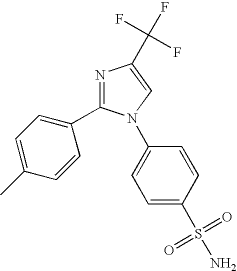 |
| | 4-[2-(4-methylphenyl)-4-(trifluoromethyl)-1H-imidazol-1- |
| | yl]benzenesulfonamide; |
| B-137 | 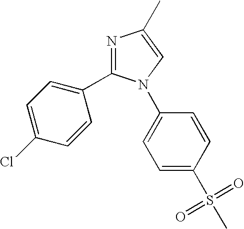 |
| | 2-(4-chlorophenyl)-1-[4-(methylsulfonyl)phenyl]-4-methyl- |
| | 1H-imidazole; |
| B-138 | 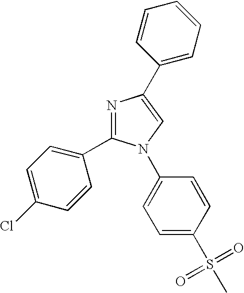 |
| | 2-(4-chlorophenyl)-1-[4-(methylsulfonyl)phenyl]-4- |
| | phenyl-1H-imidazole; |
| B-139 | 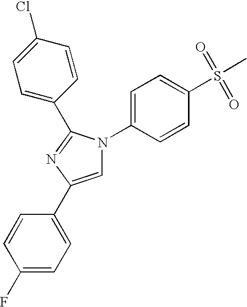 |
| | 2-(4-chlorophenyl)-4-(4-fluorophenyl)-1-[4-(methylsulfonyl) |
| | phenyl]-1H-imidazole; |
| B-140 | 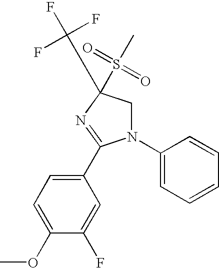 |
| | 2-(3-fluoro-4-methoxyphenyl)-1-[4-(methylsulfonyl)phenyl- |
| | 4-(trifluoromethyl)]-1H-imidazole; |
| B-141 | 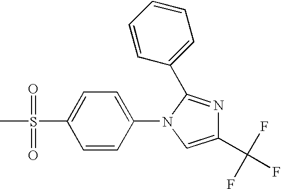 |
| | 1-[4-(methylsulfonyl)phenyl]-2-phenyl-4-trifluoromethyl- |
| | 1H-imidazole; |
| B-142 | 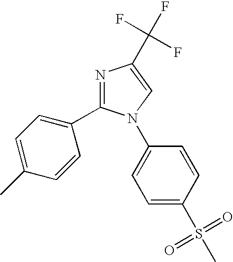 |
| | 2-(4-methylphenyl)-1-[4-(methylsulfonyl)phenyl]-4-trifluoromethyl- |
| | 1H-imidazole; |
| B-143 | 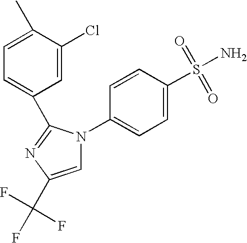 |
| | 4-[2-(3-chloro-4-methylphenyl)-4-(trifluoromethyl)- |
| | 1H-imidazol-1-yl]benzenesulfonamide; |
| B-144 | 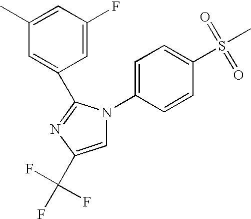 |
| | 2-(3-fluoro-5-methylphenyl)-1-[4-(methylsulfonyl)phenyl]- |
| | 4-(trifluoromethyl)-1H-imidazole; |
| B-145 | 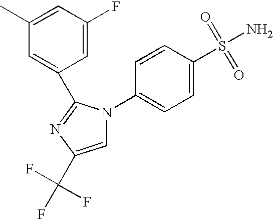 |
| | 4-[2-(3-fluoro-5-methylphenyl)-4-(trifluoromethyl- |
| | 1H-imidazole-1-yl]benzenesulfonamide; |
| B-146 | 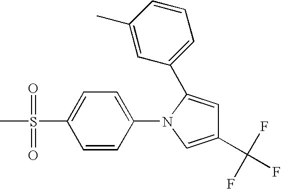 |
| | 2-(3-methylphenyl)-1-[4-(methylsulfonyl)phenyl]-4- |
| | trifluoromethyl-1H-imidazole; |
| B-147 | 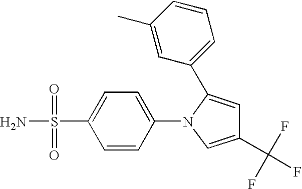 |
| | 4-[2-(3-methylphenyl)-4-trifluoromethyl-1H-imidazol-1- |
| | yl]benzenesulfonamide; |
| B-148 | 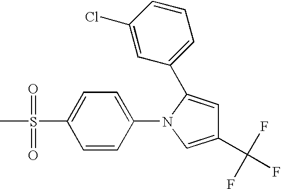 |
| | 1-[4-(methylsulfonyl)phenyl]-2-(3-chlorophenyl)-4- |
| | trifluoromethyl-1H-imidazole |
| B-149 | 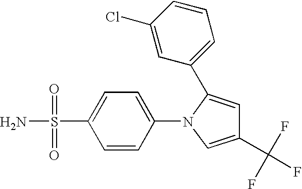 |
| | 4-[2-(3-chlorophenyl)-4-trifluoromethyl-1H-imidazol-1- |
| | yl]benzenesulfonamide; |
| B-150 | 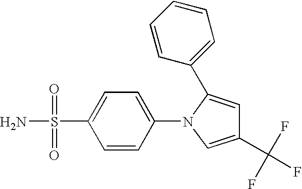 |
| | 4-[2-phenyl-4-trifluoromethyl-1H-imidazol-1- |
| | yl]benzenesulfonamide; |
| B-151 | 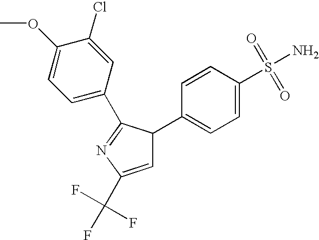 |
| | 4-[2-(4-methoxy-3-chlorophenyl)-4-trifluoromethyl-1H- |
| | imidazol-1-yl]benzenesulfonamide; |
| B-152 | 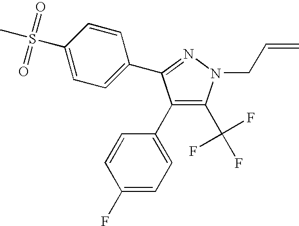 |
| | 1-allyl-4-(4-fluorophenyl)-3-[4-(methylsulfonyl)Phenyl]- |
| | 5-(trifluoromethyl)-1H-pyrazole; |
| B-153 | 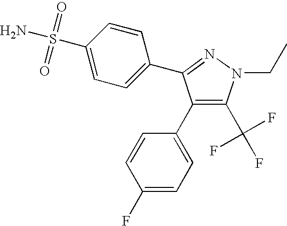 |
| | 4-[1-ethyl-4-(4-fluorophenyl)-5-(trifluoromethyl)-1H- |
| | pyrazol-3-yl]benzenesulfonamide; |
| B-154 | 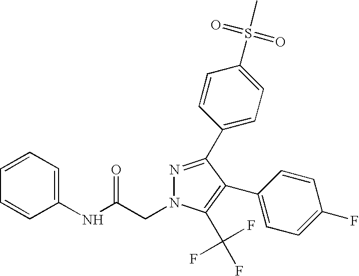 |
| | N-phenyl-[4-(4-fluorophenyl)-3-[4-(methylsulfonyl)phenyl]- |
| | 5-(trifluoromethyl)-1H-pyrazol-1-yl]acetamide; |
| B-155 | 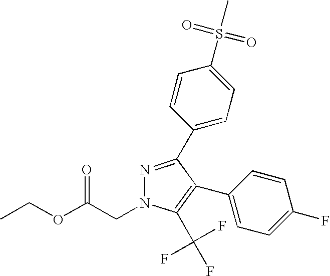 |
| | ethyl[4-(4-fluorophenyl)-3-[4-(methylsulfonyl)phenyl]- |
| | 5-(trifluoromethyl)-1H-pyrazol-1-yl]acetate; |
| B-156 | 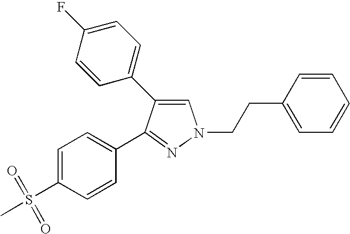 |
| | 4-(4-fluorophenyl)-3-[4-(methylsulfonyl)pheny1]-1-(2- |
| | phenylethyl)-1H-pyrazole; |
| B-157 | 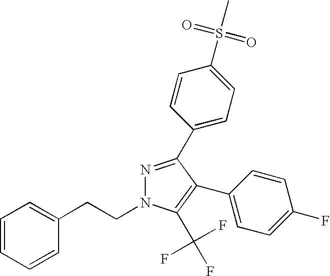 |
| | 4-(4-fluorophenyl)-3-[4-(methylsulfonyl)phenyl]- |
| | 1-(2-phenylethyl)-5-(trifluoromethyl)pyrazole; |
| B-158 | 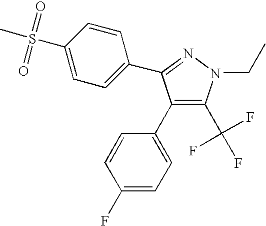 |
| | 1-ethyl-4-(4-fluorophenyl)-3-[4-methylsulfonyl)phenyl]- |
| | 5-(trifluoromethyl)-1H-pyrazole; |
| B-159 | 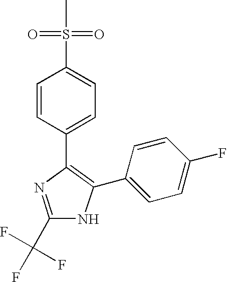 |
| | 5-(4-fluorophenyl)-4-(4-methylsulfonylphenyl)- |
| | 2-trifluoromethyl-1H-imidazole; |
| B-160 | 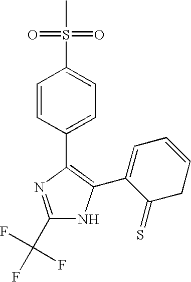 |
| | 4-[4-(methylsulfonyl)phenyl]-5-(2-thiophenyl)-2- |
| | (trifluoromethyl)-1H-imidazole; |
| B-161 | 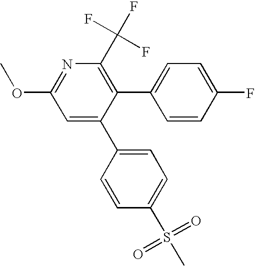 |
| | 5-(4-fluorophenyl)-2-methoxy-4-[4-(methylsulfonyl)phenyl]- |
| | 6-(trifluoromethyl)pyridine; |
| B-162 | 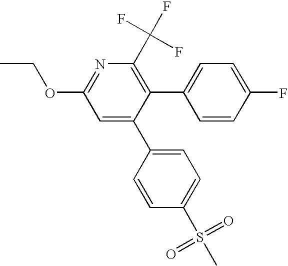 |
| | 2-ethoxy-5-(4-fluorophenyl)-4-[4-(methyl sulfonyl)phenyl]- |
| | 6-(trifluoromethyl)pyridine; |
| B-163 | 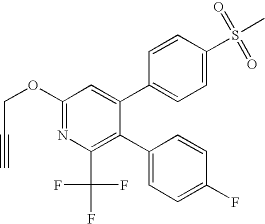 |
| | 5-(4-fluorophenyl)-4-[4-(methylsulfonyl)phenyl]- |
| | -(2-propynyloxy)-6-(trifluoromethyl)pyridine; |
| B-164 | 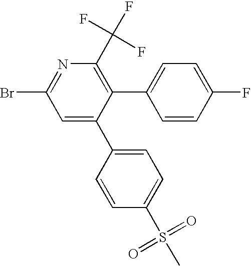 |
| | 2-bromo-5-(4-fluorophenyl)-4-[4-(methylsulfonyl)phenyl]- |
| | 6-(trifluoromethyl)pyridine; |
| B-165 | 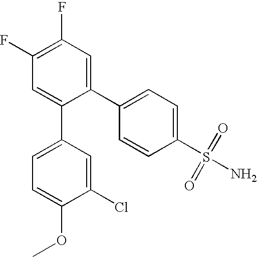 |
| | 4-[2-(3-chloro-4-methoxyphenyl-4,5-difluorophenyl] |
| | benzenesulfonamide; |
| B-166 | 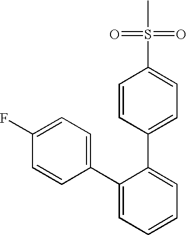 |
| | 1-(4-fluorophenyl)-2-[4-methylsulfonyl)phenyl]benzene; |
| B-167 | 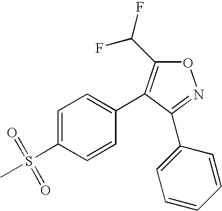 |
| | 5-difluoromethyl-4-(4-methylsulfonylphenyl)-3-phenylisoxazole; |
| B-168 | 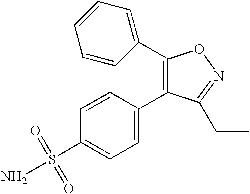 |
| | 4-[3-ethyl-5-phenylisoxazol-4-yl]benzenesulfonamide; |
| B-169 | 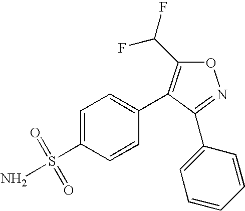 |
| | 4-[5-difluoromethyl-3-phenylisoxazol-4-yl]benzenesulfonamide; |
| B-170 | 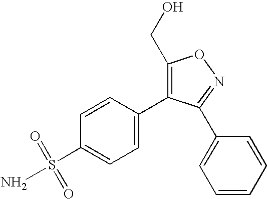 |
| | 4-[5-hydroxymethyl-3-phenylisoxazol-4-yl]benzenesulfonamide; |
| B-171 | 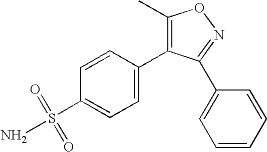 |
| | 4-[5-methyl-3-phenyl-isoxazo1-4-yl]benzenesulfonamide; |
| B-172 | 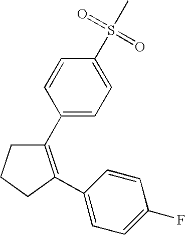 |
| | 1-[2-(4-fluorophenyl)cyclopenten-1-yl]-4-(methylsulfonyl)benzene; |
| B-173 | 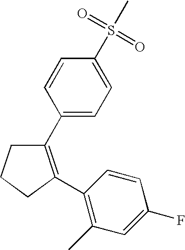 |
| | 1-[2-(4-fluoro-2-methylpheny])cyclopenten-1-yl]-4- |
| | (methylsulfonyl)benzene; |
| B-174 | 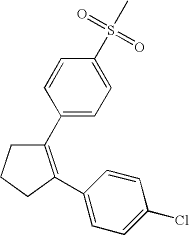 |
| | 1-[2-(4-chlorophenyl)cyclopenten-1-yl]-4-(methylsulfonyl)benzene; |
| B-175 | 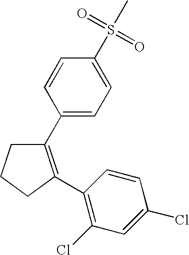 |
| | 1-[2-(2,4-dichlorophenyl)cyclopenten-1-yl]-4- |
| | (methylsulfonyl)benzene; |
| B-176 | 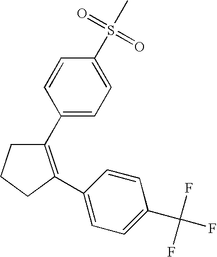 |
| | 1-[2-(4-trifloromethylphenyl)cyclopenten-1-yl]-4- |
| | (methylsulfonyl)benzene; |
| B-177 | 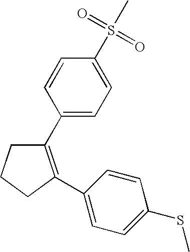 |
| | 1-[2-(4-methylthiophenyl)cyclopenten-1-yl]-4- |
| | (methylsulfonyl)benzene; |
| B-178 | 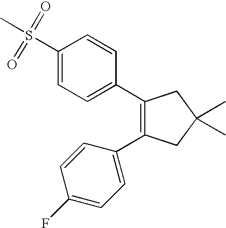 |
| | 1-[2-(4-fluorophenyl)-4,4-dimethylcyclopenten-1-yl]- |
| | 4-(methylsulfonyl)benzene; |
| B-179 | 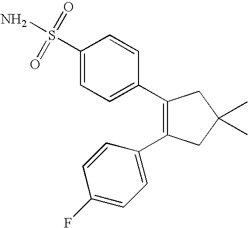 |
| | 4-[2-(4-fluorophenyl)-4,4-dimethylcyclopenten-1- |
| | yl]benzenesulfonamide; |
| B-180 | 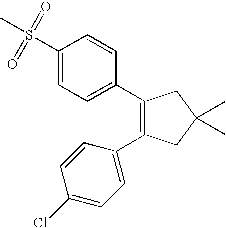 |
| | 1-[2-(3-chlorophenyl)-4,4-dimethylcyclopenten-1- |
| | yl]-4-(methylsulfonyl)benzene; |
| B-181 | 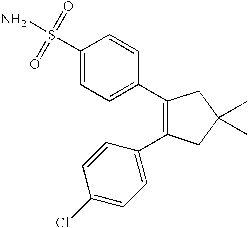 |
| | 4-[2-(4-chlorophenyl)-4,4-dimethylcyclopenten-1- |
| | yl]benzenesulfonamide; |
| B-182 | 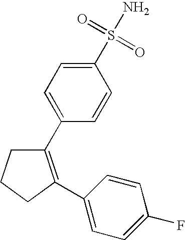 |
| | 4-[2-(4-fluorophenyl)cyclopenten-1- |
| | yl]benzenesulfonamide; |
| B-183 | 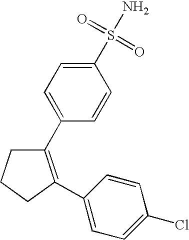 |
| | 4-[2-(4-chlorophenyl)cyclopenten-1- |
| | yl]benzenesulfonamide; |
| B-184 | 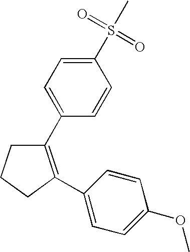 |
| | 1-[2-(4-methoxyphenyl)cyclopenten-1-yl]-4- |
| | (methylsulfonyl)benzene; |
| B-185 | 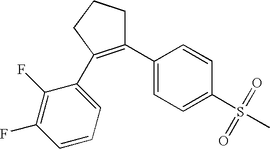 |
| | 1-[2-(2,3-difluorophenyl)cyclopenten-1-yl]-4- |
| | (methylsulfonyl)benzene; |
| B-186 | 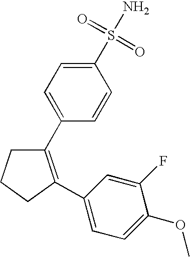 |
| | 4-[2-(3-fluoro-4-methoxyphenyl)cyclopenten-1-yl] |
| | benzenesulfonamide; |
| B-187 | 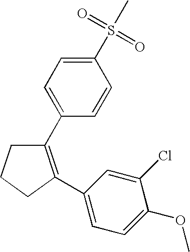 |
| | 1-[2-(3-chloro-4-methoxyphenyl)cyclopenten-1-yl]-4- |
| | (methylsulfonyl)benzene; |
| B-188 | 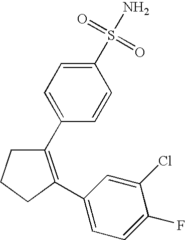 |
| | 4-[2-(3-chloro-4-fluorophenyl)cyclopenten-1- |
| | yl]benzenesulfonamide; |
| B-189 | 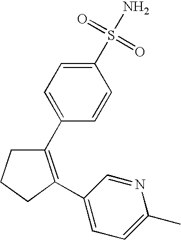 |
| | 4-[2-(2-methylpyridin-5-yl)cyclopenten-1- |
| | yl]benzenesulfonamide; |
| B-190 | 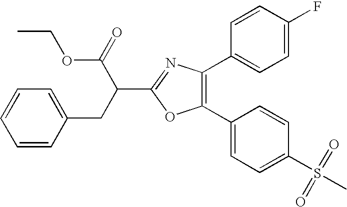 |
| | ethyl 2-[4-(4-fluorophenyl)-5-[4-(methylsulfonyl)phenyl]oxazol-2- |
| | yl]-2-benzyl-acetate; |
| B-191 | 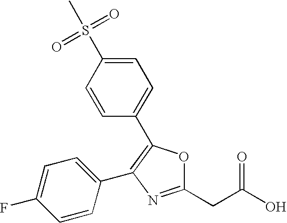 |
| | 2-[4-(4-fluorophenyl)-5-[4-(methylsulfonyl)phenyl]oxazol- |
| | 2-yl]acetic acid; |
| B-192 | 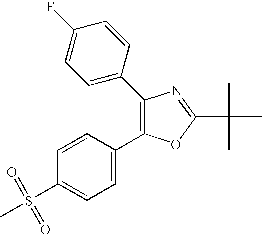 |
| | 2-(tert-butyl)-4-(4-fluorophenyl)-5-[4-(methylsulfonyl) |
| | phenyl]oxazole; |
| B-193 | 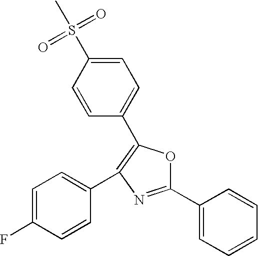 |
| | 4-(4-fluorophenyl)-5-[4-(methylsulfonyl)phenyl]-2-phenyloxzole; |
| B-194 | 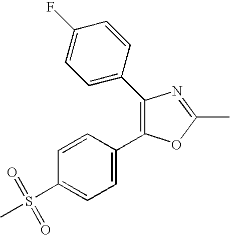 |
| | 4-(4-fluorophenyl)-2-methyl-5-[4-(methylsulfonyl) |
| | phenyl]oxazole; |
| B-195 | 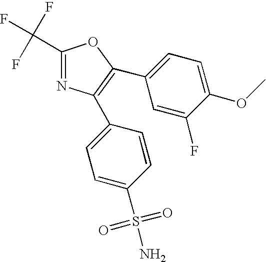 |
| | 4-[5-(3-fluoro-4-methoxyphenyl)-2-trifluoromethyl-4- |
| | oxazolyl]benzenesulfonamide; |
| B-196 | 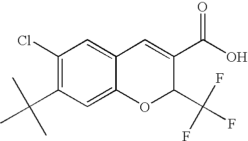 |
| | 6-chloro-7-(1,1-dimethylethyl)-2-trifluoromethyl-2H- |
| | 1-benzopyran-3-carboxylic acid; |
| B-197 | 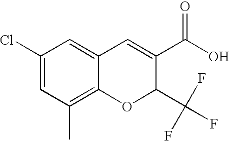 |
| | 6-chloro-8-methyl-2-trifluoromethyl-2H-1-benzopyran- |
| | 3-carboxylic acid; |
| B-198 | 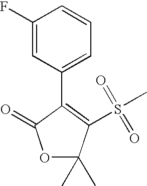 |
| | 5,5-dimethyl-3-(3-fluorophenyl)-4-methylsulfonyl-2(5H)- |
| | furanone; |
| B-199 | 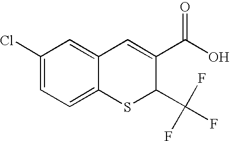 |
| | 6-chloro-2-trifluoromethyl-2H-1-benzothiopyran-3- |
| | carboxylic acid; |
| B-200 | 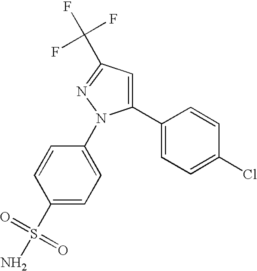 |
| | 4-[5-(4-chlorophenyl)-3-(trifluoromethyl)-1H-pyrazol- |
| | 1-yl]benzenesulfonamide; |
| B-201 | 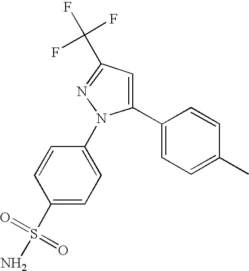 |
| | 4-[5-(4-methylphenyl)-3-(trifluoromethyl)-1H-pyrazol-1- |
| | yl]benzenesulfonamide; |
| B-202 | 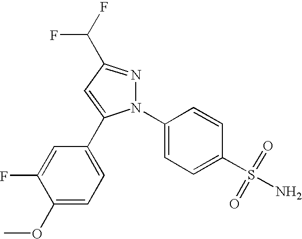 |
| | 4-[5-(3-fluoro-4-methoxyphenyl)-3-(difluoromethyl)- |
| | 1H-pyrazol-1-yl]benzenesulfonamide; |
| B-203 | 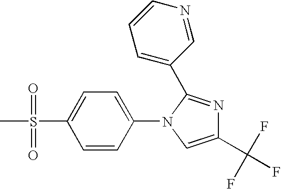 |
| | 3-[1-[4-methylsulfonyl)phenyl]-4-trifluoromethyl-1H- |
| | imidazol-2-yl]pyridine; |
| B-204 | 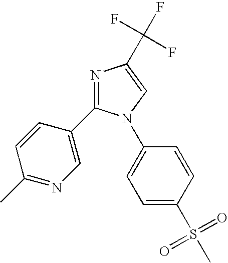 |
| | 2-methyl-5-[1-[4-(methylsulfonyl)phenyl]-4-trifluoromethyl- |
| | 1H-imidazol-2-yl]pyridine; |
| B-205 | 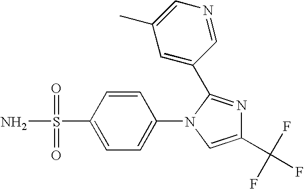 |
| | 4-[2-(5-methylpyridin-3-yl)-4-(trifluoromethyl)- |
| | 1H-imidazol-1-yl]benzenesulfonamide; |
| B-206 | 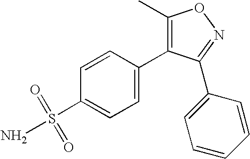 |
| | 4-[5-methyl-3-phenylisoxazol-4-yl]benzenesulfonamide; |
| B-207 | 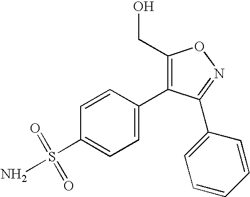 |
| | 4-[5-hydroxymethyl-3-phenylisoxazol-4-yl]benzenesulfonamide; |
| B-208 | 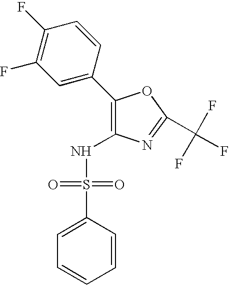 |
| | [2-trifluoromethyl-5-(3,4-difluorophenyl)-4- |
| | oxazolyl]benzenesulfonamide; |
| B-209 | 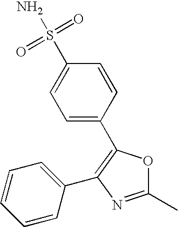 |
| | 4-[2-methyl-4-phenyl-5-oxazolyl]benzenesulfonamide; |
| B-210 | 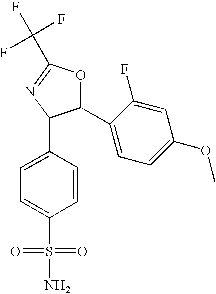 |
| | 4-[5-(2-fluoro-4-methoxyphenyl)-2-trifluoromethyl-4- |
| | oxazolyl]benzenesulfonamide; |
| B-211 | 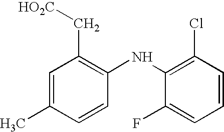 |
| B-212 | 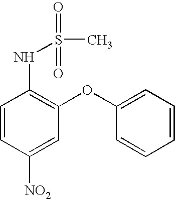 |
| | N-(4-nitro-2-phenoxy-phenyl)-methanesulfonamide or |
| | Nimesulide |
| B-213 | 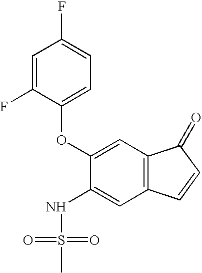 |
| | N-[6-(2,4-difluoro-phenoxy)-1-oxo-inden-5-yl]- |
| | methanesulfonamide or Flosulide |
| B-214 | 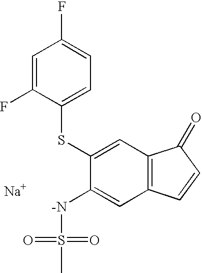 |
| | N-[6-(2,4-difluoro-phenylsulfanyl)-1-oxo-1H-inden-5- |
| | yl]-methanesulfonamide, soldium salt, or L-745337 |
| B-215 | 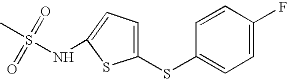 |
| | N-[5-(4-fluoro-phenylsulfanyl)-thiophen-2- |
| | yl]-methanesulfonamide or RWJ-63556 |
| B-216 | 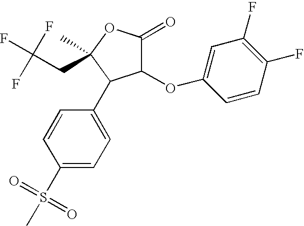 |
| | 3-(3,4-difluoro-phenoxy)-4-(4-methanesulfonyl-phenyl)-5- |
| | methyl-5-(2,2,2-trifluoro-ethyl)-5H-furan-2-one or |
| | L-784512 |
| B-217 | 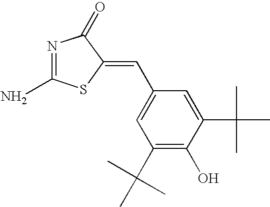 |
| | (5Z)-2-amino-5-[[3,5-bis(1,1-dimethylethyl)-4- |
| | hydroxypbenyl]methylene]-4(5H)-thiazolone or |
| | Darbufelone |
| B-218 | CS-502 |
| B-219 | LAS-34475 |
| B-220 | LAS-34555 |
| B-221 | S-33516 |
| B-222 | SD-8381 |
| B-223 | L-783003 |
| B-224 | 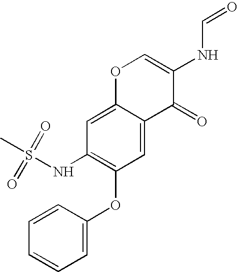 |
| | N-[3-(formylamino)-4-oxo-6-phenoxy-4H-1-benzopyran-7- |
| | yl]-methanesulfonamide or T614 |
| B-225 | D-1367 |
| B-226 | L-748731 |
| B-227 | 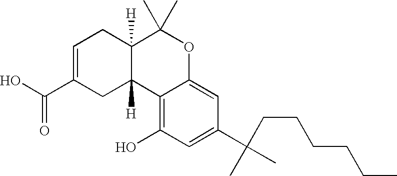 |
| | (6aR,10aR)-3-(1,1-dimethylheptyl)-6a,7,10,10a-tetrahydro- |
| | 1-hydroxy-6,6-dimethyl-6H-dibenzo[b,d]pyran-9- |
| | carboxylic acid or CT3 |
| B-228 | CGP-28238 |
| B-229 | 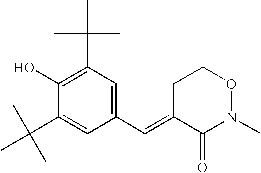 |
| | 4-[[3,5-bis(1,1-dimethylethyl)-4-hydroxyphenyl]methylene] |
| | dihydro-2-methyl-2H-1,2-oxazin-3(4H)-one or BF-389 |
| B-230 | GR-253035 |
| B-231 | 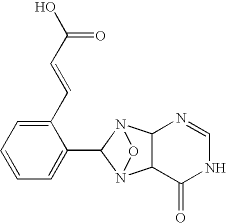 |
| | 2-(6-dioxo-9H-purin-8-yl)cinnamic acid |
| B-232 | S-2474 |
| B-233 | 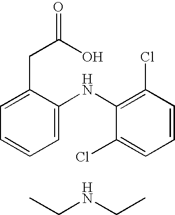 |

The cyclooxygenase-2 selective inhibitor employed in the present invention can exist in tautomeric, geometric or stereoisomeric forms. Generally speaking, suitable cyclooxygenase-2 selective inhibitors that are in tautomeric, geometric or stereoisomeric forms are those compounds that inhibit cyclooxygenase-2 activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, l-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

The cyclooxygenase-2 selective inhibitors utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

The cyclooxygenase-2 selective inhibitors useful in the practice of the present invention can be formulated into pharmaceutical compositions and administered by any means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrastemal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

Solid dosage forms for oral administration may include capsules, tablets, pills, powders, and granules. In such solid dosage forms, the compounds are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the cyclooxygenase-2 selective inhibitor will vary depending upon the patient and the particular mode of administration. In general, the pharmaceutical compositions may contain a cyclooxygenase-2 selective inhibitor in the range of about 0.1 to 2000 mg, more typically, in the range of about 0.5 to 500 mg and still more typically, between about 1 and 200 mg. A daily dose of about 0.01 to 100 mg/kg body weight, or more typically, between about 0.1 and about 50 mg/kg body weight and even more typically, from about 1 to 20 mg/kg body weight, may be appropriate. The daily dose can be administered in one to about four doses per day.

In one embodiment, when the cyclooxygenase-2 selective inhibitor comprises rofecoxib, it is typical that the amount used is within a range of from about 0.15 to about 1.0 mg/day·kg, and even more typically, from about 0.18 to about 0.4 mg/day·kg.

In still another embodiment, when the cyclooxygenase-2 selective inhibitor comprises etoricoxib, it is typical that the amount used is within a range of from about 0.5 to about 5 mg/day·kg, and even more typically, from about 0.8 to about 4 mg/day·kg.

Further, when the cyclooxygenase-2 selective inhibitor comprises celecoxib, it is typical that the amount used is within a range of from about 1 to about 20 mg/day·kg, even more typically, from about 1.4 to about 8.6 mg/day·kg, and yet more typically, from about 2 to about 3 mg/day·kg.

When the cyclooxygenase-2 selective inhibitor comprises valdecoxib, it is typical that the amount used is within a range of from about 0. 1 to about 5 mg/day·kg, and even more typically, from about 0.8 to about 4 mg/day·kg.

In a further embodiment, when the cyclooxygenase-2 selective inhibitor comprises parecoxib, it is typical that the amount used is within a range of from about 0.1 to about 5 mg/day·kg, and even more typically, from about 1 to about 3 mg/day·kg.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

Carbonic Anhydrase Inhibitors

A number of suitable carbonic anhydrase inhibitors or pharmaceutically acceptable salts or prodrugs thereof may be employed in the method of the present invention. Typically, the carbonic anhydrase inhibitor employed does not inhibit cyclooxygenase-2. In one embodiment, the carbonic anhydrase inhibitor can be, for example, methazolamide, Formula A-1 (CAS registry number 554-57-4) or a pharmaceutically acceptable salt or prodrug thereof.

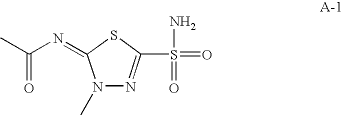

In another embodiment, the carbonic anhydrase inhibitor can be, for example, acetazolamide, Formula A-2 (CAS registry number 59-66-5) or pharmaceutically acceptable salt or prodrug thereof.

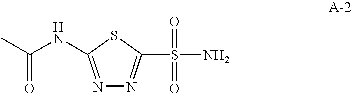

In yet another embodiment, the carbonic anhydrase inhibitor can be, for example, dichlorphenamide Formula A-3 (CAS registry number 120-97-8) or a pharmaceutically acceptable salt or prodrug thereof.

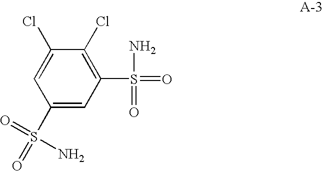

In a further embodiment the carbonic anhydrase inhibitor is selected from the group consisting of benzothiazole sulfonamides having the general Formula I shown below and possessing, by way of example and not limitation, the structures disclosed in Table 1. Furthermore, benzothiazole sulfonamide carbonic anhydrase inhibitors useful in the practice of the present methods are described in U.S. Pat. Nos. 4,975,449 and 5,059,613, both of which are herein incorporated by reference in their entirety.

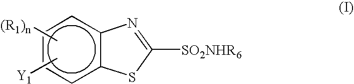

wherein:

each R₁ is hydrogen, lower alkyl, halogen, nitro, trihaloalkyl, lower alkoxy, formyl, lower alkanoyl loweralkylamino or diloweralkylamino;

R₆ is hydrogen or lower alkyl;

Y₁ is:

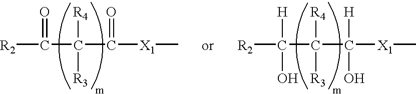

wherein:

X₁ is O or NR₅ or S;

R₂ is OR₇ or NR₇ R₈;

each R₃ and R₄ are hydrogen or lower alkyl;

R₅, R₇ and R₈ are independently hydrogen or lower alkyl;

m is an integer which is 0, 1, 2, 3, 4, 5, or 6, and

n is an integer which is 0, 1, 2, or 3.

TABLE 1

| Com- | |
|---|---|
| pound | |
| No. | Compound |
| A-4 | 6-hydroxy-2-benzothiazole sulfonamide |
| A-5 | 6-(ethyloxalyloxy)-2-benzothiazole sulfonamide |
| A-6 | 6-(ethylsuccinyloxy)-2-benzothiazole sulfonamide |
| A-7 | 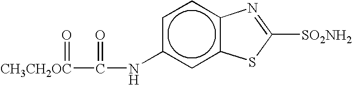 |
| A-8 | 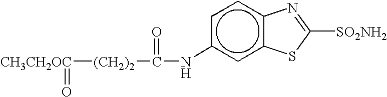 |

In another embodiment, the carbonic anhydrase inhibitor is selected from the class of benzothiazolesulfonamide carbonic anhydrase inhibitors represented by the general structure of Formula IIa shown below and possessing, by way of example and not limitation, the structures disclosed in Table 2a. Furthermore, benzothiazolesulfonamide carbonic anhydrase inhibitors useful in the practice of the present methods are described in U.S. Pat. Nos. 5,095,026 and 5,157,044, both of which are herein incorporated by reference in their entirety.

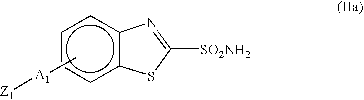

wherein:

Z₁ represents a water soluble carrier, and

A₁ is a moiety which is attached to the carbonic anhydrase inhibitor which allows it to still retain carbonic anhydrase inhibitory activity, but also form an enzymatically cleavable bond between A₁ and Z₁.

TABLE 2A

| Compound No. | Compound |
|---|---|
| A-9 | N-methyl-2-benzothiazolesulfonamide |
| A-10 | N-acetyl-2-benzothiazole-sulfonamide |
| A-11 | N-acetyl-6-ethoxy-2-benzothiazolesulfonamide |
| A-12 | 6-ethoxy-N-methyl-2-benzothiazolesulfonamide |
| A-13 | 6-ethoxy-N-propyl-2-benzothiazolesulfonamide |
| A-14 | 6-hydroxy-N-methyl-2-benzothiazolesulfonamide |
| A-15 | 6-hydroxy-2-benzothiazole-sulfonamide |
| A-16 | 6-chloro-2-benzothiazolesulfonamide |
| A-17 | 6-0-acetyl-2-acetyl-2-benzothiazolesulfonamide |
| A-18 | 6-hydroxyethoxybenzothiazolesulfonamide |
| A-19 | 6-benzyloxybenzothiazole-2-sulfonamide |
| A-20 | 7-chloro-2-benzothiazolesulfonamide |
| A-21 | 6 amino-benzothiazolesulfonamide |
| A-22 | 6-fluoro-2-benzothiazolesulfonamide |
| A-23 | 6-bromo-2-benzothiazolesulfonamide |
| A-24 | 4,6-dichloro-2-benzothiazolesulfonamide |

In yet another embodiment, the carbonic anhydrase inhibitor is selected from the class of hydroxymethazolamide carbonic anhydrase inhibitors represented by the general structure of Formula IIb shown below and possessing, by way of example and not limitation, the structures disclosed in Table 2b. Furthermore, hydroxymethazolamide carbonic anhydrase inhibitors useful in the practice of the present methods are described in U.S. Pat. Nos. 5,095,026 and 5,157,044, both of which are herein incorporated by reference in their entirety.

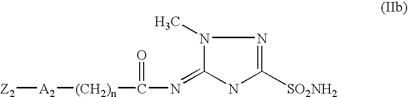

wherein:

Z₂ represents a water soluble carrier,

N is 1,2,3,4, or 5; and

A₂ is a moiety which is attached to the carbonic anhydrase inhibitor which allows it to still retain carbonic anhydrase inhibitory activity, but also form an enzymatically cleavable bond between A₂ and Z₂.

TABLE 2B

| Compound No. | Compound |
|---|---|
| A-25 | Hydroxymethazolamide |
| A-26 | N-[5-(aminosulfonyl)-3-methyl-1,3,4-triadiazol-2(3H)- |
| | ylidene]hydroxyacetamide |
| A-27 | Hydroxyethoxymethazolamide |
| A-28 | N-[5-(aminosulfonyl)-3-methyl-1,3,4-triadiazol-2(3H)- |
| | ylidene]hydroxyethoxyacetamide |
| A-29 | N-[5-(aminosulfonyl)-3-methyl-1,3,4-thiazol-2(3H)- |
| | ylidene]-2-[glycolyhydroxy]acetamide |

In yet another embodiment, the carbonic anhydrase inhibitor is selected from the class of dichlorophenamide carbonic anhydrase inhibitors represented by the general structure of Formula IIc shown below and possessing, by way of example and not limitation, the structures disclosed in Table 2c. Furthermore, dichlorophenamide carbonic anhydrase inhibitors useful in the practice of the present methods are described in U.S. Pat. Nos. 5,095,026 and 5,157,044, both of which are herein incorporated by reference in their entirety.

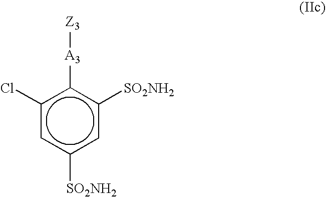

wherein:

Z₃ represents a water soluble carrier; and

A₃ is a moiety which is attached to the carbonic anhydrase inhibitor which allows it to still retain carbonic anhydrase inhibitory activity, but also form an enzymatically cleavable bond between A₃ and Z₃.

TABLE 2C

| Compound No. | Compound |
|---|---|
| A-30 | 4-hydroxy-5-chloro-m-benzenedisulfonamide |
| A-31 | 4-hydroxyethoxy-5-chloro-m-benzenedisulfonamide |
| A-32 | 4-hydroxyacetamido-5-chloro-M- |
| | benzenedisulfonamide |
| A-33 | 4-hydroxyethoxyacetamido-5-chloro-m- |
| | benzenedisulfonamide |
| A-34 | 4-amino-6-chloro-m-benzenedisulfonamides |
| A-35 | 4-hydroxyacetamido-6-chloro-m- |
| | benzenedisulfonamides |
| A-36 | 4-hydroxy-6-chloro-M-benzenedisulfonamides |
| A-37 | 4-hydroxyethoxy-6-chloro-m-benzenedisulfonamides |
| A-38 | 4-chloro-5-hydroxy-m-benzenedisulfonamides |
| A-39 | 4-chloro-5-hydroxyethoxy-m-benzenedisulfonamides |
| A-40 | 4-amino-5-chloro-m-benzenedisulfonamides |
| A-41 | 4-chloro-5-amino-m-benzenedisulfonamides |
| A-42 | 4-chloro-5-hydroxyacemido-m-benzenedisulfonamides |

In still another embodiment, the carbonic anhydrase inhibitor is selected from the class of methazolamide carbonic anhydrase inhibitors represented by the general structure of Formula III shown below and possessing, by way of example and not limitation, the structures disclosed in Table 3. Furthermore, methazolamide carbonic anhydrase inhibitors useful in the practice of the present methods are described in U.S. Pat. No. 5,104,887, both of which are herein incorporated by reference in their entirety.

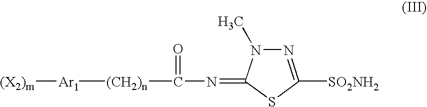

wherein:

n is an integer which is 0, 1, 2, 3,4, or 5;

X₂ is hydrogen, hydroxyl, hydroxylmethyl, 2-hydroxyethyl, or 2-hydroyethoxy;

Ar₁ is phenyl, pyridyl, or furanyl; and

m is an integer which is 0, 1, 2, 3, or 4.

TABLE 3

| Compound No. | Compound |
|---|---|
| A-43 | 6-[1-glucopyranosyl)oxyethoxy]-2- |
| | benzothiazolesulfonamide |
| A-44 | 2-ethoxycarbonylimino-3-methyl-delt⁴-1,3,4- |
| | thiadiazoline-5-sulfonamide |
| A-45 | 3-methyl-delta⁴-1,3,4-thiadiazoline-5-sulfonamide |
| A-46 | 2-[4-pyridylmethyloxycarbony]imino-3-methyl- |
| | delta⁴-1,3,4-thiadiazoline-5-sulfonamide |
| A-47 | 2-[4-hydroxymethylbenzyloxycarbonyl]imino-3-methyl- |
| | delta⁴-1,3,4-thiadiazoline-5-sulfonamide |
| A-49 | 2-[4-hydroxymethylphenylacetyl]imino-3-methyl- |
| | delta⁴-1,3,4-thiadiazoline-5-sulfonamide |
| A-50 | 2-benzyloxcarbonylimino-3-methyl-delta⁴-1,3,4- |
| | thiadiazoline-5-sulfonamide |
| A-51 | 2-ethoxycarbonylimino-3-methyl-delta4-1,3,4- |
| | thiadiazoline-5-sulfonamide |

In another embodiment, the carbonic anhydrase inhibitor is selected from the class of thiophene sulfonamide carbonic anhydrase inhibitors represented by the general structure of Formula IV shown below and possessing, by way of example and not limitation, the structures disclosed in Table 4. Furthermore, thiophene sulfonamide carbonic anhydrase inhibitors useful in the practice of the present methods are described in U.S. Pat. Nos. 5,153,192, 5,240,923, 5,378,703, and 5,620,970, all of which are herein incorporated by reference in their entirety.

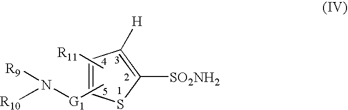

wherein:

R₉ is H, C₁₋₄ alkyl, C₂₋₄ alkyl substituted optionally with OH, halogen, C₁₋₄ alkoxy, or C(═O)R₁₅;

R₁₀ is H; C₁₋₈ alkyl; C₂₋₈ alkyl substituted with OH, NR₁₃R₁₄, halogen, C₁₋₄ alkoxy or C(═O)R₁₅; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₃ alkyl substituted with phenyl or heteroaryl which can be unsubstituted or substituted optionally with OH, (CH₂)ₙNR₁₃R₁₄, halogen, C₁₋₄ alkoxy, C₁₋₄ haloalkoxy, C(═O)R₁₅, S(═O)ₘ R₁₆ or SO₂N₁₃R₃R₁₄, wherein m is 0-2 and n is 0-2; C₂₋₄ alkoxy substituted optionally with NR₁₃R₁₄, halogen, C₁₋₄ alkoxy, or C(═O)R₁₅; phenyl, or heteroaryl, unsubstituted or substituted optionally with OH, (CH₂)ₙNR₁₃R₁₄, halogen, C₁₋₄ alkoxy, C₁₋₄ haloalkoxy, C(═O)R₁₅, S(═O)ₘ R₁₆ or SO₂NR₁₃R₁₄, wherein in is 0-2 and n is 0-2; provided that R₉ and R₁₀ cannot both be H; or R₉ and R₁₀ can be joined to form a saturated ring of 5 or 6 atoms selected from O, S, C or N which can be unsubstituted or substituted optionally on carbon with OH, NR₁₃R₁₄, halogen, C₁₋₄ alkoxy, C(═O)R₁₅, C₁₋₆ alkyl, C₁₋₆ alkyl substituted optionally with OH, NR₁₃R₁₄, halogen, C₁₋₄ alkoxy, C(═O)R₁₅ or on nitrogen with NR₁₃R₁₄, C₁₋₄ alkoxy, C(═O)R₁₅, C₁₋₆ alkyl or C₂₋₆ alkyl substituted optionally with OH, NR₁₃R₁₄, halogen, C₁₋₄ alkoxy or C(═O)R₁₅;

R₁₁ is H; halogen; C₁₋₄ alkyl; C₁₋₈ alkoxy; C₁₋₈ alkylthiol; C₂₋₈ alkoxy substituted optionally with OH, NR₁₃R₁₄, halogen, C₁₋₄ alkoxy or C(═O)R₁₅; C₁₋₄ alkyl substituted optionally with R₁₂; or R₉ and R₁₁ can be joined together with carbon atoms to form a ring of from 5 to 7 members in which said carbon atoms can be unsubstituted or substituted optionally with R₁₂;

R₁₂ is OH; C₁₋₄ alkyl unsubstituted or substituted optionally with OH, NR₁₃R₁₄, halogen, C₁₋₄ alkoxy or C(═O)R₁₅; C₁₋₄ alkoxy; C₂₋₄ alkoxy substituted optionally with OH, NR₁₃R₁₄, halogen, C₁₋₄ alkoxy or C(═O)R₁₅; NR₁₃R₁₄; phenyl, or heteroaryl, unsubstituted or substituted optionally with OH, (CH₂)ₙNR₁₃R₁₄, halogen, C₁₋₄ alkoxy, C₁₋₄ haloalkoxy, C(═O)R₁₅, S(═O)ₘ R₁₆ or SO₂NR₁₃R₁₄, wherein m is 0-2 and n is 0-2;

R₁₃ and R₁₄ are the same or different and are H; C₁₋₄ alkyl; C₂₋₄ alkyl substituted optionally with OH, halogen, C₁₋₄ alkoxy or C(═O)R₁₅; C₁₋₄ alkoxy; C₂₋₄ alkoxy substituted optionally with OH, halogen, C₁₋₄ alkoxy or C(═O)R₁₅; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₂ alkylC₃₋₅ cycloalkyl; or R₁₃ and R₁₄ can be joined to form a ring of 5 or 6 atoms selected from O, S, C or N which can be unsubstituted or substituted optionally on carbon with OH, (═O), halogen, C₁₋₄ alkoxy, C(═O)R₁₅, C₁₋₆ alkyl, C₁₋₆ alkyl substituted optionally with OH, halogen, C₁₋₄ alkoxy, C(═O)R₁₅ or on nitrogen with C₁₋₄ alkoxy, C(═O)R₁₅, S(═O)ₘ R₁₆, C₁₋₆ alkyl or C₂₋₆ alkyl substituted optionally with OH, halogen, C₁₋₄ alkoxy, C(═O)R₁₅ or on sulfur by (═O)ₘ, wherein m is 0-2;

R₁₅ is C₁₋₈ alkyl; C₁₋₈ alkyl substituted optionally with OH, NR₁₃R₁₄, halogen, C₁₋₄ alkoxy or C(═O)R₁₇; C₁₋₄ alkoxy; C₂₋₄ alkoxy substituted optionally with OH, NR₁₃R₁₄, halogen or C₁₋₄ alkoxy; or NR₁₃R₁₄;

R₁₆ is C₁₋₄ alkyl; C₂₋₄ alkyl substituted optionally with OH, NR₁₃R₁₄, halogen, C₁₋₄ alkoxy or C(═O)R₁₅; and

R₁₇ is C₁₋₄ alkyl; C₁₋₄ alkoxy; amino, C₁₋₃ alkylamino, or di-C₁₋₃ alkylamino; and G₁ is C(═O) or SO₂.

TABLE 4

| Compound No. | Compound |
|---|---|
| A-52 | 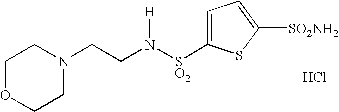 |
| | N-[2-(4-morpholinyl)ethyl]-2,5-thiophenedisulfonamide hydrochloride |
| A-53 | 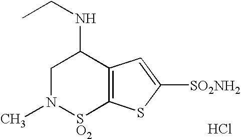 |
| | 4-ethylamino-3,4-dihydro-2-methyl-2H-thieno |
| | [3,2-e]-1,2-thiazine-6-sulfonamide 1,1 dioxide hydrochloride |
| A-54 | 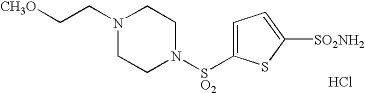 |
| | 5-[[4-(2-methoxyethyl)piperazinyl]sulfonyl]-2- |
| | thiophenesulfonamide hydrochloride |
| A-55 | 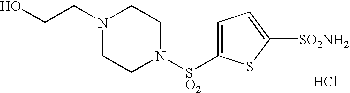 |
| | 5-[[4-12-hydroxyethyl)piperazinyl]sulfonyl]-2- |
| | thiophenesulfonamide hydrochloride |
| A-56 | 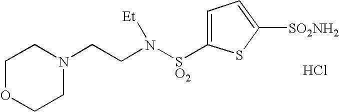 |
| | N-ethyl-N-[2-(4-morpholinyl)ethyl]-2,5- |
| | thiophenedisulfonamide hydrochloride |
| A-57 | 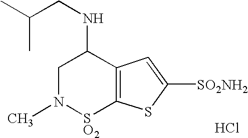 |
| | 3,4-dihydro-2-methyl-4-(2-methyl)propylamino-2H-thieno[3,2- |
| | e]-1,2-thiazine-6-sulfonamide 1,1-dioxide hydrochloride |
| A-58 | 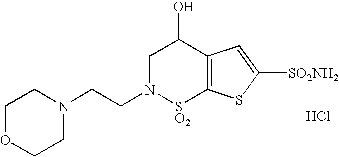 |
| | 3,4-dihydro-4-hydroxy-2-12-{4-morpholinyl)ethyl]-2H- |
| | thieno[3,2-e]-1,2-thiazine-6-sulfonamide 1,1-dioxide hydrochloride |
| A-59 | 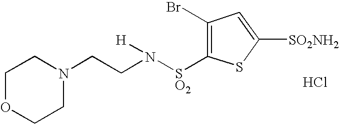 |
| | 4-bromo-5-[[2-(4-morpholinyl)ethyl]amino]sulfonyl-2- |
| | thiophenesulfonamide hydrochloride |
| A-60 | 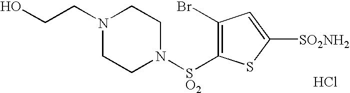 |
| | 4-bromo-5-[[4-(2-hydroxyethyl)-piperazinyl]sulfonyl]-2- |
| | thiphenesulfonamide hydrochloride |
| A-61 | 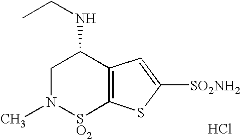 |
| | R-(+)-4-ethylamino-3,4-dihydro-2-methyl-2H-thieno[3,2-e]- |
| | 1,2-thiazine-6-sulfonamide 1,1-dioxide hydrochloride |
| A-62 | 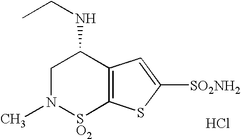 |
| | R-(+)-4-ethylamino-3,4-dihydro-2-methyl-2H-thieno[3,2-e]- |
| | 1,2-thiazine-6-sulfonamide 1,1-dioxide hydrochloride |
| A-63 | 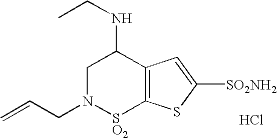 |
| | 2-allyl-4-ethylamino-3,4-dihydro-2H-thieno[3,2-e]-1,2-thiazine- |
| | 6-sulfonamide 1,1-dioxide hydrochloride |
| A-64 | 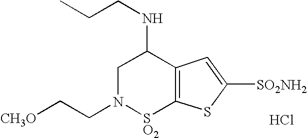 |
| | 3,4-dihydro-2-(2-methoxy)ethyl-4-propylamino-2H-thieno[3,2- |
| | e]-1,2-thiazine-6-sulfonamide 1,1-dioxide hydrochloride |
| A-65 | 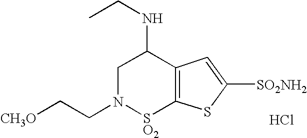 |
| | 4-ethylamino-3,4-dihydro-2-(2-methoxy)ethyl-2H-thieno[3,2- |
| | e]-1,2-thiazine-6-sulfonamide 1,1-dioxide hydrochloride |
| A-66 | 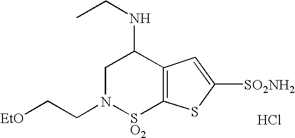 |
| | 2-(2-ethoxy)ethyl-4-ethylamino-3,4-dihydro-2H-thieno[3,2-e]- |
| | 1,2-thiazine-6-sulfonamide 1,1-dioxide hydrochloride |
| A-67 | 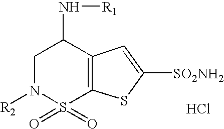 |
| A-68 | 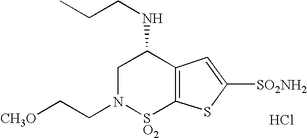 |
| | R-(+)-3,4-dihydro-2-(2-methoxy)ethyl-4-propylamino-2H- |
| | thieno[3,2-e]-1,2-thiazine-6-sulfonamide 1,1-dioxide hydrochloride |
| A-69 | 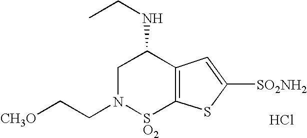 |
| | R-(+)-4-ethylamino-3,4-dihydro-2-(2-methoxy)ethyl-2H- |
| | thieno[3,2-e]-1,2-thiazine-6-sulfonamide 1,1-dioxide hydrochloride |
| A-70 | 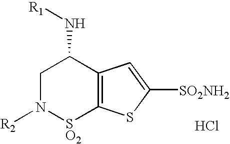 |
| A-71 | 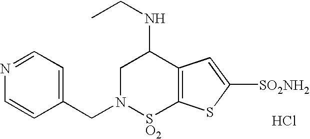 |
| | 4-ethylamino-3,4-dihydro-2-(4-pyridinyl)methyl-2H-thieno 3,2- |
| | e]-1,2-thiazine-6-sulfonamide 1,1-dioxide hydrochloride |
| A-72 | 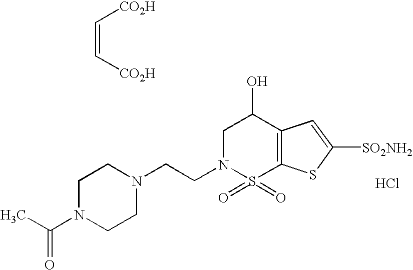 |
| | 2-[1-(4-acetyl-piperazinyl)]ethyl-3,4-dihydro-4-hydroxy-2H- |
| | thieno[3,2-e]-1,2-thiazine-6-sulfonamide-1,1-dioxide maleate |
| A-73 | 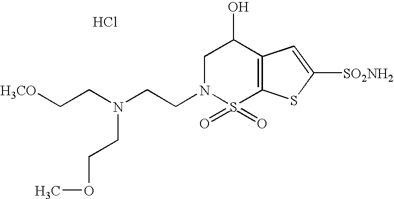 |
| | 3,4-dihydro-hydroxy-2-[2-(N,N-dimethoxyethyl)aminoethyl]- |
| | 2H-thieno-[3,2-e]-1,2-thiazine-6-sulfonamide-1,1-dioxide hydrochloride |
| A-74 | 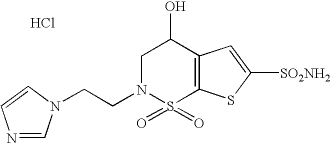 |
| | 3,4-dihydro-4-hydroxy-2,2-imidazol-1-yl)ethyl-2H-thieno[3,2- |
| | e]-1,2-thiazine-6-sulfonamide-1,1-dioxide hydrochloride |
| A-75 | 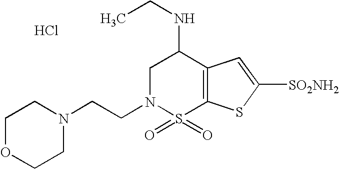 |
| | 4-ethylamino-3,4-dihydro-2-2-(4-morpholinyl)ethyl]-2H- |
| | thieno[3,2-e]-1,2-thiazine-6-sulfonamide-1,1-dioxide hydrochloride |
| A-76 | 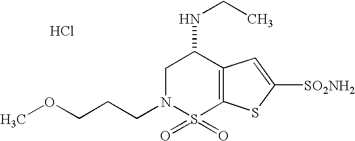 |
| | (−)-4-ethylamino-3,4-dihyro-2-(3-methoxy)propyl-2H- |
| | thieno[3,2-e]-1,2-thiazine-6-sulfonamide-1,1-dioxide hydrochloride |
| A-77 | 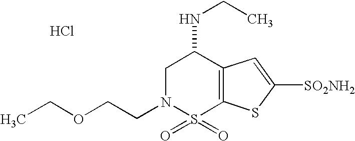 |
| | (+)-2-(2-ethoxy)ethyl-4-ethylamino-3,4-dihydro-2H-thieno[3,2- |
| | e]-1,2-thiazine-6-sulfonamide-1,1-dioxide hydrochloride |
| A-78 | 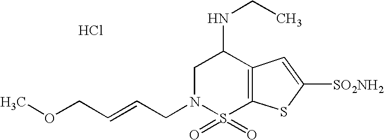 |
| | 4-ethylamino-3,4-dihydro-2-trans-(4-methoxy)-2-butenyl]-2H- |
| | thieno[3,2-e]-1,2-thiazine-6-sulfonamide-1,1-dioxide hydrochloride |
| A-79 | 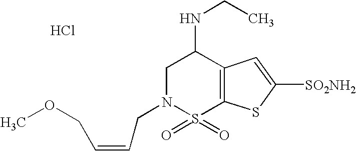 |
| | 4-ethylamino-3,4-dihydro-2-[cis-(4-methoxy)-2-butenyl]-2H- |
| | thieno[3,2-e]-1,2-thiazine-6-sulfonamide-1,1-dioxide hydrochloride |
| A-80 | 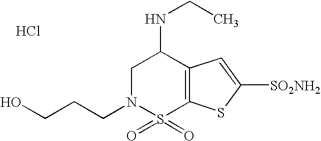 |
| | 4-ethylamino-3,4-dihydro-2-(3-hydroxy)propyl-2H-thieno[3,2- |
| | e]-1,2-thiazine-6-sulfonamide-1,1-dioxide hydrochloride |
| A-81 | 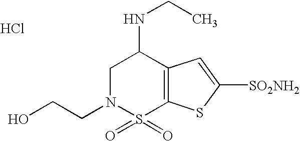 |
| | 4-ethylamino-3,4-dihydro-2-(2-hydroxy)ethyl-2H-thieno[3,2-e]- |
| | 1,2-thiazine-6-sulfonamide-1,1-dioxide hydrochloride |
| A-82 | 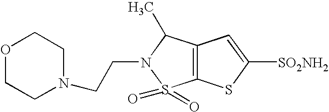 |
| | 2,3-dihydro-3-methyl-2-[2-(4-morpholinyl)ethyl]-thieno13,2- |
| | d]-isothiazole-5-sulfonamide-1,1-dioxide |
| A-83 | 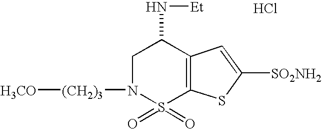 |
| | (+)-4-ethylamino-3,4-dihydro-2-(3-methoxy)propyl-2H- |
| | thieno[3,2-e]-1,2-thiazine-6-sulfonamide-1,1-dioxide hydrochloride |
| A-84 | 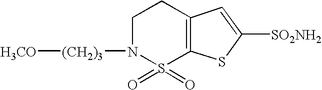 |
| | 3,4-dihydro-2-(3-methoxypropyl)-2H-thieno[3,2-e]-1,2- |
| | thiazine-6-sulfonamide-1,1-dioxide sodium salt |
| A-85 | 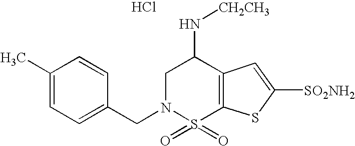 |
| | 4-ethylamino-3,4-dihydro-2-(4-methylphenyl)methyl-2H- |
| | thieno[3,2-e]-1,2-thiazine-6-sulfonamide-1,1-dioxide hydrochloride |
| A-86 | 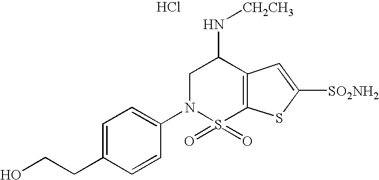 |
| | 4-ethylamino-3,4-dihydro-2-[4-(2-hydroxyphenyl)phenyl]-2H- |
| | thieno[3,2-e]-1,2-thiazine-6-sulfonamide 1,1-dixoide hydrochloride |
| A-87 | 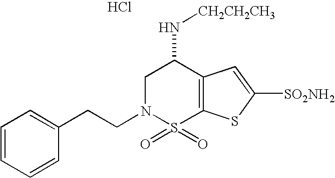 |
| | R-(+)-3,4-dihydro-2-(2-phenylethyl)-4-propylamino-2H- |
| | thieno[3,2-e]-1,2-thiazine-6-sulfonamide 1,1-dioxide hydrochloride |
| A-88 | 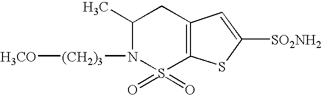 |
| | 3,4-dihydro-2-(3-methoxypropyl)-3-methyl-2H-thieno[3,2-e]- |
| | 1,2-thiazine-6-sulfonamide 1,1-dioxide |
| A-89 | 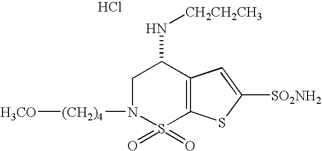 |
| | R-(+)-3,4-dihydro-2-(4-methoxybutyl)-4-propylamino-2H- |
| | thieno[3,2-e]-1,2-thiazine-6-sulfonamide 1,1-dioxide hydrochloride |
| A-90 | 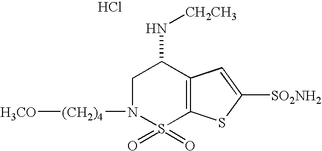 |
| | R-(+)-4-ethylamino-3,4-dihydro-2-(4-methoxybutyl)-2H- |
| | thieno[3,2-e]-1,2-thiazine-6-sulfonamide 1,1-dioxide hydrochloride |
| A-91 | 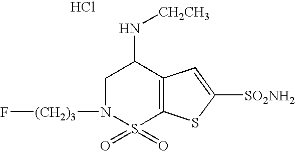 |
| | 4-ethylamino-2-(3-fluoropropyl)3,4-dihydro-2H-thieno[3,2-e]- |
| | 1,2-thiazine-6-sulfonamide 1,1-dioxide hydrochloride |
| A-92 | 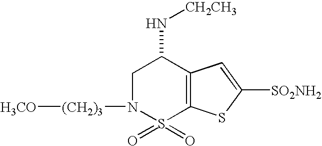 |
| | R-(−)-4-ethoxy-3,4-dihydro-2-(3-methoxypropyl)-2H- |
| | thieno[3,2-e]-1,2-thiazine-6-sulfonamide 1,1-dioxide hydrochloride |
| A-93 | 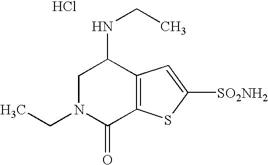 |
| | 6-ethyl-4-ethylamino-4,5,6,7-tetrahydro-7-oxo-thieno[2,3- |
| | b]pyridine-2-sulfonamide hydrochloride |
| A-94 | 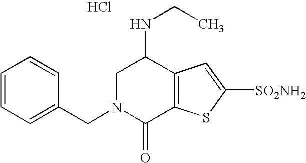 |
| | 4-ethylamino-4,5,6,7-tetrahydro-7-oxo-6-(phenylmethyl)- |
| | thieno[2,3-b]pyridine-2-sulfonamide hydrochloride |
| A-95 | 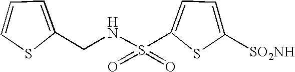 |
| | N-(2-thienyl)methyl-2,5-thiophenedisulfonamide |
| A-96 | 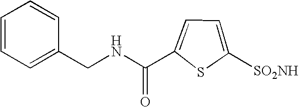 |
| | N-(phenylmethyl)-5-(aminosulfonyl)-thiophene-2-carboxamide |

In yet another embodiment, the carbonic anhydrase inhibitor is selected from the class of methazolamide carbonic anhydrase inhibitors represented by the general structure of Formula V shown below and possessing, by way of example and not limitation, the structures disclosed in Table 5. Furthermore, methazolamide carbonic anhydrase inhibitors useful in the practice of the present methods are described in U.S. Pat. No. 5,225,424, which is herein incorporated by reference in its entirety.

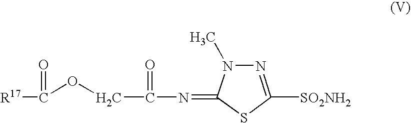

In one embodiment, R¹⁷ is C₁₋₈. In another embodiment, R¹⁷ is C₁₋₄. In still another embodiment, R¹⁷ is methyl.

TABLE 5

| Compound No. | Compound |
|---|---|
| A-97 | N-[5-(aminosulfonyl)-3-methyl]-1,3,4-thiadiazol-2(3H)- |
| | ylidene]-2-acetyloxyacetamide |
| A-98 | 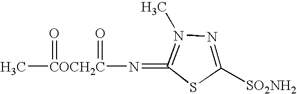 |
| | N-[5-(aminosulfonyl)-3-methyl]-1,3,4-thiadiazol-2(3H)- |
| | ylidene]-2-acetyloxyacetamide |

In still another embodiment, the carbonic anhydrase inhibitor is selected from the class of thienothiazine sulfonamide carbonic anhydrase inhibitors represented by the general structure of Formula VI shown below and possessing, by way of example and not limitation, the structures disclosed in Table 6. Furthermore, thienothiazine sulfonamide carbonic anhydrase inhibitors useful in the practice of the present methods are described in U.S. Pat. Nos. 5,344,929 and 5,424,448, both of which are herein incorporated by reference in their entirety.

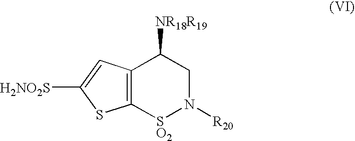

wherein:

R₁₈ and R₁₉ are H or C₁₋₄ alkyl;

R₂₀ is C₁₋₆ alkyl, CH₂(CH₂)ₙOR₂₁, where n is 1-4; and

R₂₁ is CH₃, (CH₂)ₙCH₃ where n is 1-4, or (CH₂)ₙAr₂ where Ar₂ is unsubstituted phenyl, 3-methoxyphenyl, or 4-methoxyphenyl and n is 1 or 2.

TABLE 6

| Compound No. | Compound |
|---|---|
| A-99 | 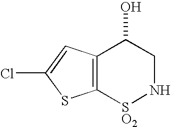 |
| | (S)-3,4-dihydro-6-chloro-4-hydroxy-2H-thieno[3,2-e]- |
| | 1,2-thiazine-1,1-dioxide |
| A-100 | 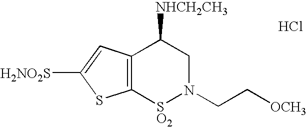 |
| | (R)-3,4-dihydro-4-ethylamino-2-(2-methoxyethyl)-2H- |
| | thieno[3,2-e]-1,2-thiazine-6-sulfonamide-1,1-dioxide |
| | hydrochloride |
| A-101 | 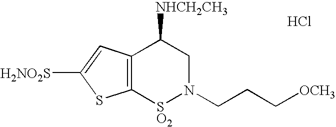 |
| | (R)-3,4-dihydro-4-ethylamino-2-(3-methoxypropyl)-2H- |
| | thieno[3,2-e]-1,2-thiazine-6-sulfonamide-1,1-dioxide |
| A-102 | 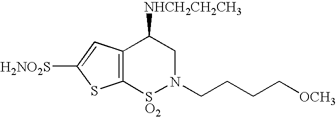 |
| | (R)-3,4-dihydro-2-(4-methoxybutyl)-4-propylamino-2H- |
| | thieno[3,2-e]-1,2-thiazine-6-sulfonamide-1,1-dioxide |
| A-103 | 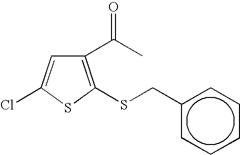 |
| A-104 | 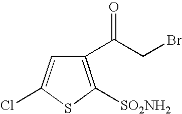 |

In a further embodiment, the carbonic anhydrase inhibitor is selected from the class of thienothiazine sulfonamide carbonic anhydrase inhibitors represented by the general structure of Formula VII shown below and possessing, by way of example and not limitation, the structures disclosed in Table 7. Furthermore, thienothiazine sulfonamide carbonic anhydrase inhibitors useful in the practice of the present methods are described in U.S. Pat. No. 5,464,831, which is herein incorporated by reference in its entirety.

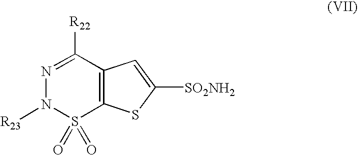

wherein:

R₂₂ is H, C₁₋₆ alkyl unsubstituted or substituted optionally with OH, C₁₋₄ alkoxy, NR₂₄R₂₅, OC(═O)R₂₆ or C(═O)R₂₆;

R₂₃ is H; C₁₋₈ alkyl; C₁₋₈ alkyl substituted with OH, NR₂₄R₂₅, halogen, C₁₋₄ alkoxy, C₂₋₄ alkoxy, C₁₋₄ alkoxy, OC(═O)R₂₆, S(═O)ₘ R₂₈, or C(═O)R₂₆; C₃₋₇ alkenyl unsubstituted or substituted optionally with OH, NR₂₄R₂₅, or C₁₋₄ alkoxy; C₃₋₇ alkynyl unsubstituted or substituted optionally with OH, NR₂₄R₂₅, or C₁₋₄ alkoxy; C₀₋₃ alkyl substituted with R₂₇ which can be unsubstituted or substituted optionally with C₁₋₃ alkyl, C₁₋₃ haloalkyl, OH, (CH₂)ₙ NR₂₄R₂₅, halogen, C₁₋₄ alkoxy, C₁₋₄ haloalkoxy, OC(═O)R₂₆, C(═O)R₂₆, S(═O)ₘ R₂₈ or SO₂NR₂₄R₂₅, wherein m is 0-2 and n is 0-2;

R₂₄ and R₂₅ are independently H; C₁₋₈ alkyl; C₂₋₄ alkyl substituted optionally with OH, halogen, C₁₋₄ alkoxy or C(═O)R₂₆; OH; C₁₋₄ alkoxy; C₂₋₄ alkoxy substituted optionally with OH, halogen, C₁₋₄ alkoxy or C(═O)R₂₆; or R₂₄ and R₂₅ can be joined to form a ring of 5 or 6 atoms selected from O, S, C or N which can be unsubstituted or substituted optionally on carbon with OH, (═O), halogen, C₁₋₄ alkoxy, C(═O)R₂₆, C₁₋₆ alkyl, C₁₋₆ alkyl substituted optionally with OH, halogen, C₁₋₄ alkoxy, C(═O)R₂₆ or on nitrogen with C₁₋₄ alkoxy, C(═O)R₂₆, S(═O)ₘ R₂₈, C₁₋₆ alkyl or C₂₋₆ alkyl substituted optionally with OH, halogen, C₁₋₄ alkoxy, C(═O)R₂₆ or on sulfur by (═O)ₘ, where m is 0-2;

R₂₆ is C₁₋₈ alkyl; C₁₋₄ alkyl substituted optionally with OH, NR₂₄R₂₅, halogen, C₁₋₄ alkoxy or C(═O)R₂₉; C₁₋₄ alkoxy; C₂₋₄ alkoxy substituted optionally with OH, NR₂₄R₂₅, halogen or C₁₋₄ alkoxy; or NR₂₄R₂₅;

R₂₇, is a monocyclic ring system of 5 or 6 atoms composed of C, N, O or S, such as benzene, furan, thiophene, pyrrole, pyrazole, imidazole, triazole, tetrazole, oxazole, isoxazole, isothiazole, thiazole, thiadiazole, pyridine pyrimidine, pyridazine, and pyrazine;

R₂₈ is C₁₋₄ alkyl; C₂₋₄ alkyl substituted optionally with OH, NR₂₄R₂₅, C₁₋₄ alkoxy or C(═O)R₂₆; R₂₇ which can be unsubstituted or substituted optionally with OH, (CH₂)ₙ NR₂₄R₂₅, halogen, C₁₋₄ alkoxy, C₁₋₄ haloalkoxy, C(═O)R₂₆, S(═O)ₘ C₁₋₄ alkyl or SO₂ NR₂₄R₂₅; wherein m is 0-2 and n is 0-2; and

R₂₉ is C₁₋₄ alkyl; C₁₋₄ alkoxy; amino, C₁₋₃ alkylamino, of di-C₁₋₃ alkylamino.

TABLE 7

| Compound No. | Compound |
|---|---|
| A-105 | 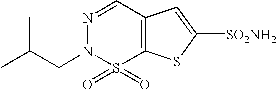 |
| | 2-(2-methylpropyl)-2H-thieno[3,2-e]-1,2,3-thiadiazine-6- |
| | sulfonamide 1,1-dioxide |
| A-106 | 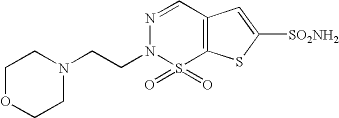 |
| | 2-[2-(4-morpholinyl)ethyl]-2H-thieno[3,2-e]-1,2,3- |
| | thiadiazine-6-sulfonamide 1,1-dioxide |
| A-107 | 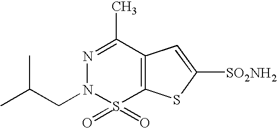 |
| | 4-methyl-2-(2omethylpropyl)-2H-thieno[3,2-e]-1,2,3- |
| | thiadiazine-6-sulfonamide 1,1-dioxide |
| A-108 | 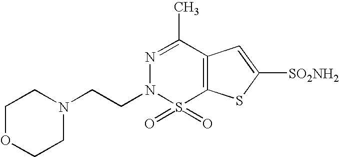 |
| | 2-[2-(4-morpholinyl)ethyl]-4-methyl-2H-thieno[3,2-e]- |
| | 1,2,3-thiadiazine-6-sulfonamide 1,1-dioxide |

In another embodiment, the carbonic anhydrase inhibitor is selected from the class of thienothiazine sulfonamide carbonic anhydrase inhibitors represented by the general structure of Formula VIII shown below and possessing, by way of example and not limitation, the structures disclosed in Table 8. Furthermore, thienothiazine sulfonamide carbonic anhydrase inhibitors useful in the practice of the present methods are described in U.S. Pat. No. 5,510,347, which is herein incorporated by reference in its entirety.

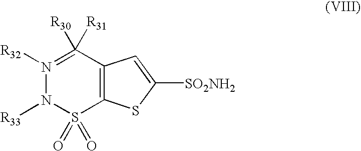

wherein:

R₃₀ is H or C₁₋₂ alkyl;

R₃₁ is H; C₁₋₆ alkyl unsubstituted or substituted optionally with OH, C₁₋₄ alkoxy, NR₃₄R₃₅, OC(═O)R₃₆ or C(═O)R₃₆;

R₃₂ is H; C₁₋₆ alkyl; C₂₋₄ alkyl substituted with OH, NR₃₄R₃₅, halogen, C₁₋₄ alkoxy, C₂₋₄ alkoxy, C₁₋₄ alkoxy, OC(═O)R₃₆, S(═O)ₘR₃₇, or C(═O)R₃₆; C(═O)R₃₆;

R₃₃ is H; C₁₋₈ alkyl; C₁₋₈ alkyl substituted with OH, NR₃₄R₃₅, halogen, C₁₋₄ alkoxy, C₂₋₄ alkoxy, C₁₋₄ alkoxy, OC(═O)R₃₆, S(═O)ₘR₃₇, or C(═O)R₃₆; C₁₋₇ alkenyl unsubstituted or substituted optionally with OH, NR₃₄R₃₅, or C₁₋₄ alkoxy; C₃₋₇ alkynyl unsubstituted or substituted optionally with OH, NR₃₄R₃₅, or C₁₋₄ alkoxy; C₁₋₃ alkyl substituted with R₃₇ which can be unsubstituted or substituted optionally with C₁₋₃ alkyl, C₁₋₃ haloalkyl, OH, (CH₂)ₙ NR₃₄R₃₅, halogen, C₁₋₄ alkoxy, C₁₋₄ haloalkoxy, OC(═O)R₃₆, C(═O)R₃₆, S(═O)ₘ R₃₈ or SO₂ NR₃₄R₃₅, wherein m is 0-2 and n is 0-2;

R₃₄ and R₃₅ are H; C₁₋₈ alkyl; C₂₋₄ alkyl substituted optionally with OH, halogen, C₁₋₄ alkoxy or C(═O)R₃₆; OH; C₁₋₄ alkoxy; C₂₋₄ alkoxy substituted optionally with OH, halogen, C₁₋₄ alkoxy or C(═O)R₃₆; or R₃₄ and R₃₅ can be joined to form a ring of 5 or 6 atoms selected from O, S, C or N which can be unsubstituted or substituted optionally on carbon with OH, (═O), halogen, C₁₋₄ alkoxy, C(═O)R₃₆, C₁₋₆ alkyl, C₁₋₆ alkyl substituted optionally with OH, halogen, C₁₋₄ alkoxy, C(═O))R₃₆ or on nitrogen with C₁₋₄ alkoxy, C(═O)R₃₆, S(═O)ₘ R₃₈, C₁₋₆ alkyl or C₂₋₆ alkyl substituted optionally with OH, halogen, C₁₋₄ alkoxy, C(═O)R₃₆ or on sulfur by (═O)ₘ, wherein m is 0-2;

R₃₆ is C₁₋₈ alkyl; C₁₋₄ alkyl substituted optionally with OH, NR₃₄R₃₅, halogen, C₁₋₄ alkoxy or C(═O)R₃₉; C₁₋₄ alkoxy; C₂₋₄ alkoxy substituted optionally with OH, NR₃₄R₃₅, halogen or C₁₋₄ alkoxy; or NR₃₄R₃₅;

R₃₇ is a monocyclic ring system of 5 or 6 atoms composed of C, N, O or S, such as benzene, furan, thiophene, pyrrole, pyrazole, imidazole, triazole, tetrazole, oxazole, isoxazole, isothiazole, thiazole, thiadiazole, pyridine pyrimidine, pyridazine, and pyrazine, where R₃₇ can be unsubstituted or substituted optionally with OH, (CH₂)ₙ NR₃₄R₃₅, halogen, C₁₋₄ alkoxy, C₁₋₄ haloalkoxy, C(═O)R₃₆, S(═O)ₘ C₁₋₄ alkyl or SO₂ NR₃₄R₃₅; wherein m is 0-2 and n is 0-2;

R₃₈ is C₁₋₄ alkyl; C₂₋₄ alkyl substituted optionally with OH, NR₃₄R₃₅, C₁₋₄ alkoxy or C(═O)R₃₆; and

R₃₉ is C₁₋₄ alkyl; C₁₋₄ alkoxy; amino, CIl3 alkylamino, of di-C₁₋₃ alkylamino.

TABLE 8

| Compound No. | Compound |
|---|---|
| A-109 | 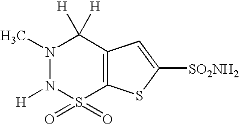 |
| | 3,4-dihydro-2H-thieno[3,2-e]-1,2,3-thiadiazine-3-methyl- |
| | 6-sulfonamide 1,1-dixoide |

In still another embodiment, the carbonic anhydrase inhibitor is selected from the class of sulfonamide carbonic anhydrase inhibitors represented by the general structure of Formula VIIII shown below and possessing, by way of example and not limitation, the structures disclosed in Table 9. Furthermore, sulfonamide carbonic anhydrase inhibitors useful in the practice of the present methods are described in U.S. Pat. No. 5,538,966, which is herein incorporated by reference in its entirety.

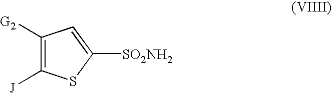

wherein G₂, J and the two atoms of the thiophene ring to which they are attached form a six-membered ring chosen from:

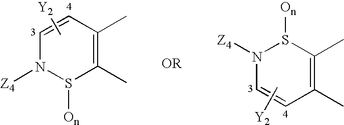

wherein:

Z₄ is Z₄ₐ, Z₄ₐ is C₁₋₈ alkyl; C1-3 alkyl-C3-6 cycloalkyl; CH₂ C(═O)R₄₆; CH₂ C(═O)NR₄₁R₄₂; CH₂CN; C₂₋₈ alkyl substituted with one or more of hydroxyl, C₁₋₄ alkoxy, C₂₋₄ alkoxy-C₁₋₄ alkoxy, OC(═O)R₄₀, N(R₄₁)C(═O)R₄₀, halogen, CN, NR₄₁R₄₂, SOₙR₄₃ or C(═O)R₄₄, C₁₋₄ alkyl substituted with an aromatic group chosen from phenyl or Q either of which can be unsubstituted or substituted with one or more of C₁₋₄ alkyl, C₁₋₄ alkoxy, hydroxy, halogen, nitrile, NR₄₁R₄₂, SOₙR₄₃, C(═O)R₄₄ or C₁₋₄ alkyl which is substituted with hydroxy, NR₁₄R₄₂, halogen, CO₂R₄₀ or C₁₋₃ alkoxy; C₃₋₈ alkenyl unsubstituted or substituted with hydroxyl, C₁₋₄ alkoxy or NR₄₁R₄₂; C₃₋₈ alkynyl unsubstituted or substituted with hydroxyl, C₁₋₄ alkoxy or NR₄₁R₄₁; and if Z₄ is Z_{4b}, Z_{4b} is an aromatic group chosen from phenyl or Q either of which can be unsubstituted or substituted with one or more of C₁₋₄ alkyl, C₁₋₄ alkoxy, hydroxy halogen, nitrile, NR₄₁R₄₂, SOₙR₄₃, C(═O)R₄₄, or C₁₋₄ alkyl which is substituted with hydroxy, NR₄₁R₄₂, halogen or C₁₋₃ alkoxy;

Y₂ is hydrogen; C₁₋₈ alkyl; C₁₋₆ alkyl substituted with one or more of hydroxyl, C₁₋₄ alkoxy, C₂₋₄ alkoxy-C₁₋₄ alkoxy, OC(═O)R₄₀, N(R₄₁)C(═O)R₄₀, halogen, CN, NR₄₁R₄₂, SOₙ R₄₃, or C(═O)R₄₄; C₁₋₄ alkyl substituted with an aromatic group chosen from phenyl or Q either of which can be unsubstituted or substituted with one or more of C₁₋₄ alkyl, C₁₋₄ alkoxy, hydroxy, halogen, nitrile, NR₄₁R₄₂, SOₙR₄₃, C(═O)R₄₄ or C₁₋₄ alkyl which is substituted with hydroxy, NR₄₁R₄₂, halogen, CO₂R₄₀ or C₁₋₃ alkoxy; C₃₋₈ alkenyl unsubstituted or substituted with hydroxyl, C₁₋₄ alkoxy or NR₄₁R₄₂; C₃₋₈ alkynyl unsubstituted or substituted with hydroxyl, C₁₋₄ alkoxy or NR₄₁R₄₂;

R₄₀ is C₁₋₆ alkyl; C₁₋₆ alkyl substituted with hydroxyl, halogen, C₁₋₄ alkoxy, NR₄₁R₄₂ or C(═O)R₄₄; phenyl which can be unsubstituted or substituted with one or more of C₁₋₄ alkyl, alkoxy, hydroxy or halogen;

R₄₁ and R₄₂ are independently chosen from hydrogen; C₁₋₄ alkyl; CH₂ CN; C₁₋₃ alkyl-C₃₋₆ cycloalkyl; C₃₋₈ cycloalkyl; C₂₋₄ alkyl substituted with hydroxyl, halogen, CN, C₁₋₄ alkoxy or C(═O)R₄₄; hydroxyl; C₁₋₄ alkoxy; C₂₋₄ alkoxy substituted with hydroxyl, NR₄₁R₄₂, halogen or C₁₋₄ alkoxy; C₃₋₈ alkenyl unsubstituted or substituted with hydroxy, or C₁₋₄ alkoxy; C₃₋₈ alkynyl unsubstituted or substituted with hydroxyl, or C₄ alkoxy; or further R₄₁ and R₄₂ together with the nitrogen atom to which they are attached can be incorporated into a saturated heterocyclic ring of 5 to 8 atoms which may include a second heteroatom selected from O, S or N, such as pyrrolidine, oxazolidine, morpholine, thiomorpholine, thiomorpholine 1,1-dioxide, piperazine, 2-oxa-5-azabicyclo[2.2.1]heptane, 2-oxa-5-azabicyclo[3.2.1]octane, thiazolidine, or thiazolidine 1,1-dioxide, which can be unsubstituted or substituted on carbon with hydroxyl, (═O), halogen, C₁₋₄ alkoxy, C(═O)R₄₄, C₁₋₄ alkyl, C₁₋₄ alkyl substituted with hydroxyl, halogen, C₁₋₄ alkoxy, C(═O)R₅, or on nitrogen with C₁₋₄ alkoxy, C(═O)R₄₄, SOₙ R₄₃, C₁₋₄ alkyl or C₁₋₄ alkyl substituted with hydroxyl, halogen, C₁₋₄ alkoxy or C(═O)R₄₄;

R₄₃ is C₁₋₄ alkyl; C₂₋₄ alkyl substituted with hydroxyl, halogen, NR₄₁R₄₂ or C₁₋₃ alkoxy;

R₄₄ is C₁₋₆ alkyl; C₁₋₆ alkyl substituted with hydroxyl, halogen, SOₙR₄₃, C₁₋₄ alkoxy, NR₄₁R₄₂ or C(═O)R₄₅; C₁₋₄ alkyl substituted with an aromatic group chosen from phenyl or Q either of which can be unsubstituted or substituted with one or more of C₁₋₄ alkyl, C₁₋₄ alkoxy, hydroxy, halogen, nitrile, NR₄₁R₄₂, SOₙR₄₃ or C₁₋₄ alkyl which is substituted with hydroxy, NR₄₁R₄₂, halogen or C₁₋₃ alkoxy; hydroxyl; C₁₋₄ alkoxy; C₂₋₄ alkoxy substituted with hydroxyl, NR₄₂, halogen or C₁₋₄ alkoxy; NR₄₁R₄₂;

R₄₅ is C₁₋₄ alkyl; C₁₋₄ alkoxy; amino; C₁₋₃ alkylamino; (C₁₋₃ alkyl)2 amino;

R₄₆ is hydroxyl, C₁₋₄ alkoxy, C₁₋₄ alkoxy substituted with hydroxyl, NR₄₁R₄₂ or C₁₋₄ alkoxy;

n is 0, 1, or 2; and

Q is a monocyclic five or six membered heterocyclic ring system wherein one or more of the heteroatoms nitrogen, oxygen and/or sulfur are incorporated into the ring, such as thiophene, furan, pyrrole, pyrazole, imidazole, triazole, tetrazole, oxazole, isoxazole, isothiazole, thiazole, thiadiazole, pyridine, pyrimidine, pyridazine, and pyrazine.

TABLE 9

| Compound No. | Compound |
|---|---|
| A-110 | 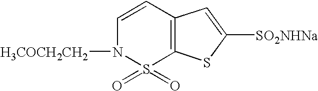 |
| | 2-(2-methoxyethyl)-2H-thieno[3,2-e]-1,2-thiazine-6- |
| | sulfonamide 1,1-dioxide sodium salt |
| A-111 | 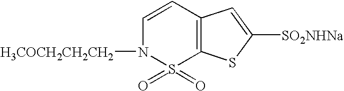 |
| | 2-(2-methoxypropyl)-2H-thieno[3,2-e]-1,2-thiazine-6- |
| | sulfonamide 1,1-dioxide sodium salt |
| A-112 | 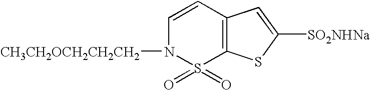 |
| | 2-(3-ethoxypropyl)-2H-thieno[3,2-e]-1,2-thiazine-6- |
| | sulfonamide 1,1-dioxide |
| A-113 | 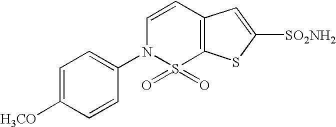 |
| | 2-(4-methoxyphenyl)-2H-thieno[3,2-e]-1,2-thiazine-6- |
| | sulfonamide 1,1-dioxide |
| A-114 | 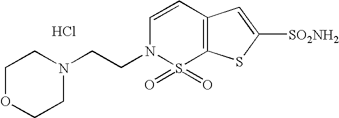 |
| | 2-[2-(4-morpholinyl)ethyl]-2H-thieno[3,2-e]-1,2-thiazine-6- |
| | sulfonamide 1,1-dioxide hydrochloride |
| A-115 | 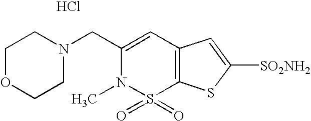 |
| | 2-methyl-3-(4-morpholinylmethyl)-2H-thieno[3,2-e]-1,2- |
| | thiazine-6-sulfonamide 1,1-dioxide hydrochloride |
| A-116 | 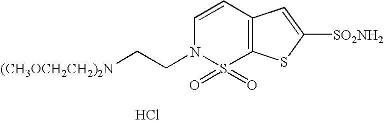 |
| | 2-[2-[bis(2-methoxyethyl)amino]ethyl]-2H-thieno[3,2-e]-1,2- |
| | thiazine-6-sulfonamide 1,1-dioxide hydrochloride |
| A-117 | 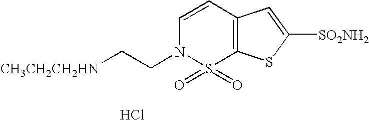 |
| | 2-[2-(propylamino)ethyl]-2H-thieno[3,2-e]-1,2-thiazine-6- |
| | sulfonamide 1,1-dioxide hydrochloride |
| A-118 | 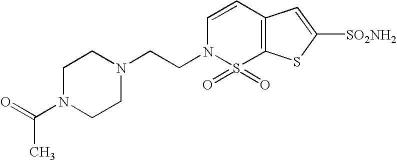 |
| | 2[2-[4-acetyl-(1-piperazinyl)]-2H-thieno[3,2-e]-1,2-thiazine-6- |
| | sulfonamide 1,1-dioxide |
| A-119 | 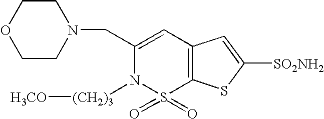 |
| | 2-(3-methoxypropyl)-3-(4-morpholinylmethyl)-2H-thieno[3,2- |
| | e]-1,2-thiazine-6-sulfonamide 1,1-dioxide hydrochloride |
| A-120 | 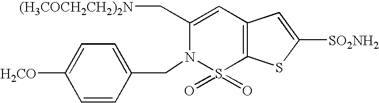 |
| | 3-[[bis(2-methoxyethyl)amino]methyl]-2-(4- |
| | methoxyphenylmethyl)-2H-thieno[3,2-e]-1,2-thiazine-6-sulfonamide 1,1-dioxide |
| A-121 | 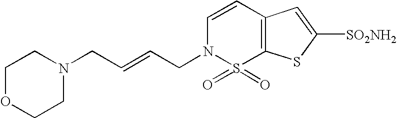 |
| | 2-[4-(4-morpholinyl)-2-butenyl]-2H-thieno[3,2-e]-1,2-thiazine- |
| | 6-sulfonamide 1,1-dioxide hydrochloride |
| A-122 | 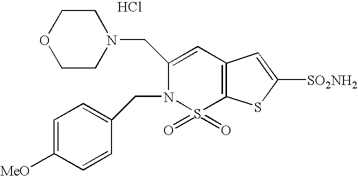 |
| | 2-(4-methoxyphenylmethyl)-3-(4-morpholinylmethyl)-2H- |
| | thieno[3,2-e]-1,2-thiazine-6-sulfonamide 1,1-dioxide hydrochloride |
| A-123 | 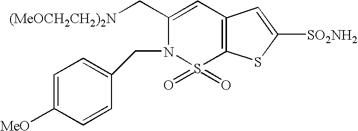 |
| | 3-[[bis(2-methoxyethyl)amino]methyl]-2-(4-methoxyphenyl)- |
| | 2H-thieno[3,2-e]-1,2-thiazine-6-sulfonamide 1,1-dioxide |
| A-124 | 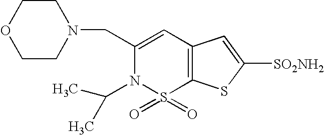 |
| | 2-(1-methylethyl)-3-(4-morpholinylmethyl)-2H-thieno[3,2-e]- |
| | 1,2-thiazine-6sulfonamide 1,1-dioxide |
| A-125 | 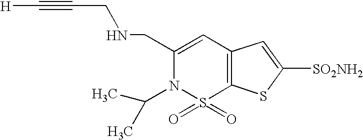 |
| | 2-(1-methylethyl)-3-[(2-propynylamino)methyl]-2H-thieno[3,2- |
| | e]-1,2-thiazine-6sulfonamide 1,1-dioxide |
| A-126 | 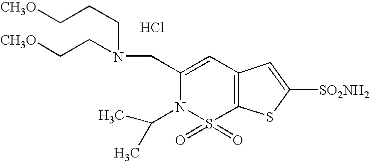 |
| | 2-(1-methylethyl)-3-[[(2-methoxyethyl)(3-methoxypropyl)amino]methyl]- |
| | 2H-thieno[3,2-e]-1,2-thiazine-6-sulfonamide 1,1-dioxide hydrochloride |
| A-127 | 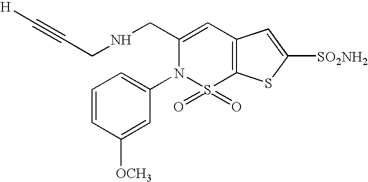 |
| | 2-(3-methoxyphenyl)-3-[(2-propynylamino)methyl]-2H- |
| | thieno[3,2-e]-1,2-thiazine-6-sulfonamide 1,1-dioxide |
| A-128 | 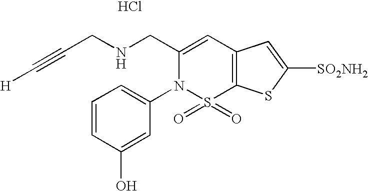 |
| | 2-(3-hydroxyphenyl)-3-[(2-propynylamino)methyl]-2H- |
| | thieno[3,2-e]-1,2-thiazine-6-sulfonamide 1,1-dioxide hydrochloride |
| A-129 | 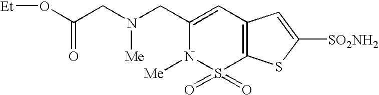 |
| | N-[[6-(aminosulfonyl)-2-methyl-2H-thieno[3,2-e]-1,2-thiazine- |
| | 3-yl]methyl]-N-methyl-glycine ethyl ester S¹,S¹-dioxide |
| A-130 | 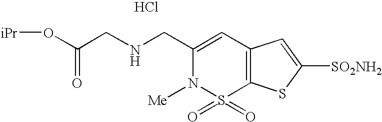 |
| | N-[[6-(aminosulfonyl)-2-methyl-2H-thieno[3,2-e]-1,2-thiazin- |
| | 3-yl]methyl]-glycine 2-methylethyl ester S¹,S¹-dioxide hydrochloride |
| A-131 | 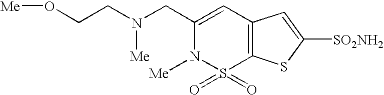 |
| | 3-[[(2-methoxyethyl)methylamino]methyl]-2-methyl-2H- |
| | thieno[3,2-e]-1,2-thiazine-6-sulfonamide 1,1-dioxide |
| A-132 | 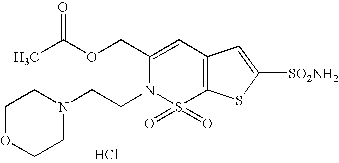 |
| | 3-[(acetyloxy)methyl]-2-[2-(4-morpholinyl)ethyl]-2H- |
| | thieno[3,2-e]-1,2-thiazine-6-sulfonamide 1,1-dioxide hydrochloride |
| A-133 | 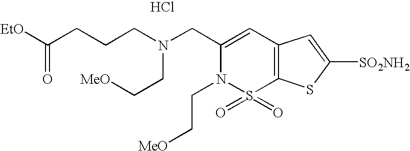 |
| | ethyl 4-[(2-methoxyethyl)[[6-(aminosulfonyl)-2-(2-methoxyethyl)-2H-thieno[3,2- |
| | e]-1,2-thiazin-2-yl]methyl]amino]butanoate S¹,S¹-dioxide hydrochloride |
| A-134 | 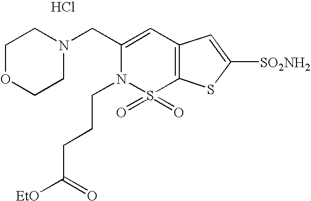 |
| | 6-(aminosulfonyl)-3-(4-morpholinylmethyl)-2H-thieno[3,2-e]- |
| | 1,2-thiazine-2-butanoic acid 1,1-dioxide ethyl ester |
| A-135 | 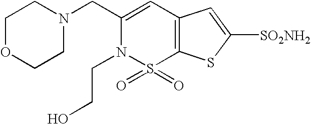 |
| | 2-(2-hydroxyethyl)-3-(4-morpholinylmethyl)-2H-thieno[3,2-e]- |
| | 1,2-thiazine-6-sulfonamide 1,1-dioxide |
| A-136 | 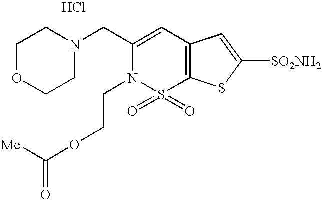 |
| | 2-[2-(acetyloxy)ethyl9 -3-(4-morpholinylmethyl)-H-thieno[3,2- |
| | e]-1,2-thiazine-6-sulfonamide 1,1-dioxide hydrochloride |
| A-137 | 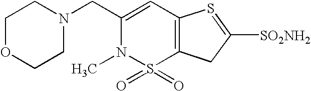 |
| | 2-methyl-3-(4-morpholinylmethyl)-2H-thieno[2,3-e]-1,2- |
| | thiazine-6-sulfonamide 1,1-dioxide |

In yet another embodiment, the carbonic anhydrase inhibitor is selected from the class of thiophene sulfonamide carbonic anhydrase inhibitors represented by the general structure of Formula X shown below and possessing, by way of example and not limitation, the structures disclosed in Table 10. Furthermore, thiophene sulfonamide carbonic anhydrase inhibitors useful in the practice of the present methods are described in U.S. Pat. No. 5,646,142, which is herein incorporated by reference in its entirety.

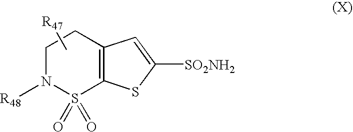

wherein:

R₄₇ is H; OH; C₁₋₆ alkoxy; C₁₋₆ alkyl unsubstituted or substituted optionally with OH, NR₄₉R₅₀, OC(═O)R₅₁ or C(═O)R₅₁; NR₄₉R₅₀; OC(═O)R₅₁; C(═O)R₅₁; C₂₋₄ alkoxy substituted optionally with OH, NR₄₉R₅₀, halogen, C₁₋₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘ R₅₃ or SO₂ NR₄₉R₅₀; wherein m is 0-2 and n is 0-2; provided that when R₄₇ is OH, alkoxy, NR₄₉R₅₀ or OC(═O)R₅₁ it is attached to the 4-position and when R₄₇ is R₅₂ and is attached to the 3 position, the R₅₂ ring is attached by a carbon carbon single bond;

R₄₈ is C₂₋₈ alkyl substituted with S(═O)ₘR₅₃; C₄₋₇ alkenyl substituted with S(═O)ₘR₅₃ wherein m is 0-2;

R₄₉ & R₅₀ are H; C₁₋₈ alkyl; C₂₋₄ alkyl substituted optionally with OH, halogen, C₁₋₄ alkoxy or C(═O)R₅₁; C₁₋₄ alkoxy; C₂₋₄ alkoxy substituted optionally with OH, halogen, C₁₋₄ alkoxy or C(═O)R₅₁; or R₄₉ and R₅₀ can be joined to form a ring of 5 or 6 atoms selected from O, S, C or N which can be unsubstituted or substituted optionally on carbon with OH, (═O), halogen, C₁₋₄ alkoxy, C(═O)R₅₁, C₁₋₆ alkyl, C₁₋₆ alkyl substituted optionally with OH, halogen, C₁₋₄ alkoxy, C(═O)R₅₁ or on nitrogen with C₁₋₄ alkoxy, C(═O)R₅₁, S(═O)ₘR₅₃, C₁₋₆ alkyl or C₂₋₆ alkyl substituted optionally with OH, halogen, C₁₋₄ alkoxy, C(═O)R₅₁ or on sulfur by (═O)ₘ, wherein m is 0-2;

R₅₁ is C₁₋₈ alkyl; C₁₋₈ alkyl substituted optionally with OH, NR₄₉R₅₀, halogen, C₁₋₄ alkoxy or C(═O)R₅₄; C₁₋₄ alkoxy; C₂₋₄ alkoxy substituted optionally with OH, NR4₉R₅₀, halogen or C₁₋₄ alkoxy; or NR₄₉R₅₀;

R₅₂ is a monocyclic ring system of 5 or 6 atoms composed of C, N, O or S, such as furan, thiophene, pyrrole, pyrazole, imidazole, triazole, tetrazole, oxazole, isoxazole, isothiazole, thiazole, thiadiazole, pyridine pyrimidine, pyridazine, and pyrazine;

R₅₃ is C₁₋₄ alkyl; C₃₋₅ alkenyl, C₂₋₄ alkyl substituted optionally with OH, NR₄₉R₅₀, C₁₋₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘC₁₋₄ alkyl or SO₂NR₄₉R₅₀;

m is 0-2 and n is 0-2; and

R₅₄ is C₁₋₄ alkyl; C₁₋₄ alkoxy; amino, C₁₋₃ alkylamino, or di-C₁₋₃ alkylamino.

TABLE 10

| Com- | |
|---|---|
| pound No. | Compound |
| A-138 | 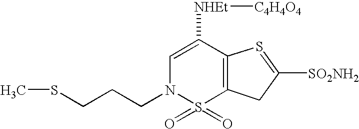 |
| | (+)-(R)-4-ethylamino-3,4-dihydro-2-(3-methylthiopropyl)-2H- |
| | thieno-[3,2-e]-1,2-thiazine-6-sulfonamide 1,1-dioxide maleic |
| | acid |
| A-139 | 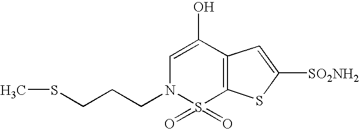 |
| | 3,4-dihydro-4-hydroxy-2-(3-methylthiopropyl)-2H-thieno- |
| | [3,2-e]-1,2-thiazine-6-sulfonamide 1,1-dioxide |
| A-140 | 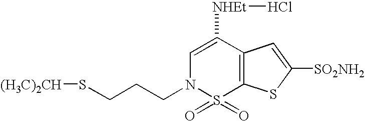 |
| | (R)-4-ethylamino-3,4-dihydro-2-[3-(1-methylethyl- |
| | thio)propyl]-2H-thieno-[3,2-e]-1,2-thiazine-6-sulfonamide |
| | 1,1-dioxide hydrochloride |
| A-141 | 3,4-dihydro-4-propylamino-2-(3-methylthiopropyl)-2H-thieno- |
| | [3,2-e]-1,2-thiazine-6-sulfonamide 1,1-dioxide |
| A-142 | 3,4-dihydro-4-[(2-methylpropyl)amino]-2-(3-methylthio- |
| | propyl)-2H-thieno-[3,2-e]-1,2-thiazine-6-sulfonamide 1,1- |
| | dioxide |
| A-143 | 3,4-dihydro-4-[(3-methylbutyl)amino]-2-(3-methylthiopropyl)- |
| | 2H-thieno-[3,2-e]-1,2-thiazine-6-sulfonamide 1,1-dioxide |
| A-144 | 3,4-Dihydro-4-methylamino-2-(3-methylthiopropyl)-2H- |
| | thieno-[3,2-e]-1,2-thia zine-6-sulfonamide 1,1-dioxide |
| A-145 | 3,4-Dihydro-4-hydroxy-2-(3-methylthiopropyl)-2H-thieno- |
| | [3,2-e]-1,2-thiazine-6-sulfonamide 1,1-dioxide |
| A-146 | 4-Ethylamino-3,4-dihydro-2-(3-propylthiopropyl)-2H-thieno- |
| | [3,2-e]-1,2-thiaz ine-6-sulfonamide 1,1-dioxide |
| A-147 | 4-Ethylamino-3,4-dihydro-2-(3-ethylthiopropyl)-2H-thieno- |
| | [3,2-e]-1,2-thiazi ne-6-sulfonamide 1,1-dioxide |
| A-148 | 4-Ethylamino-3,4-dihydro-2-[3-(2-methoxyethylthio)propyl]- |
| | 2H-thieno-[3,2-e]-1,2-thiazine-6-sulfonamide 1,1-dioxide |
| A-149 | 4-Ethylamino-3,4-dihydro-2-[3-(3-methoxypropylthio)propyl]- |
| | 2H-thieno-[3,2-e]-1,2-thiazine-6-sulfonamide 1,1-dioxide |
| A-150 | 4-Ethylamino-3,4-dihydro-2-[3-(2-methoxyethylthio)ethyl]- |
| | 2H-thieno-[3,2-e]-1,2-thiazine-6-sulfonamide 1,1-dioxide |
| A-151 | 4-Ethylamino-3,4-dihydro-2-[(2-methylthio)ethyl]-2H-thieno- |
| | [3,2-e]-1,2-thia zine-6-sulfonamide 1,1-dioxide |
| A-152 | 3,4-Dihydro-4-propylamino-2-[(2-methylthio)ethyl)]-2H- |
| | thieno-[3,2-e]-1,2-thiazine-6-sulfonamide 1,1-dioxide |
| A-153 | 3,4-Dihydro-4-[(2-methylpropyl)amino]-2-[(2-methylthio)- |
| | ethyl]-2H-thieno-[3,2-e]-1,2-thiazine-6-sulfonamide 1,1- |
| | dioxide |
| A-154 | 4-Ethylamino-3,4-dihydro-2-[(4-methylthio)butyl]-2H-thieno- |
| | [3,2-e]-1,2-thia zine-6-sulfonamide 1,1-dioxide |
| A-155 | 3,4-Dihydro-4-propylamino-2-[(4-methylthio)butyl]-2H- |
| | thieno-[3,2-e]-1,2-thi azine-6-sulfonamide 1,1-dioxide |
| A-156 | 3,4-Dihydro-4-[(2-methylpropyl)amino]-2-[(4-methylthio)- |
| | butyl]-2H-thieno-[3,2-e]-1,2-thiazine-6-sulfonamide 1,1- |
| | dioxide |

In yet another embodiment, the carbonic anhydrase inhibitor is selected from the class of sulfonamide carbonic anhydrase inhibitors represented by the general structure of Formula XI shown below and possessing, by way of example and not limitation, the structures disclosed in Table 11. Furthermore, sulfonamide carbonic anhydrase inhibitors useful in the practice of the present methods are described in U.S. Pat. Nos. 5,932,572 and 5,679,670, both of which are herein incorporated by reference in their entirety.

TABLE A

| | |
|---|---|
| | (XI) |
| 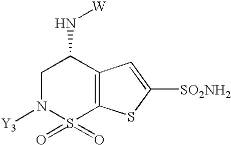 | |

wherein: W and Y₃ are as listed in Table A.

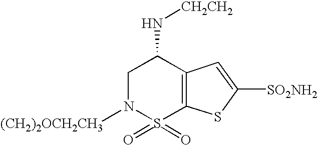

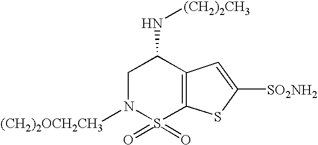

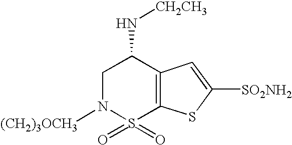

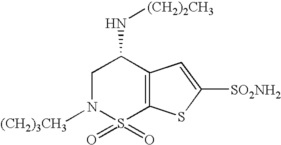

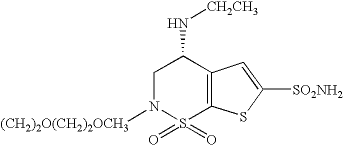

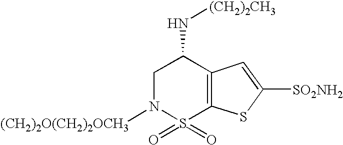

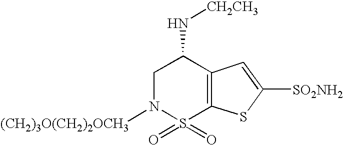

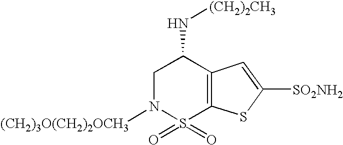

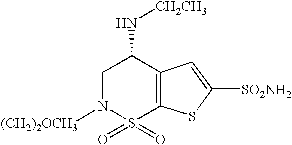

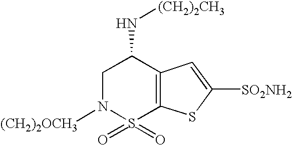

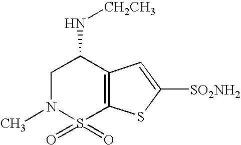

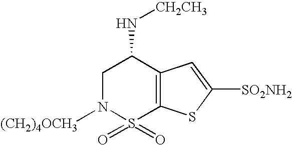

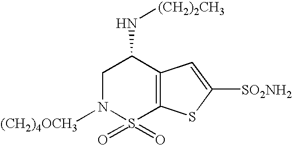

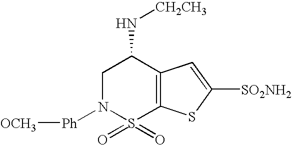

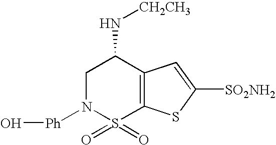

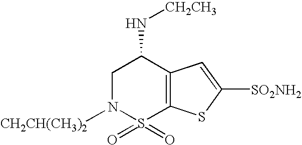

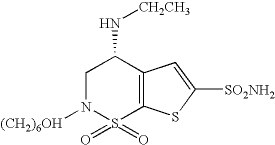

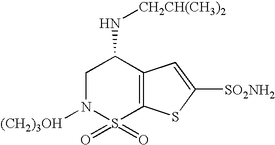

TABLE 11

| Compound | |
|---|---|
| No. | Compound |
| A-157 | 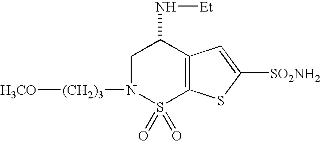 |
| | (+)-4-Ethylamino-3,4-dihydro-2-(3-methoxy)propyl-2H- |
| | thieno[3,2-e]-1,2-thiazine-6-sulfonamide-1,1-dioxide |
| | hydrochloride |
| A-158 | 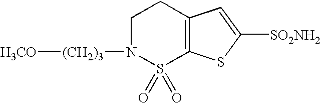 |
| | 3,4-Dihydro-2-(3-methoxypropyl)-2H-thieno[3,2-e]-1,2- |
| | thiazine-6-sulfonamide-1,1-dioxide sodium salt |
| A-159 | 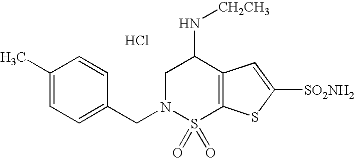 |
| | 4-Ethylamino-3,4-dihydro-2-(4-methylphenyl)methyl-2H- |
| | thieno[3,2-e]-1,2-thiazine-6-sulfonamide 1,1-dioxide |
| | hydrochloride |
| A-160 | 3,4-Dihydro-2-(3-phenylpropyl)-4-propylamino-2H- |
| | thieno[3,2-e]-1,2-thiazine-6-sulfonamide 1,1-dioxide |
| | hydrochloride |
| A-161 | 3,4-dihydro-2-(4-phenylbutyl)-4-propylamino-2H- |
| | thieno[3,2-e]-1,2-thiazine-6-sulfonamide 1,1-dioxide |
| | hydrochloride |
| A-162 | 4-Ethylamino-3,4-dihydro-2-(2-thienyl)methyl-2H- |
| | thieno[3,2-e]-1,2-thiazine-6-sulfonamide 1,1-dioxide |
| | hydrochloride |
| A-163 | 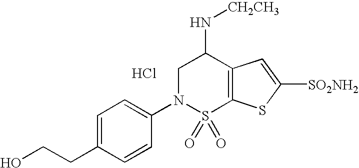 |
| | 4-Ethylamino-3,4-dihydro-2-[4-(2-hydroxyethyl)phenyl]- |
| | 2H-thieno[3,2-e]-1,2-thiazine-6-sulfonamide 1,1-dioxide |
| | hydrochloride |
| A-164 | 2-(4-n-Butylphenyl)-3,4-dihydro-4-propylamino-2H- |
| | thieno[3,2-e]-1,2-thiazine-6-sulfonamide 1,1-dioxide |
| | hydrochloride |
| A-165 | 3,4-Dihydro-2-phenyl-4-propylamino-2H-thieno[3,2-e]-1,2- |
| | thiazine-6-sulfonamide 1,1-dioxide tartrate |
| A-166 | (R)-4-Ethylamino-2-[4-(2-hydroxyethyl)phenyl]-3,4- |
| | dihydro-2H-thieno[3,2-e]-1,2-thiazine-6-sulfonamide 1,1- |
| | dioxide hydrochloride |
| A-167 | (R)-4-Ethylamino-2-(4-methoxy-phenyl)-3,4-dihydro-2H- |
| | thieno[3,2-e]-1,2-thiazine-6-sulfonamide 1,1-dioxide |
| | hydrochloride |
| A-168 | (R)-4-Ethylamino-2-(4-hydroxy-phenyl)-3,4-dihydro-2H- |
| | thieno[3,2-e]-1,2-thiazine-6-sulfonamide 1,1-dioxide |
| | hydrochloride |
| A-169 | (R)-3,4-Dihydro-2-(4-methoxy-phenyl)-4-propylamino-2H- |
| | thieno[3,2-e]-1,2-thiazine-6-sulfonamide 1,1-dioxide |
| | hydrochloride |
| A-170 | (R)-3,4-Dihydro-2-(4-hydroxy-phenyl)-4-propylamino-2H- |
| | thieno[3,2-e]-1,2-thiazine-6-sulfonamide 1,1-dioxide |
| | hydrochloride |
| A-171 | (R)-4-Ethylamino-3,4-dihydro-2-(3-methoxy-phenyl)-2H- |
| | thieno[3,2-e]-1,2-thiazine-6-sulfonamide 1,1-dioxide |
| | hydrochloride |
| A-172 | (R)-4-Ethylaminio-3,4-dihydro-2-(3-hydroxy-phenyl)-2H- |
| | thieno[3,2-e]-1,2-thiazine-6-sulfonamide 1,1-dioxide |
| | hydrochloride |
| A-173 | (R)-3,4-Dihydro-2-(3-methoxy-phenyl)-4-propylamino-2H- |
| | thieno[3,2-e]-1,2-thiazine-6-sulfonamide 1,1-dioxide |
| | hydrochloride |
| A-174 | (R)-3,4-Dihydro-2-(3-hydroxy-phenyl)-4-propylamino-2H- |
| | thieno[3,2-e]-1,2-thiazine-6-sulfonamide 1,1-dioxide |
| | hydrochloride |
| A-175 | 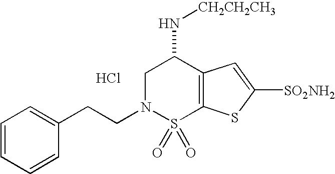 |
| | R-(+)-3,4-Dihydro-2-(2-phenylethyl)-4-propylamino-2H- |
| | thieno[3,2-e]-1,2-thiazine-6-sulfonamide 1,1-dioxide |
| | hydrochloride |
| A-176 | (R)-4-Ethylamino-3,4-dihydro-2-(4-methoxy- |
| | phenylmethyl)-2H-thieno[3,2-e]-1,2-thiazine-6-sulfonamide |
| | 1,1-dioxide hydrochloride |
| A-177 | (R)-4-Ethylamino-3,4-dihydro-2-(4-hydroxy-phenylmethyl)- |
| | 2H-thieno[3,2-e]-1,2-thiazine-6-sulfonamide 1,1-dioxide |
| | hydrochloride |
| A-178 | (R)-4-Ethylamino-3,4-dihydro-2-(3-methoxy- |
| | phenylmethyl)-2H-thieno[3,2-e]-1,2-thiazine-6-sulfonamide |
| | 1,1-dioxide hydrochloride |
| A-179 | (R)-4-Ethylamino-3,4-dihydro-2-(3-hydroxy-phenylmethyl)- |
| | 2H-thieno[3,2-e]-1,2-thiazine-6-sulfonamide 1,1-dioxide |
| | hydrochloride |
| A-180 | 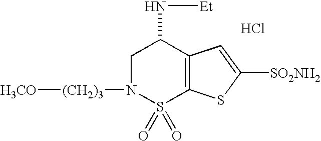 |
| | 3,4-Dihydro-2-(3-methoxypropyl)-3-methyl-2H-thieno>3,2- |
| | e!-1,2-thiazine-6-sulfonamide 1,1-dioxide |
| A-181 | 3,4-Dihydro-2,3-dimethyl-2H-thieno[3,2-e]-1,2-thiazine-6- |
| | sulfonamide 1,1-dioxide |
| A-182 | 3,4-Dihydro-2-(2-methoxyethyl)-3-methyl-2H-thieno[3,2- |
| | e]-1,2-thiazine-6-sulfonamide 1,1-dioxide |
| A-183 | 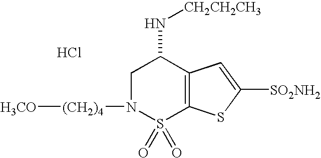 |
| | R-(+)-3,4-Dihydro-2-(4-methoxybutyl)-4-propylamino-2H- |
| | thieno[3,2-e]-1,2-thiazine-6-sulfonamide 1,1-dioxide |
| | hydrochloride |
| A-184 | 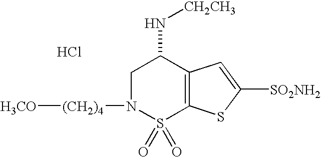 |
| | R-(+)-4-Ethylamino-3,4-dihydro-2-(4-methoxybutyl)-2H- |
| | thieno[3,2-e]-1,2-thiazine-6-sulfonamide 1,1-dioxide |
| | hydrochloride |
| A-185 | R-(+)-4-Ethylamino-3,4-dihydro-2-(6-hydroxyhexyl)-2H- |
| | thieno[3,2-e]-1,2-thiazine-6-sulfonamide 1,1-dioxide |
| | hydrochloride |
| A-186 | R-(+)-4-Allylamino-3,4-dihydro-2-(2-methylpropyl)-2H- |
| | thieno[3,2-e]-1,2-thiazine-6-sulfonamide 1,1-dioxide |
| | hydrochloride |
| A-187 | R-(+)-3,4-Dihydro-2-(4-hydroxybutyl)-4-propylamino-2H- |
| | thieno[3,2-e]-1,2-thiazine-6-sulfonamide 1,1-dioxide |
| | hydrochloride |
| A-188 | R-(+)-3,4-Dihydro-2-(2-methylpropyl)-4-propylamino-2H- |
| | thieno[3,2-e]-1,2-thiazine-6-sulfonamide 1,1-dioxide |
| | hydrochloride |
| A-189 | R-(+)-4-Ethylamino-3,4-dihydro-2-(2-methylpropyl)-2H- |
| | thieno[3,2-e]-1,2-thiazine-6-sulfonamide 1,1-dioxide |
| | hydrochloride |
| A-190 | R-(+)-4-Cyclopropylmethylamino-3,4-dihydro-2-(2- |
| | methylpropyl)-2H-thieno[3,2-e]-1,2-thiazine-6-sulfonamide |
| | 1,1-dioxide hydrochloride |
| A-191 | R-(+)-4-Ethylamino-3,4-dihydro-2-(3-methoxybutyl)-2H- |
| | thieno[3,2-e]-1,2-thiazine-6-sulfonamide 1,1-dioxide |
| | hydrochloride |
| A-192 | R-(+)-3,4-Dihydro-2-(3-methoxypropyl)-4-(2- |
| | methoxyethyl)amino-2H-thieno[3,2-e]-1,2-thiazine-6- |
| | sulfonamide 1,1-dioxide hydrochloride |
| A-193 | R-(+)-3,4-Dihydro-2-(3-methoxybutyl)-4-n-propylamino- |
| | 2H-thieno[3,2-e]-1,2-thiazine-6-sulfonamide 1,1-dioxide |
| | hydrochloride |
| A-194 | R-(+)-4-Ethylamino-3,4-dihydro-2-(4-hydroxybutyl)-2H- |
| | thieno[3,2-e]-1,2-thiazine-6-sulfonamide 1,1-dioxide |
| | hydrochloride |
| A-195 | (R)-3,4-Dihydro-2-(3-hydroxypropyl)-4-(2- |
| | methylpropyl)amino-2H-thieno[3,2-e]-1,2-thiazine-6- |
| | sulfonamide 1,1-dioxide hydrochloride |
| A-196 | 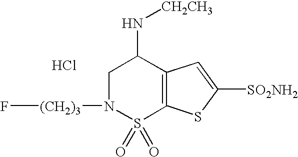 |
| | 4-Ethylamino-2-(3-fluoropropyl)-3,4-dihydro-2H- |
| | thieno[3,2-e]-1,2-thiazine-6-sulfonamide 1,1-dioxide |
| | hydrochloride |
| A-197 | 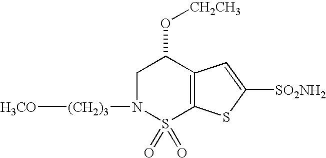 |
| | R-(−)-4-Ethoxy-3,4-dihydro-2-(3-methoxypropyl)-2H- |
| | thieno[3,2-e]-1,2-thiazine-6-sulfonamide 1,1-dioxide |
| | hydrochloride |
| A-198 | 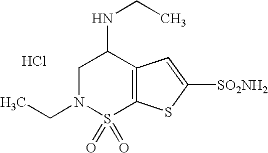 |
| | 6-Ethyl-4-ethylamino-4,5,6,7-tetrahydro-7-oxo-thieno[2,3- |
| | b]pyridine-2-sulfonamide hydrochloride |
| A-199 | 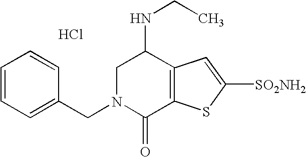 |
| | 4-Ethylamino-4,5,6,7-tetrahydro-7-oxo-6-(phenylmethyl)- |
| | thieno[2,3-b]pyridine-2-sulfonamide hydrochloride |
| A-200 | 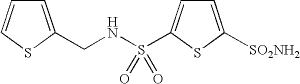 |
| | N-(2-Thienyl)methyl-2,5-thiophenedisulfonamide |
| A-201 | N-(4-Trifluoromethylphenyl)methyl-2,5- |
| | thiophenedisulfonamide |
| A-202 | N-(3,5-Dichlorophenyl)methyl-2,5-thiophenedisulfonamide |
| A-203 | N-(3,4-Dichlorophenyl)methyl-2,5-thiophenedisulfonamide |
| A-204 | N-(4-Methoxyphenyl)methyl-2,5-thiophenedisulfonamide |
| A-205 | N-(4-Fluorophenyl)methyl-2,5-thiophenedisulfonamide |
| A-206 | N-[[4-(4-Morpholinyl methyl)phenyl]methyl]-2,5-thiophene |
| | disulfonamide |
| A-207 | N-[[3-(4-Morpholinylmethyl)phenyl]methyl]-2,5- |
| | thiophenedisulfonamide hydrochloride |
| A-208 | 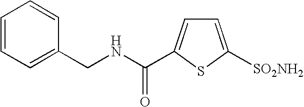 |
| | N-(Phenylmethyl)-5-(aminosulfonyl)-thiophene-2- |
| | carboxamide |

In a further embodiment, the carbonic anhydrase inhibitor is selected from the class of sulfonamide carbonic anhydrase inhibitors represented by the general structure of Formula XII shown below and possessing, by way of example and not limitation, the structures disclosed in Table 12. Furthermore, sulfonamide carbonic anhydrase inhibitors useful in the practice of the present methods are described in U.S. Pat. Nos. 6,248,735, 6,264,935 and 6,316,443, all of which are herein incorporated by reference in their entirety.

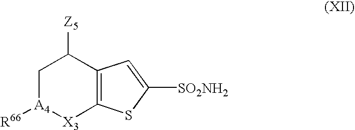

wherein:

A₄ is carbon or nitrogen;

Z₅ is NHR₆₅ or OR⁶⁵;

R⁶⁵ is C₁₋₆ alkyl, either straight or branched chain;

R⁶⁶ is hydrogen, C₁₋₃alkyl, or C₁₋₄ alkoxy-C-₁₋₄ alkyl; and

X₃ is S(O)₂ or C(O)₂.

TABLE 12

| Compound No. | Compound |
|---|---|
| A-209 | (S,S)-(−)-5,6-Dihydro-4-ethylamino-6-methyl-4H- |
| | thieno[2,3-b]thiopyran-2-sulfonamide-7,7-dioxide |
| | monohydrochloride |
| A-210 | (S,S)-(−)-5,6-Dihydro-4-ethylamino-6-(n-propyl)- |
| | 4H-thieno[2,3-b]thiopyran-2-sulfonamide-7,7- |
| | dioxide monohydrochloride |
| A-211 | (+−)-5,6-dihydro-4-[(2-methylpropyl)amino]- |
| | 4H-thieno[2,3-b]thiopyran-2-sulfonamide-7,7- |
| | dioxide monohydrochloride |
| A-212 | (S,S)-(−)-5,6-dihydro-4-ethylamino-6-methyl-4H- |
| | thieno[2,3-b]thiopyran-2-sulfonamide-7,7-dioxide |
| | monohydrochloride |
| A-213 | (S,S)-(−)-5,6-dihydro-4-ethylamino-6-methyl-4H- |
| | thieno[2,3-b]thiopyran-2-sulfonamide-7,7-dioxide |
| | monohydrochloride |
| A-214 | (S,S)-(−)-5,6-dihydro-4-ethylamino-6-methyl-4H- |
| | thieno[2,3-b]thiopyran-2-sulfonamide-7,7-dioxide |
| | monohydrochloride |

In another embodiment, the carbonic anhydrase inhibitor is selected from the class of sulfonamide carbonic anhydrase inhibitors represented by the general structure of Formulas XIIIa, XIIIb, XIIIc, and XTIId shown below and possessing, by way of example and not limitation, the structures disclosed in Table 13. Furthermore, sulfonamide carbonic anhydrase inhibitors useful in the practice of the present methods are described in U.S. Pat. No. 6,313,155, which is herein incorporated by reference in its entirety.

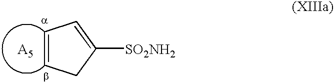

wherein A₅ together with the two carbon atoms denoted as α and β is the group:

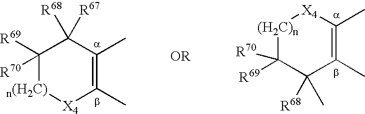

wherein:

X₄ is S, SO, SO₂ or CH₂;

Y₄ is S, O, or NR³ wherein R³ is hydrogen, C₁₋₃ alkyl, or benzyl;

n is 1 or 2;

R⁶⁷, R⁶⁸, R⁶⁹, R⁷⁰ are independently selected from:

(1) hydrogen,

(2) OR⁷¹ wherein R⁷¹ is:

(a) hydrogen,

(b) C₁₋₅ alkyl, either unsubstituted or substituted with OH, or wherein R⁷² and R⁷³ are independently hydrogen or C₁₋₅ alkyl, or joined together form a heterocycle with the nitrogen to which they are attached such as piperidino, morpholino, or piperazino,

(c) C₁₋₅ alkanoyl, either unsubstituted or substituted with OH, NR⁷²R⁷³, NHCOR⁷⁴ or COR⁷⁴ wherein R⁷⁴ is OH, NR⁷²R⁷³ or C₁₋₅ alkoxy,

(d) COR⁷⁵, wherein R⁷⁵ is NR⁷²R⁷³ or a 5 or 6-membered aromatic heterocycle such as pyridyl, imidazolyl, pyrazinyl, thiazolyl, thienyl, or oxazolyl,

(3) NR₇₃,

(4) NHR⁷⁶ wherein R⁷⁶ is:

(a) SO₂ NR⁷²R⁷³,

(b) SO₂R⁷⁷, wherein R⁷⁷ is C₁₋₅ alkyl, or

(c) CONR⁷²R⁷³,

(5) C₁₋₅ alkyl, either unsubstituted or substituted with

(a) OR⁷¹,

(b) CN,

(c) NR⁷²R⁷³, or

(d) COR⁷⁴,

(6) SO₂R⁷⁷,

(7) SO₂NR⁷²R⁷³, or

(8) halo, such as chloro, bromo or fluoro;

(9) R⁶⁷ and R⁶⁹, or R⁶⁸ and R⁷⁰ taken together represent a double bond;

(10) R⁶⁷ and R⁶⁸, or R⁶⁹ and R⁷⁰ taken together represent

(a) ═O, or

(b) ═NOR⁷⁸, wherein R⁷⁸ is hydrogen or C₁₋₃ alkyl; and

one of the CH₂ groups of (CH₂)ₙ can be substituted with COR⁷⁴, CH₂R⁷⁴, or CH₂ COR⁷⁴.

In yet another embodiment, the carbonic anhydrase inhibitor is selected from the class of sulfonamide carbonic anhydrase inhibitors represented by the general structure of Formula XIIIb.

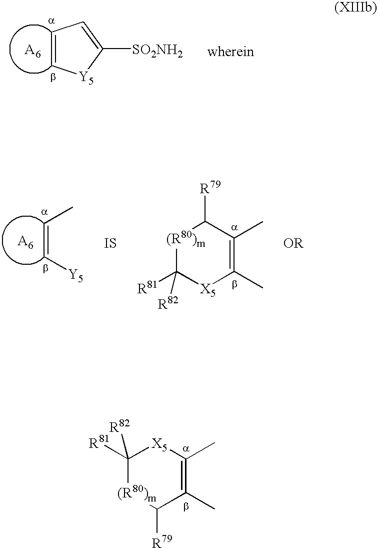

wherein:

X₅ is S, SO₂, or CH₂;

Y₅ is S, O, or NR⁸⁵, wherein R⁸⁵ is H, C₁₋₃ alkyl or benzyl,

m is 0 or 1,

R⁷⁹ is

(1) hydrogen,

(2) phenyl either unsubstituted or substituted with one or more of

(a) hydroxy,

(b) C₁₋₃ alkoxy,

(c) R⁸³R⁸⁴NC₁₋₅ alkyl wherein R⁸³ and R⁸⁴ are independently selected from:

(i) hydrogen and

(ii) C₁₋₅ alkyl, or taken together with the nitrogen to which they are attached form a heterocycle such as morpholine, piperidine, pyrrolidine, or piperazine,

(3) OH,

(4) ═O; or

(5) NR⁸³R⁸⁴,

R⁸⁰ is

(1) hydrogen,

(2) CN,

(3) phenyl-C₁₋₃ alkyl, wherein the phenyl is either unsubstituted or substituted with one or more of

(a) hydroxy,

(b) C₁₋₃ alkoxy, or

(c) R⁸³R⁸⁴NC₁₋₅ alkyl;

R⁸¹ is

(1) hydrogen,

(2) C₁₋₅ alkyl,

(3) phenyl-C₁₋₃ alkyl, wherein the phenyl is either unsubstituted or substituted with one or more of:

(a) hydroxy,

(b) C₁₋₃ alkoxy, or

(c) R⁸³R⁸⁴NC₁₋₃alkyl;

(4) phenyl either unsubstituted or substituted with one or more of:

(a) hydroxy,

(b) C₁₋₃ alkoxy, or

(c) R⁸³R⁸⁴NC₁₋₃ alkyl, or

(d) halo, such as chloro or fluoro

(5) aromatic heterocycle of 5 or 6 members such as furyl, pyridyl, or thienyl either unsubstituted or substituted with R⁸³R⁸⁴NC₁₋₃ alkyl,

(6) NR⁸³R⁸⁴, and

(7) C₂₋₅ alkyl substituted with NR⁸³R⁸⁴;

R⁸² is

(1) hydrogen,

(2) C₁₋₃ alkyl, or

(3) C₁₋₃ alkylene, such as methylene;

with the proviso that if R⁷⁹ is other than phenyl or substituted phenyl, and

R⁸⁰ is hydrogen, one of R⁸¹ and R⁸² is other than hydrogen.

In a further embodiment, the carbonic anhydrase inhibitor is selected from the class of sulfonamide carbonic anhydrase inhibitors represented by the general structure of Formula XIIIc.

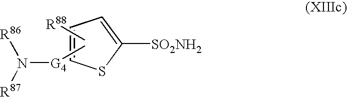

wherein:

R⁸⁶ is

(1) H,

(2) C₁₋₄ alkyl, or

(3) C₂₋₄ alkyl substituted with

(a) OH,

(b) halogen,

(c) C₁₋₄ alkoxy, or

(d) C(═O)R⁹²,

R⁸⁷ is

(1) H,

(2) C₁₋₈ alkyl,

(3) C₂₋₈ alkyl substituted with

(a) OH,

(b) NR⁹⁰R⁹¹,

(c) halogen

(d) C₁₋₄ alkoxy, or

(e) C(═O)R⁹²,

(4) C₃₋₇ alkenyl unsubstituted or substituted with

(a) OH,

(b) NR⁹⁰R⁹¹, or

(c) C₁₋₄ alkoxy,

(5) C₃₋₇ alkynyl, unsubstituted or substituted with

(a) OH,

(b) NR⁹¹R⁹¹, or

(c) C₁₋₄ alkoxy,

(6) C₁₋₃ alkyl substituted with

(a) phenyl, or

(b) heteroaryl, unsubstituted or substituted with

(i) OH,

(ii) (CH₂),NR⁹⁰R⁹¹,

(iii) halogen,

(iv) C₁₋₄ alkoxy,

(v) C₁₋₄ haloalkoxy,

(vi) C(═O)R⁹²,

(vii) S(═O)ₘR⁹³, or

(viii) SO₂NR⁹⁰R⁹¹;

wherein m is 0-2 and n is 0-2,

(7) C₂₋₄ alkoxy substituted with

(a) NR⁹⁰R⁹¹,

(b) halogen

(c) C₁₋₄ alkoxy, or

(d) C(═O)R⁹²;

(8) phenyl, or

(9) heteroaryl, unsubstituted or substituted with

(a) OH,

(b) (CH₂)ₙ NR⁹⁰R⁹¹,

(c) halogen,

(d) C₁₋₄ alkoxy,

(e) C₁₋₄ haloalkoxy,

(f) C(═O)R⁹²,

(g) S(═O)ₘR⁹³, or

(h) SO₂NR⁹⁰R⁹¹;

wherein m is 0-2 and n is 0-2,

with the proviso that R⁸⁶ and R⁸⁷ cannot both be H, or R⁸⁶ and R⁸⁷ can form a saturated ring of 5 or 6 atoms selected from O, S, C, or N, said ring being unsubstituted or substituted on C with

(1) OH,

(2) NR⁹⁰R⁹¹,

(3) halogen,

(4) C₁₋₄ alkoxy,

(5) C(═O)R⁹²,

(6) C₁₋₆ alkyl,

(7) C₁₋₆ alkyl substituted with

(a) OH,

(b) NR⁹⁰R⁹¹,

(c) halogen,

(d) C₁₋₄ alkoxy,

(e) C(═O)R⁹² or

substituted on N with

(1) NR⁹⁰R⁹¹,

(2) C₁₋₄ alkoxy,

(3) C(O)R⁹²

(4) C₁₋₆ alkyl,

(5) C₁₋₆ alkyl substituted with

(a) OH,

(b) NR⁹⁰R⁹¹,

(c) halogen,

(d) C₁₋₄ alkoxy, or

(e) C(═O)R⁹²;

R⁸⁸ is

(1) H,

(2) halogen,

(3) C₁₋₄ alkyl,

(4) C₁₋₈ alkoxy,

(5) C₁₋₈ alkylthiol,

(6) C₂₋₈ alkoxy substituted with

(a) OH,

(b) NR⁹⁰R⁹¹,

(c) halogen,

(d) C₁₋₄ alkoxy,

(e) C(═O)R⁹²,

(7) C₁₋₄ alkyl substituted with R⁸⁹,

(8) R⁸⁶ and R⁸⁷ form a ring of 5 to 7 members, said ring being unsubstituted or substituted with R⁸⁹;

R⁸⁹ is

(1) OH,

(2) C₁₋₄ alkyl unsubstituted or substituted with

(a) OH

(b) NR⁹⁰R⁹¹,

(c) halogen,

(d) C₁₋₄ alkoxy, or

(e) C(═O)R⁹².

(3) C₁₋₄ alkoxy,

(4) C₂₋₄ alkoxy substituted with

(a) OH,

(b) NR⁹⁰R⁹¹,

(c) halogen,

(d) C₁₋₄ alkoxy or

(e) C(═O)R⁹²,

(5) NR⁹⁰R⁹¹,

(6) phenyl, or

(7) heteroaryl, unsubstituted or substituted with

(a) OH,

(b) (CH₂)ₙNR⁹⁰R⁹¹,

(c) halogen,

(d) C₁₋₄ alkoxy,

(e) C₁₋₄ haloalkoxy,

(f) C(═O)R⁹²,

(g) S(═O)ₘR⁹³, or

(h) SO₂NR⁹⁰R⁹¹,

wherein m is 0-2 and n is 0-2;

with the proviso that when R⁸⁸ is in the 4 position and is H or halogen then R⁸⁶ and R⁸⁷ are not

(1) H,

(2) C₁₋₆ alkoxy substituted with

(a) OH,

(b) C₁₋₆ alkoxy,

(c) C₂₋₆ alkoxycarbonyl, or

(3) joined to form a 5, 6, or 7 member ring, saturated or unsaturated, comprised of atoms selected from C, O, S, N in which N, when saturated is substituted with H or C₁₋₆ alkyl or in which C is substituted with C₁₋₆ alkyl, C₁₋₆ alkoxy or OH;

and when R⁸⁸ is in the 5 position and is H, Cl, Br or C₁₋₃ alkyl then R⁸⁶ and R⁸⁷ are not H or C₁₋₄ alkyl;

R⁹⁰ and R⁹¹ are the same or different and are

(1) H,

(2) C₁₋₄ alkyl,

(3) C₂₋₄ alkyl substituted with

(a) OH,

(b) halogen,

(c) C₁₋₄ alkoxy, or

(d) C(═O)R⁹²,

(4) C₁₋₄ alkoxy,

(5) C₂₋₄ alkoxy substituted with

(a) OH,

(b) halogen,

(c) C₁₋₄ alkoxy, or

(d) C(═O)R⁹²,

(6) C₃₋₇ alkenyl unsubstituted or substituted with

(a) OH,

(b) NR⁹⁰R⁹¹, or

(c) C₁₋₄ alkoxy,

(7) C₃₋₇ alkynyl unsubstituted or substituted with

(a) OH,

(b) NR⁹⁰R⁹¹, or

(c) C₁₋₄ alkoxy,

(8) C₁₋₂ alkyl C₃₋₅ cycloalkyl or

(9) K⁹⁰ and R⁹¹ form a ring of 5 or 6 atoms selected from O, S, C, and N, said ring being unsubstituted or substituted on C with

(a) OH,

(b) (═O)

(c) halogen,

(d) C₁₋₄ alkoxy,

(e) C(═O)R⁹²,

(f) C₁₋₆ alkyl,

(g) C₁₋₆ alkyl substituted with

(i) OH,

(ii) halogen,

(iii) C₁₋₄ alkoxy,

(iv) C(═O)R⁹²,

or on N with

(a) C₁₋₄ alkoxy,

(b) C(═O)R⁹²,

(c) S(═O)ₘR⁹³,

(d) C₁₋₆ alkyl, or

(e) C₂₋₆ alkyl substituted with

(i) OH,

(ii) halogen,

(iii) C₁₋₄ alkoxy,

(iv) C(═O)R⁹²,

or on S with (═O)ₘ wherein m is 0-2;

R⁹² is

(1) C alkyl,

(2) C₁₋₈ alkyl substituted with

(a) OH,

(b) NR⁹⁰R⁹¹,

(c) halogen,

(d) C₁₋₄ alkoxy, or

(e) C(═O)R⁹⁴,

(3) C₁₋₄ alkoxy,

(4) C₂₋₄ alkoxy substituted with

(a) OH,

(b) NR⁹⁰R⁹¹,

(c) halogen, or

(d) C₁₋₄ alkoxy, or

(5) NR⁹⁰R⁹¹;

R⁹³ is

(1) C₁₋₄ alkyl,

(2) C₂₋₄ alkyl substituted with

(a) OH,

(b) NR⁹⁰R⁹¹,

(c) halogen,

(d) C₁₋₄ alkoxy, or

(e) C(═O)R⁹²;

R⁹⁴ is

(1) C₁₋₄ alkyl,

(2) C₁₋₄ alkoxy,

(3) amino,

(4) C₁₋₃ alkylamino or

(5) di-C₁₋₃ alkylamino, and

G₄ is C(═O) or SO₂.

In still further embodiment, the carbonic anhydrase inhibitor is selected from the class of sulfonamide carbonic anhydrase inhibitors represented by the general structure of Formula XIIId.

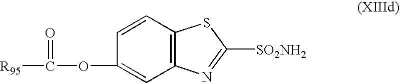

wherein R₉₅ is

(1) C₁₋₁₈ alkyl,

(2) C₃₋₆ cycloalkyl,

(3) C₃₋₆ cycloalkyl C₁₋₈ alkyl,

(4) C₁₋₁₈ alkyl C₃₋₆ cycloalkyl,

(5) haloalkyl,

(6) aryl, unsubstituted or substituted with

(a) C₁₋₁₀ alkyl, straight or branched,

(b) halo selected from bromo, chloro and fluoro, or

(c) alkoxy, selected from methoxy and ethoxy,

(7) arylalkyl, where alkyl is C₁₋₄ and aryl is unsubstituted or substituted with fluoro, chloro, bromo or C₁₋₃ alkyl,

(8) C₂₋₁₈ hydroxyalkyl,

(9) C₂₋₁₈ aminoalkyl,

(10) C₂₋₆ alkenyl,

(11) C₂₋₆ alkynyl, or

(12) aryl C₂₋₆ alkenyl.

TABLE 13

| Compound No. | Compound |
|---|---|
| A-215 | 5,6-dihydro-4-ethylamino-6-methyl-4H-thieno[2,3-b] |
| | thiopyran-2-sulfonamide-7,7-dioxide |
| A-216 | 5,6-dihydro-4-(2-methylpropylamino)-6-methyl-4H- |
| | thieno[2,3-b]thiopyran-2-sulfonamide-7,7-dioxide |
| A-217 | 5,6-dihydro-6,6-dimethyl-4-ethylamino-4H-thieno[2,3- |
| | b]thiopyran-2-sulfonamide-7,7-dioxide |
| A-218 | 5,6-dihydro-5-(3-dimethylaminomethyl-4- |
| | hydroxybenzyl)-4H-thieno[2,3-b]thiopyran-2- |
| | sulfonamide-7,7-dioxide |
| A-219 | 5,6-dihydro-6-(3-dimethylaminomethyl-4- |
| | hydroxyphenyl)-4H-thieno[2,3-b]thiopyran-2- |
| | sulfonamide-7,7-dioxide |
| A-220 | (+)-3,4-dihydro-4-ethylamino-2-methyl-4H- |
| | thieno[3,2-e]-1,2-thiazine-6-sulfonamide-1,1-dioxide |
| A-221 | 3,4-dihydro-4-methoxy-2-methyl-4H-thieno[3,2-e]-1,2- |
| | thiazine-6-sulfonamide-1,1-dioxide |
| A-222 | 3,4-dihydro-2-methyl-4(2-methyl)propylamino- |
| | 4H-thieno[3,2-e]-1,2-thiazine-6-sulfonamide-1,1-dioxide |
| A-223 | 3,4-dihydro-4-methoxy-2-[2-(4-morpholino)ethyl]-4H- |
| | thieno[3,2-e]-1,2-thiazine-6-sulfonamide-1,1-dioxide |
| A-224 | 3,4-dihydro-4-ethylamino-2-allyl-4H-thieno[3,2- |
| | e]-1,2-thiazine-6-sulfonamide-1,1-dioxide |
| A-225 | 3,4-dihydro-4-ethylamino-2-n-propyl-4H- |
| | thieno[3,2-e]-1,2-thiazine-6-sulfonamide-1,1-dioxide |
| A-226 | 3,4-dihydro-4-ethylamino-2-(2-methoxyethyl)-4H- |
| | thieno[3,2-e]-1,2-thiazine-6-sulfonamide-1,1-dioxide |
| A-226 | 3,4-dihydro-4-hydroxy-2-[2-(4-morpholino)ethyl]-4H- |
| | thieno[3,2-e]-1,2-thiazine-6-sulfonamide-1,1-dioxide |

The carbonic anhydrase inhibitor employed in the present invention can exist in tautomeric, geometric or stereoisomeric forms. Generally speaking, suitable carbonic anhydrase inhibitors that are in tautomeric, geometric or stereoisomeric forms are those compounds that inhibit carbonic anhydrase activity by about 25%, more typically by about 50%, and even more typically, by about 75% or more when present at a concentration of 100 μM or less. The present invention contemplates all such compounds, including cis- and trans-geometric isomers, E- and Z-geometric isomers, R- and S-enantiomers, diastereomers, d-isomers, 1-isomers, the racemic mixtures thereof and other mixtures thereof. Pharmaceutically acceptable salts of such tautomeric, geometric or stereoisomeric forms are also included within the invention. The terms “cis” and “trans”, as used herein, denote a form of geometric isomerism in which two carbon atoms connected by a double bond will each have a hydrogen atom on the same side of the double bond (“cis”) or on opposite sides of the double bond (“trans”). Some of the compounds described contain alkenyl groups, and are meant to include both cis and trans or “E” and “Z” geometric forms. Furthermore, some of the compounds described contain one or more stereocenters and are meant to include R, S, and mixtures or R and S forms for each stereocenter present.

Generally speaking, the pharmacokinetics of the particular agent to be administered will dictate the most preferred method of administration and dosing regiment. The carbonic anhydrase inhibitor can be administered as a pharmaceutical composition with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17.sup.th Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Moreover, the carbonic anhydrase inhibitor can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, or magnesium carbonate.

In another embodiment, the carbonic anhydrase inhibitor can be administered intravenously, parenterally, intramuscular, subcutaneously, orally, nasally, topically, by inhalation, by implant, by injection, or by suppository. For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used. Pulmonary administration can be accomplished, for example, using any of various delivery devices known in the art such as an inhaler. See. e.g. S. P. Newman (1984) in Aerosols and the Lung, Clarke and Davis (eds.), Butterworths, London, England, pp. 197-224; PCT Publication No. WO 92/16192; PCT Publication No. WO 91/08760. For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like.

The actual effective amounts of compound or drug can and will vary according to the specific composition being utilized, the mode of administration and the age, weight and condition of the subject. Dosages for a particular individual subject can be determined by one of ordinary skill in the art using conventional considerations. But in general, the amount of carbonic anhydrase inhibitor will be between about 0.5 to about 2000 milligrams per day and more typically, between about 100 to about 1000 milligrams per day. The daily dose can be administered in one to four doses per day.

By way of example, in one embodiment when the carbonic anhydrase inhibitor is acetazolamide administered orally, the daily dosage is typically from about 250 to about 1000 milligrams per day administered in one to four doses per day. In another embodiment, when the carbonic anhydrase inhibitor is acetazolamide administered as an injection, the daily dosage is typically from about 100 to about 500 milligrams per day, but it is administered in one or two doses per day.

By way of further example, in another embodiment when the carbonic anhydrase inhibitor is dichlorphenamide administered orally, the daily dosage is typically from about 25 to about 200 milligrams administered in one to three doses per day.

By way of yet further example, in another embodiment when the carbonic anhydrase inhibitor is methazolamide administered orally, the daily dosage is typically from about 75 to about 300 milligrams administered in one to three doses per day.

In general, the timing of the administration of the cyclooxygenase-2 selective inhibitor in relation to the administration of the carbonic anhydrase inhibitor may also vary from subject to subject. In one embodiment, the cyclooxygenase-2 selective inhibitor and carbonic anhydrase inhibitor may be administered substantially simultaneously, meaning that both agents may be administered to the subject at approximately the same time. For example, the cyclooxygenase-2 selective is administered during a continuous period beginning on the same day as the beginning of the carbonic anhydrase inhibitor and extending to a period after the end of the carbonic anhydrase inhibitor. Alternatively, the cyclooxygenase-2 selective inhibitor and carbonic anhydrase inhibitor may be administered sequentially, meaning that they are administered at separate times during separate treatments. In one embodiment, for example, the cyclooxygenase-2 selective inhibitor is administered during a continuous period beginning prior to administration of the carbonic anhydrase inhibitor and ending after administration of the carbonic anhydrase inhibitor. Of course, it is also possible that the cyclooxygenase-2 selective inhibitor may be administered either more or less frequently than the carbonic anhydrase inhibitor. Moreover, it will be apparent to those skilled in the art that it is possible, and perhaps desirable, to combine various times and methods of administration in the practice of the present invention.

Indication to be Treated

Generally speaking, the composition comprising a therapeutically effective amount of a cyclooxygenase-2 selective inhibitor and a therapeutically effective amount of a carbonic anhydrase inhibitor may be employed to treat any type of neoplasia or neoplasia related disorder in a subject irrespective of its stage of progression.

In some aspects, the composition may be administered to either prevent the onset of clinically evident neoplasia altogether or to prevent the onset of a preclinically evident stage of neoplasia in subjects at risk for developing neoplasia. In other aspects, the composition may be administered to prevent the initiation of malignant cells or to arrest or reverse the progression of premalignant cells to malignant cells. In still other aspects, the composition may be administered to inhibit neoplasia growth, spreading or metastasis, as well as partial or total destruction of the neoplasia cells.

The composition may be effectively employed to treat a number of different types of neoplasia. In one embodiment, the neoplasia is epithelial cell-derived neoplasia (epithelial carcinoma). By way of example, epithelial cell-derived neoplasia includes basal cell carcinoma, squamous cell carcinoma or adenocarcinoma. In another embodiment, the neoplasia is a gastrointestinal cancer. Gastrointestinal cancers include lip cancer, mouth cancer, esophogeal cancer, small bowel cancer, stomach cancer and colon cancer. In still another embodiment, the neoplasia is liver cancer, bladder cancer, pancreas cancer, ovary cancer, cervical cancer, lung cancer, breast cancer and skin cancer, such as squamous cell and basal cell cancers, prostate cancer, brain cancer and renal cell carcinoma. The composition can also be used to treat fibrosis that often occurs with radiation therapy. In yet another embodiment, the composition can be used to treat subjects having adenomatous polyps, including those with familial adenomatous polyposis (FAP).

The cyclooxygenase-2 selective inhibitor and carbonic anhydrase inhibitor may also be administered with any other drug or agent known in the art to have utility for treating or preventing neoplasia disorders or related diseases. In one embodiment, the antineoplastic agent is an antimetabolite including folate antagonists (e.g. methotrexate), pyrimidine antagonists (e.g. cytarabine, floxuridine, fludarabine, fluorouracil, and gemcitabine), purine antagonists (e.g. cladribine, mercaptopurine, thioguanine), and adenosine deaminase inhibitors (e.g. pentostatin). In an alternative embodiment, the antineoplastic agent is an alkylating agent such as chlorambucil, cyclophosphamide, busulfan, ifosfamide, melphalan, and thiotepa. In yet another embodiment, the antineoplastic agent is an akylator agent such as cisplatin, carboplatin, procarbazine, dacarbazine, and altretamine. In still another embodiment, the antineoplastic agent is an anti-tumor antibiotic such as bleomycin, dactinomycin, and mitomycin. In yet a further embodiment, the antineoplastic agent is an immunological agent such as interferon. In another embodiment, the antineoplastic agent is a plant alkaloid including vinca alkaloids (e.g. vinblastine vincristine and vinorelbine), epipodophyllotoxins (e.g. etoposide and teniposide), taxanes (e.g. docetaxel and paclitaxel), and camptothecins (e.g. topotecan and irinotecan). Of course those skilled in the art will appreciate that the particular antineoplastic agents to be administered with the composition of the invention will vary considerably depending on the type of neoplasia disorder being treated and its stage of progression.

### EXAMPLES

### Example 1

### Determining Whether A Composition Reduces Tumor Cell Growth

The ability of a composition of the invention to reduce the growth of tumor cells can readily be determined. As used in the examples, the term “composition” shall include any composition comprising a cyclooxygenase-2 selective inhibitor and carbonic anhydrase inhibitor detailed herein. By way of example, the cyclooxygenase-2 selective inhibitor utilized for testing the composition may be celecoxib, rofecoxib, valdecoxib, etoricoxib, parecoxib, or deracoxib. The carbonic anhydrase inhibitor may include acetazolamide, methazolamide, dorzolamide, or brinzolamide. Moreover, various cell lines can be used to determine whether the composition reduces growth of tumor cells. For example, these cell lines include: SW-480 (colonic adenocarcinoma); HT-29 (colonic adenocarcinoma), A-427 (lung adenocarcinoma carcinoma); MCF-7 (breast adenocarcinoma); UACC-375 (melanoma line); and DU-145 (prostrate carcinoma). Cytotoxicity data obtained using these cell lines are indicative of an inhibitory effect on neoplastic lesions. These cell lines are well characterized, and are used by the United States National Cancer Institute in their screening program for new anti-cancer drugs.

By way of illustration, a composition's ability to inhibit tumor cell growth can be measured using the HT-29 human colon carcinoma cell line obtained from ATCC and a SRB assay. HT-29 cells have previously been characterized as a relevant colon tumor cell culture model and may be (Fogh, J., and Trempe, G. In: Human Tumor Cells in Vitro, J. Fogh (eds.), Plenum Press, New York, pp. 115-159, 1975). In this assay, HT-29 cells are maintained in RPMI media supplemented with 5% fetal bovine calf serum (Gemini Bioproducts, Inc., Carlsbad, Calif.) and 2 mm glutamine, and 1% antibiotic-antimycotic in a humidified atmosphere of 95% air and 5% CO₂ at 37° C. Briefly, HT-29 cells are plated at a density of 500 cells/well in 96 well microtiter plates and incubated for 24 hours at 37° C. prior to the addition of compound. Each determination of cell number involves six replicates. After six days in culture, the cells are fixed by the addition of cold trichloroacetic acid to a final concentration of 10% and protein levels are measured using the sulforhodamine B (SRB) colorimetric protein stain assay as previously described by Skehan, P., Storeng, R., Scudiero, D., Monks, A., McMahon, J., Vistica, D., Warren, J. T., Bokesch, H., Kenney, S., and Boyd, M. R., “New Colorimetric Assay For Anticancer-Drug Screening,” J. Natl. Cancer Inst. 82: 1107-1112, 1990, which is incorporated herein by reference.

In addition to the SRB assay described above, a number of other methods are available to measure growth inhibition and could be substituted for the SRB assay. These methods include counting viable cells following trypan blue staining, labeling cells capable of DNA synthesis with BrdU or radiolabeled thymidine, neutral red staining of viable cells, or MTT staining of viable cells.

Significant tumor cell growth inhibition greater than about 50% at a therapeutically effective dose is indicative that the composition is useful for treating neoplastic lesions.

### Example 2

### Mammary Gland Organ Culture Model Tests

Compositions can also be tested for antineoplastic activity by their ability to inhibit the incidence of pre-neoplastic lesions in a mammary gland organ culture system. This mouse mammary gland organ culture technique has been successfully used by other investigators to study the effects of known antineoplastic agents such as certain NSAIDs, retinoids, tamoxifen, selenium, and certain natural products.

For example, female BALB/c mice can be treated with a combination of estradiol and progesterone daily, in order to prime the glands to be responsive to hormones in vitro. The animals are sacrificed, and thoracic mammary glands are excised aseptically and incubated for ten days in growth media supplemented with insulin, prolactin, hydrocortisone, and aldosterone. DMBA (7,12 dimethylbenz(a)anthracene) is added to medium to induce the formation of premalignant lesions. Fully developed glands are then deprived of prolactin, hydrocortisone, and aldosterone, resulting in the regression of the glands but not the pre-malignant lesions.

The test composition is dissolved in DMSO and added to the culture media for the duration of the culture period. At the end of the culture period, the glands are fixed in 10% formalin, stained with alum carmine, and mounted on glass slides. The incidence of forming mammary lesions is the ratio of the glands with mammary lesions to glands without lesions. The incidence of mammary lesions in test composition treated glands is compared with that of the untreated glands.

The extent of the area occupied by the mammary lesions can be quantitated by projecting an image of the gland onto a digitation pad. The area covered by the gland is traced on the pad and considered as 100% of the area. The space covered by each of the non-regressed structures is also outlined on the digitization pad and quantitated by the computer.

What is claimed is:

1. A method for the treatment of neoplasia in a subject, the method comprising administering to the subject a cyclooxygenase-2 selective inhibitor or pharmaceutically acceptable salt or prodrug thereof and a carbonic anhydrase inhibitor or pharmaceutically acceptable salt or prodrug thereof.

2. The method of claim 1 wherein the carbonic anhydrase inhibitor comprises a benzothiazole sulfonamide.

3. The method of claim 2 wherein the carbonic anhydrase inhibitor comprises a compound having the formula 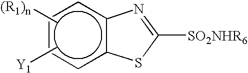
wherein:
each R₁ is hydrogen, lower alkyl, halogen, nitro, trihaloalkyl, lower alkoxy, formyl, lower alkanoyl loweralkylamino or diloweralkylamino;
R₆ is hydrogen or lower alkyl;
Y₁ is: 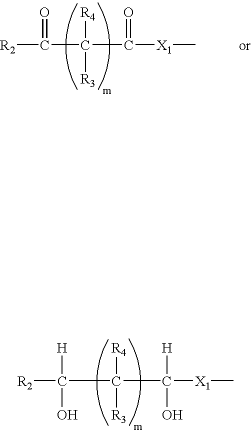
wherein:
X₁ is O or NR₅ or S;
R₂ is OR₇ or NR₇ R₈;
each R₃ and R₄ are hydrogen or lower alkyl;
R₅, R₇ and R are independently hydrogen or lower alkyl;
m is an integer which is 0, 1, 2, 3, 4, 5, or 6, and
n is an integer which is 0, 1, 2, or 3.

4. The method of claim 3 wherein the carbonic anhydrase inhibitor is selected from the group consisting of:
a) 6-hydroxy-2-benzothiazole sulfonamide;
b) 6-(ethyloxalyloxy)-2-benzothiazole sulfonamide;
c) 6-(ethylsuccinyloxy)-2-benzothiazole sulfonamide;

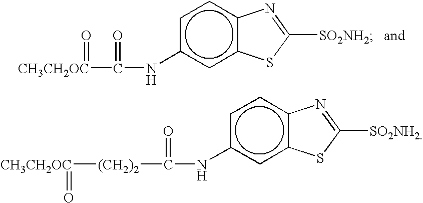

5. The method of claim 2 wherein the carbonic anhydrase inhibitor comprises a compound having the formula 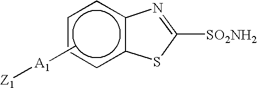
wherein:
Z₁ represents a water soluble carrier, and
A₁ is a moiety which is attached to the carbonic anhydrase inhibitor which allows it to still retain carbonic anhydrase inhibitory activity, but also form an enzymatically cleavable bond between A₁ and Z₁.

6. The method of claim 1 wherein the carbonic anhydrase inhibitor comprises a hydroxymethazolamide.

7. The method of claim 6 wherein the carbonic anhydrase inhibitor comprises a compound having the formula 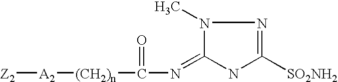
wherein:
Z₂ represents a water soluble carrier,
n is 1,2,3,4, or 5; and
A₂ is a moiety which is attached to the carbonic anhydrase inhibitor which allows it to still retain carbonic anhydrase inhibitory activity, but also form an enzymatically cleavable bond between A₂ and Z₂.

8. The method of claim 1 wherein the carbonic anhydrase inhibitor comprises a compound having the formula 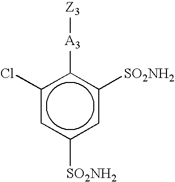
wherein:
Z₃ represents a water soluble carrier; and
A₃ is a moiety which is attached to the carbonic anhydrase inhibitor which allows it to still retain carbonic anhydrase inhibitory activity, but also form an, enzymatically cleavable bond between A₃ and Z₃.

9. The method of claim 1 wherein the carbonic anhydrase inhibitor comprises a compound having the formula 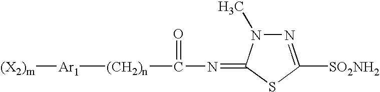
wherein:
n is an integer which is 0, 1, 2, 3,4, or 5;
X₂ is hydrogen, hydroxyl, hydroxylmethyl, 2-hydroxyethyl, or 2-hydroyethoxy;
Ar₁ is phenyl, pyridyl, or furanyl; and
m is an integer which is 0, 1, 2, 3, or 4.

10. The method of claim 1 wherein the carbonic anhydrase inhibitor comprises a thiophene sulfonamide.

11. The method of claim 10 wherein the carbonic anhydrase inhibitor comprises a compound having the formula 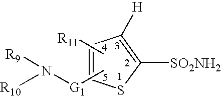
wherein:
R₉ is H, C₁₋₄ alkyl, C₂₋₄ alkyl substituted optionally with OH, halogen, C₁₋₄ alkoxy, or C(═O)R₁₅;
R₁₀ is H; C₁₋₈ alkyl; C₂₋₈ alkyl substituted with OH, NR₁₃R₁₄, halogen, C₁₋₄ alkoxy or C(═O)R₁₅; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₃ alkyl substituted with phenyl or heteroaryl which can be unsubstituted or substituted optionally with OH, (CH₂)ₙNR₁₃R₁₄, halogen, C₁₋₄ alkoxy, C₁₋₄ haloalkoxy, C(═O)R₁₅, S(═O)ₘR₁₆ or SO₂NR₁₃R₁₄, wherein m is 0-2 and n is 0-2; C₂₋₄ alkoxy substituted optionally with NR₁₃R₁₄, halogen, C₁₋₄ alkoxy, or C(═O)R₁₅; phenyl, or heteroaryl, unsubstituted or substituted optionally with OH, (CH₂)ₙ NR₁₃R₁₄, halogen, C₁₋₄ alkoxy, C₁₋₄ haloalkoxy, C(═O)R₁₅, S(═O)ₘ R₁₆ or SO₂ NR₁₃R₁₄, wherein m is 0-2 and n is 0-2; provided that R₉ and R₁₀ cannot both be H; or R₉ and R₁₀ can be joined to form a saturated ring of 5 or 6 atoms selected from O, S, C or N which can be unsubstituted or substituted optionally on carbon with OH, NR₁₃R₁₄, halogen, C₁₋₄ alkoxy, C(═O)R₁₅, C₁₋₆ alkyl, C₁₋₆ alkyl substituted optionally with OH, NR₁₃R₁₄, halogen, C₁₋₄ alkoxy, C(═O)R₁₅ or on nitrogen with NR₁₃R₁₄, C₁₋₄ alkoxy, C(═O)R₁₅, C₁₋₆ alkyl or C₂₋₆ alkyl substituted optionally with OH, NR₁₃R₁₄, halogen, C₁₋₄ alkoxy or C(═O)R₁₅;
R₁₁ is H; halogen; C₁₋₄ alkyl; C₁₋₈ alkoxy; C₁₋₈ alkylthiol; C₂₋₈ alkoxy substituted optionally with OH, NR₁₃R₁₄, halogen, C₁₋₄ alkoxy or C(═O)R₁₅; C₁₋₄ alkyl substituted optionally with R₁₂; or R₉ and R₁₁ can be joined together with carbon atoms to form a ring of from 5 to 7 members in which said carbon atoms can be unsubstituted or substituted optionally with R₁₂;
R₁₂ is OH; C₁₋₄ alkyl unsubstituted or substituted optionally with OH, NR₁₃R₁₄, halogen, C₁₋₄ alkoxy or C(═O)R₁₅; C₁₋₄ alkoxy; C₂₋₄ alkoxy substituted optionally with OH, NR₁₃R₁₄, halogen, C₁₋₄ alkoxy or C(═O)R₁₅; NR₁₃R₁₄; phenyl, or heteroaryl, unsubstituted or substituted optionally with OH, (CH₂)ₙ NR₁₃R₁₄, halogen, C₁₋₄ alkoxy, C₁₋₄haloalkoxy, C(═O)R₁₅, S(═O)ₘ R₁₆ or SO₂NR₁₃R₁₄, wherein m is 0-2 and n is 0-2;
R₁₃ and R₁₄ are the same or different and are H; C₁₋₄ alkyl; C₂₋₄ alkyl substituted optionally with OH, halogen, C₁₋₄ alkoxy or C(═O)R₁₅; C₁₋₄ alkoxy; C₂₋₄ alkoxy substituted optionally with OH, halogen, C₁₋₄ alkoxy or C(═O)R]5; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₂ alkylC₃₋₅ cycloalkyl; or R₁₃ and R₁₄ can be joined to form a ring of 5 or 6 atoms selected from O, S, C or N which can be unsubstituted or substituted optionally on carbon with OH, (═O), halogen, C₁₋₄ alkoxy, C(═O)R₁₅, C₁₋₆ alkyl, C₁₋₆ alkyl substituted optionally with OH, halogen, C₁₋₄ alkoxy, C(═O)R₁₅ or on nitrogen with C₁₋₄ alkoxy, C(═O)R₁₅, S(═O)ₘR₁₆, C₁₋₆ alkyl or C₂₋₆ alkyl substituted optionally with OH, halogen, C₁₋₄ alkoxy, C(═O)R₁₅ or on sulfur by (═O)ₘ, wherein m is 0-2;
R₁₅ is C₁₋₈ alkyl; C₁₋₈ alkyl substituted optionally with OH, NR₁₃R₁₄, halogen, C₁₋₄ alkoxy or C(═O)R₁₇; C₁₋₄ alkoxy; C₂₋₄ alkoxy substituted optionally with OH, NR₁₃R₁₄, halogen or C₁₋₄ alkoxy; or NR₁₃R₁₄;
R₁₆ is C₁₋₄ alkyl; C₂₋₄ alkyl substituted optionally with OH, NR₁₃R₁₄, halogen, C₁₋₄ alkoxy or C(═O)R₁₅; and
R₁₇ is C₁₋₄ alkyl; C₁₋₄ alkoxy; amino, C₁₋₃ alkylamino, or di-C₁₋₃ alkylamino; and G₁ is C(═O) or SO₂.

12. The method of claim 1 wherein the carbonic anhydrase inhibitor comprises a thienothiazine sulfonamide.

13. The method of claim 12 wherein the carbonic anhydrase inhibitor or pharmaceutically acceptable salt or prodrug thereof comprises a compound having the formula 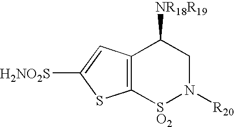
wherein:
R₁₈ and R₁₉ are H or C₁₋₄ alkyl;
R₂₀ is C₁₋₆ alkyl, CH₂(CH₂)ₙOR_{21,} where n is 1-4; and
R₂₁ is CH₃, (CH₂)ₙCH₃ where n is 1-4, or (CH₂)ₙAr₂ where Ar₂ is unsubstituted phenyl, 3-methoxyphenyl, or 4-methoxyphenyl and n is 1 or 2

14. The method of claim 12 wherein the carbonic anhydrase inhibitor comprises a compound having the formula 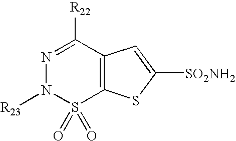
wherein:
R₂₂ is H, C₁₋₆ alkyl unsubstituted or substituted optionally with OH, C₁₋₄ alkoxy, NR₂₄R₂₅, OC(═O)R₂₆ or C(═O)R₂₆;
R₂₃ is H; C₁₋₈ alkyl; C₁₋₈ alkyl substituted with OH, NR₂₄R₂₅, halogen, C₁₋₄ alkoxy, C₂₋₄ alkoxy, C₁₋₄ alkoxy, OC(═O)R₂₆, S(═O)ₘ R₂₈, or C(═O)R₂₆; C₃₋₇ alkenyl unsubstituted or substituted optionally with OH, NR₂₄R₂₅, or C₁₋₄ alkoxy; C₃₋₇ alkynyl unsubstituted or substituted optionally with OH, NR₂₄R₂₅, or C₁₋₄ alkoxy; C₀₋₃ alkyl substituted with R₂₇ which can be unsubstituted or substituted optionally with C₁₋₃ alkyl, C₁₋₃ haloalkyl, OH, (CH₂), NR₂₄R₂₅, halogen, C₁₋₄ alkoxy, C₁₋₄ haloalkoxy, OC(═O)R₂₆, C(═O)R₂₆, S(═O)ₘ R₂₈ or SO₂ NR₂₄R₂₅, wherein m is 0-2 and n is 0-2;
R₂₄ and R₂₅ are independently H; C₁₋₈ alkyl; C₂₋₄ alkyl substituted optionally with OH, halogen, C₁₋₄ alkoxy or C(═O)R₂₆; OH; C₁₋₄ alkoxy; C₂₋₄ alkoxy substituted optionally with OH, halogen, C₁₋₄ alkoxy or C(═O)R₂₆; or R₂₄ and R₂₅ can be joined to form a ring of 5 or 6 atoms selected from O, S, C or N which can be unsubstituted or substituted optionally on carbon with OH, (═O), halogen, C₁₋₄ alkoxy, C(═O)R₂₆, C₁₋₆ alkyl, C₁₋₆ alkyl substituted optionally with OH, halogen, C₁₋₄ alkoxy, C(═O)R₂₆ or on nitrogen with C₁₋₄ alkoxy, C((═O)R₂₆, S(═O)ₘ R₂₈, C₁₋₆ alkyl or C₂₋₆ alkyl substituted optionally with OH, halogen, C₁₋₄ alkoxy, C(═O)R₂₆ or on sulfur by (═O)ₘ, where m is 0-2;
R₂₆ is C₁₋₈ alkyl; C₁₋₄ alkyl substituted optionally with OH, NR₂₄R₂₅, halogen, C₁₋₄ alkoxy or C(═O)R₂₉; C₁₋₄ alkoxy; C₂₋₄ alkoxy substituted optionally with OH, NR₂₄R₂₅, halogen or C₁₋₄ alkoxy; or NR₂₄R₂₅;
R₂₇, is a monocyclic ring system of 5 or 6 atoms composed of C, N, O or S, such as benzene, furan, thiophene, pyrrole, pyrazole, imidazole, triazole, tetrazole, oxazole, isoxazole, isothiazole, thiazole, thiadiazole, pyridine pyrimidine, pyridazine, and pyrazine;
R₂₈ is C₁₋₄ alkyl; C₂₋₄ alkyl substituted optionally with OH, NR₂₄R₂₅, C₁₋₄ alkoxy or C(═O)R₂₆; R₂₇ which can be unsubstituted or substituted optionally with OH, (CH₂)ₙ NR₂₄R₂₅, halogen, C₁₋₄ alkoxy, C₁₋₄ haloalkoxy, C(═O)R₂₆, S(═O)ₘ C₁₋₄ alkyl or SO₂ NR₂₄R₂₅; wherein m is 0-2 and n is 0-2; and
R₂₉ is C₁₋₄ alkyl; C₁₋₄ alkoxy; amino, C₁₋₃ alkylamino, of di-C₁₋₃ alkylamino.

15. The method of claim 10 wherein the carbonic anhydrase inhibitor comprises a compound having the formula 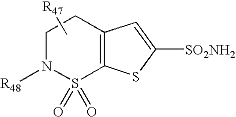
wherein:
R₄₇ is H; OH; C₁₋₆ alkoxy; C₁₋₆ alkyl unsubstituted or substituted optionally with OH, NR₄₉R₅₀, OC(═O)R₅₁ or C(O)R₅₁; NR₄₉R₅₀; OC(═O)R₅₁; C(═O)R₅₁; C₂₋₄ alkoxy substituted optionally with OH, NR₄₉R₅₀, halogen, C₁₋₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘ R₅₃ or SO₂ NR₄₉R₅₀; wherein m is 0-2 and n is 0-2; provided that when R₄₇ is OH, alkoxy, NR₄₉R₅₀ or OC(═O)R₅₁ it is attached to the 4-position and when R₄₇ is R₅₂ and is attached to the 3 position, the R₅₂ ring is attached by a carbon carbon single bond;
R₄₈ is C₂₋₈ alkyl substituted with S(═O)ₘR₅₃; C₄₋₇ alkenyl substituted with S(═O),R₅₃ wherein m is 0-2;
R₄₉ & R₅₀ are H; C₁₋₈ alkyl; C₂₋₄ alkyl substituted optionally with OH, halogen, C₁₋₄ alkoxy or C(═O)R₅₁; C₁₋₄ alkoxy; C₂₋₄ alkoxy substituted optionally with OH, halogen, C₁₋₄ alkoxy or C(═O)R₅₁; or R₄₉ and R₅₀ can be joined to form a ring of 5 or 6 atoms selected from O, S, C or N which can be unsubstituted or substituted optionally on carbon with OH, (═O), halogen, C₁₋₄ alkoxy, C(═O)R₅₁, C₁₋₆ alkyl, C-₆ alkyl substituted optionally with OH, halogen, C₁₋₄ alkoxy, C(═O)R₅₁ or on nitrogen with C₁₋₄ alkoxy, C(═O)R₅₁, S(═O)ₘR₅₃, C₁₋₆ alkyl or C₂₋₆ alkyl substituted optionally with OH, halogen, C₁₋₄ alkoxy, C(═O)R₅₁ or on sulfur by (═O)ₘ, wherein m is 0-2;
R₅₁ is C₁₋₈ alkyl; C₁₋₈ alkyl substituted optionally with OH, NR₄₉R₅₀, halogen, C₁₋₄ alkoxy or C(═O)R₅₄; C₁₋₄ alkoxy; C₂₋₄ alkoxy substituted optionally with OH, NR₄₉R₅₀, halogen or C₁₋₄ alkoxy; or NR₄₉R₅₀;
R₅₂ is a monocyclic ring system of 5 or 6 atoms composed of C, N, O or S, such as furan, thiophene, pyrrole, pyrazole, imidazole, triazole, tetrazole, oxazole, isoxazole, isothiazole, thiazole, thiadiazole, pyridine pyrimidine, pyridazine, and pyrazine;
R₅₃ is C₁₋₄ alkyl; C₃₋₅ alkenyl, C₂₋₄ alkyl substituted optionally with OH, NR₄₉R₅₀, C₁₋₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘC₁₋₄ alkyl or SO₂NR₄₉R₅₀;
m is 0-2 and n is 0-2; and
R₅₄ is C₁₋₄ alkyl; C₁₋₄ alkoxy; amino, C₁₋₃ alkylamino, or di-C₁₋₃ alkylamino.

16. The method of claim 10 wherein the carbonic anhydrase inhibitor comprises a compound having the formula 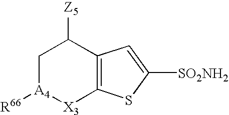
wherein:
A4 is carbon or nitrogen;
Z₅ is NHR₆₅ or OR⁶⁵;
R⁶⁵ is C₁₋₆ alkyl, either straight or branched chain;
R⁶⁶ is hydrogen, C₁₋₃ alkyl, or C₁₋₄ alkoxy-C₁₋₄ alkyl; and
X₃ is S(O)₂ or C(O)₂.

17. The method of claim 1 wherein the carbonic anhydrase inhibitor is acetazolamide.

18. The method of claim 1 wherein the carbonic anhydrase inhibitor is methazolamide.

19. The method of claim 1 wherein the carbonic anhydrase inhibitor is dichlorphenamide.

20. The method of claim 1 wherein the carbonic anhydrase inhibitor is dorzolamide.

21. The method of claim 1 wherein the carbonic anhydrase inhibitor is brinzolamide.

22. The method of claim 1 wherein the cyclooxygenase-2 selective inhibitor or pharmaceutically acceptable salt or prodrug thereof comprises a chromene compound.

23. The method of claim 22 wherein the chromene compound is a benzopyran or substituted benzopyran analog.

24. The method of claim 23 wherein the benzopyran or substituted benzopyran analog is selected from the group consisting of benzothiopyrans, dihydroquinolines and dihydronaphthalenes.

25. The method of claim 1 wherein the cyclooxygenase-2 selective inhibitor or pharmaceutically acceptable salt or prodrug thereof comprises a tricyclic compound.

26. The method of claim 25 wherein the tricyclic compound comprises a benzenesulfonamide or methylsulfonylbenzene.

27. The method of claim 1 wherein the cyclooxygenase-2 selective inhibitor or pharmaceutically acceptable salt or prodrug thereof comprises a phenyl acetic acid derivative.

28. The method of claim 1 wherein the cyclooxygenase-2 selective inhibitor comprises:

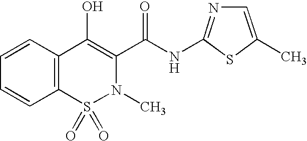

29. The method of claim 1 wherein the cyclooxygenase-2 selective inhibitor comprises:

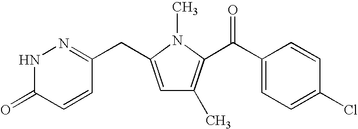

30. The method of claim 1 wherein the cyclooxygenase-2 selective inhibitor comprises a compound of the formula 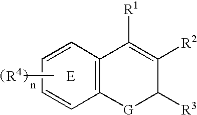
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
wherein R⁴ together with the carbon atoms to which it is attached and the remainder of ring E forms a naphthyl radical.

31. The method of claim 30 wherein:
R¹ is H;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

32. The method of claim 30 wherein:
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or wherein R⁴ together with the carbon atoms to which it is attached and the remainder of ring E forms a naphthyl radical.

33. The method of claim 30 wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

34. The method of claim 30 wherein:
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

35. The method of claim 30 wherein the cyclooxygenase-2 selective inhibitor comprises a compound of the formula 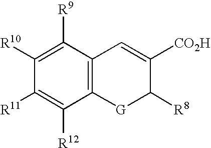
wherein:
G is oxygen or sulfur;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

36. The method of claim 1 wherein the cyclooxygenase-2 selective inhibitor comprises a compound of the formula 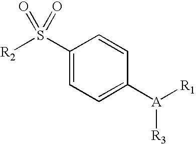
wherein:
A is selected from the group consisting of partially unsaturated or unsaturated heterocyclyl and partially unsaturated or unsaturated carbocyclic rings;
R₁ is selected from the group consisting of heterocyclyl, cycloalkyl, cycloalkenyl and aryl, wherein R₁ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R₂ is selected from the group consisting of methyl or amino; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

37. The method of claim 1 wherein the cyclooxygenase-2 selective inhibitor comprises:

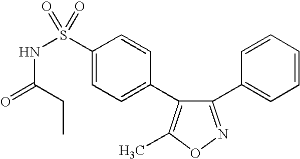

38. The method claim 1 wherein the cyclooxygenase-2 selective inhibitor comprises:

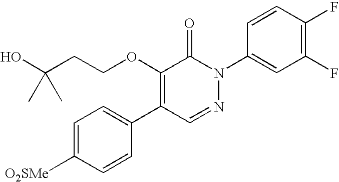

39. The method of claim 1 wherein the cyclooxygenase-2 selective inhibitor comprises 4-[4-(methyl)-sulfonyl)phenyl]-3-phenyl-2(5H)-furanone.

40. The method of claim 1 wherein the cyclooxygenase-2 selective inhibitor comprises 4-(5-methyl-3-phenyl-4-isoxazolyl).

41. The method of claim 1 wherein the cyclooxygenase-2 selective inhibitor comprises 2-(6-methylpyrid-3-yl)-3-(4-methylsulfonylphenyl)-5-chloropyridine.

42. The method of claim 1 wherein the cyclooxygenase-2 selective inhibitor comprises 4-[5-(4-methylphenyl)-3-(trifluoromethyl)-1H-pyrazol-1-yl].

43. The method of claim 1 wherein the cyclooxygenase-2 selective inhibitor comprises N-[[4-(5-methyl-3-phenyl-4-isoxazolyl)phenyl]sulfonyl].

44. The method of claim 1 wherein the cyclooxygenase-2 selective inhibitor comprises 4-[5-(3-fluoro-4-methoxyphenyl)-3-difluoromethyl)-1H-pyrazol-1-yl]benzenesulfonamide.

45. The method of claim 1 wherein the cyclooxygenase-2 selective inhibitor comprises (S)-6,8-dichloro-2-(trifluoromethyl)-2H-1-benzopyran-3-carboxylic acid.

46. The method of claim 1 wherein the cyclooxygenase-2 selective inhibitor comprises 2-(3,4-difluorophenyl)-4-(3-hydroxy-3-methylbutoxy)-5-[4-(methylsulfonyl)phenyl]-3(2H)-pyridzainone.

47. The method of claim 1 wherein the cyclooxygenase-2 selective inhibitor comprises a compound of the formula 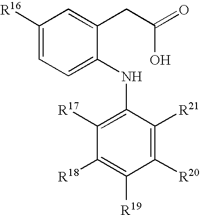
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R is ethyl and R¹⁹ is H.

48. The method of claim 47 wherein:
R¹⁶ is ethyl;
R¹⁷ and R¹⁹ are chloro;
R¹⁸ and R²⁰ are hydrogen; and
and R²¹ is methyl.

49. The method claim 1 wherein the cyclooxygenase-2 selective inhibitor comprises a compound of the formula 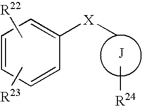
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

50. The method of claim 1 wherein the cyclooxygenase-2 selective inhibitor comprises a compound of the formula 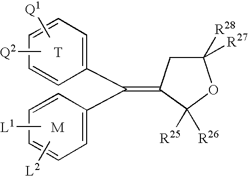
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or
R²⁷ and R²⁸ are O; or,
R₂₅, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

51. The method of claim 1 wherein the cyclooxygenase-2 selective inhibitor is selected from the group consisting of celecoxib, rofecoxib, valdecoxib, etoricoxib, parecoxib, and deracoxib.

52. The method of claim 1 wherein the neoplasia is colorectal cancer.

53. The method of claim 1 wherein the neoplasia is gastrointestinal cancer.

54. The method of claim 1 wherein the neoplasia is liver cancer.

55. The method of claim 1 wherein the neoplasia is bladder cancer

56. The method of claim 1 wherein the neoplasia is cervical cancer.

57. The method of claim 1 wherein the neoplasia is prostate cancer.

58. The method of claim 1 wherein the neoplasia is lung cancer.

59. The method of claim 1 wherein the neoplasia is breast cancer.

60. The method of claim 1 wherein the neoplasia is skin cancer.

61. The method of claim 1 wherein the neoplasia is adenomatous polyps.

62. The method of claim 1 wherein the subject is a mammal.

63. The method of claim 62 wherein the mammal is a human.

64. The method of claim 62 wherein the mammal is a companion animal.

65. The method of claim 64 wherein the companion animal is a dog or cat.

66. A composition for the treatment of neoplasia in a subject, the composition comprising administering to the subject a cyclooxygenase-2 selective inhibitor or pharmaceutically acceptable salt or prodrug thereof and a carbonic anhydrase inhibitor or pharmaceutically acceptable salt or prodrug thereof.

67. The composition of claim 66 wherein the carbonic anhydrase inhibitor comprises a benzothiazole sulfonamide.

68. The composition of claim 67 wherein the carbonic anhydrase inhibitor comprises a compound having the formula 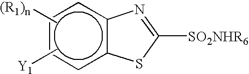
wherein:
each R₁ is hydrogen, lower alkyl, halogen, nitro, trihaloalkyl, lower alkoxy, formyl, lower alkanoyl loweralkylamino or diloweralkylamino;
R₆ is hydrogen or lower alkyl;
Y₁ is: 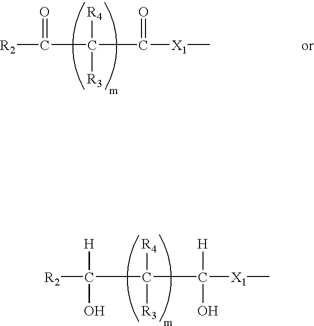
wherein:
X₁ is O or NR₅ or S;
R₂ is OR₇ or NR₇ R₈;
each R₃ and R₄ are hydrogen or lower alkyl;
R₅, R₇ and R₈ are independently hydrogen or lower alkyl;
m is an integer which is 0, 1, 2, 3, 4, 5, or 6, and
n is an integer which is 0, 1, 2, or 3.

69. The composition of claim 67 wherein the carbonic anhydrase inhibitor comprises a compound having the formula 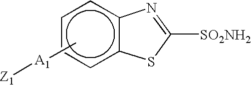
wherein:
Z₁ represents a water soluble carrier, and
A₁ is a moiety which is attached to the carbonic anhydrase inhibitor which allows it to still retain carbonic anhydrase inhibitory activity, but also form an enzymatically cleavable bond between A₁ and Z₁.

70. The composition of claim 66 wherein the carbonic anhydrase inhibitor comprises a hydroxymethazolamide.

71. The composition of claim 70 wherein the carbonic anhydrase inhibitor comprises a compound having the formula 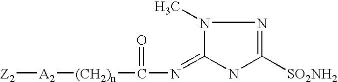
wherein:
Z₂ represents a water soluble carrier,
n is 1, 2, 3, 4, or 5; and
A₂ is a moiety which is attached to the carbonic anhydrase inhibitor which allows it to still retain carbonic anhydrase inhibitory activity, but also form an enzymatically cleavable bond between A₂ and Z₂.

72. The composition of claim 66 wherein the carbonic anhydrase inhibitor comprises a compound having the formula 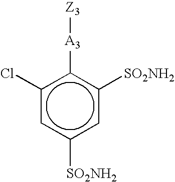
wherein:
Z₃ represents a water soluble carrier; and
A₃ is a moiety which is attached to the carbonic anhydrase inhibitor which allows it to still retain carbonic anhydrase inhibitory activity, but also form an enzymatically cleavable bond between A₃ and Z₃.

73. The composition of claim 66 wherein the carbonic anhydrase inhibitor comprises a compound having the formula 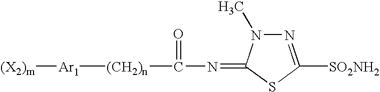
wherein:
n is an integer which is 0, 1, 2, 3,4, or 5;
X₂ is hydrogen, hydroxyl, hydroxylmethyl, 2-hydroxyethyl, or 2-hydroyethoxy;
Ar₁ is phenyl, pyridyl, or furanyl; and
m is an integer which is 0, 1, 2, 3, or 4.

74. The composition of claim 66 wherein the carbonic anhydrase inhibitor comprises a thiophene sulfonamide.

75. The composition of claim 74 wherein the carbonic anhydrase inhibitor comprises a compound having the formula 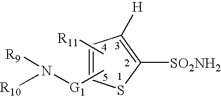
wherein:
R₉ is H, C₁₋₄ alkyl, C₂₋₄ alkyl substituted optionally with OH, halogen, C₁₋₄ alkoxy, or C(═O)R₁₅;
R₁₀ is H; C₁₋₈ alkyl; C₂₋₈ alkyl substituted with OH, NR₁₃R₁₄, halogen, C₁₋₄ alkoxy or C(═O)R₁₅; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₃ alkyl substituted with phenyl or heteroaryl which can be unsubstituted or substituted optionally with OH, (CH₂)ₙNR₁₃R₁₄, halogen, C₁₋₄ alkoxy, C₁₋₄ haloalkoxy, C(═O)R₁₅, S(═O)ₘ R₁₆ or SO₂NR₁₃R₁₄, wherein m is 0-2 and n is 0-2; C₂₋₄ alkoxy substituted optionally with NR₁₃R₁₄, halogen, C₁₋₄ alkoxy, or C(═O)R₁₅; phenyl, or heteroaryl, unsubstituted or substituted optionally with OH, (CH₂)ₙ NR₁₃R₁₄, halogen, C₁₋₄ alkoxy, C₁₋₄ haloalkoxy, C(═O)R₁₅, S(═O)ₘ R₁₆ or SO₂ NR₁₃R₁₄, wherein m is 0-2 and n is 0-2; provided that R₉ and R₁₀ cannot both be H; or R₉ and R₁₀ can be joined to form a saturated ring of 5 or 6 atoms selected from O, S, C or N which can be unsubstituted or substituted optionally on carbon with OH, NR₁₃R₁₄, halogen, C₁₋₄ alkoxy, C(═O)R₁₅, C₁₋₆ alkyl, C₁₋₆ alkyl substituted optionally with OH, NR₁₃R₁₄, halogen, C₁₋₄ alkoxy, C(═O)R₁₅ or on nitrogen with NR₁₃R₁₄, C₁₋₄ alkoxy, C(═O)R₁₅, C₁₋₆ alkyl or C₂₋₆ alkyl substituted optionally with OH, NR₁₃R₁₄, halogen, C₁₋₄ alkoxy or C(═O)R₁₅;
R₁₁ is H; halogen; C₁₋₄ alkyl; C₁₋₈ alkoxy; C₂₋₈ alkylthiol; C₂₋₈ alkoxy substituted optionally with OH, NR₁₃R₁₄, halogen, C₁₋₄ alkoxy or C(═O)R₁₅; C₁₋₄ alkyl substituted optionally with R₁₂; or R₉ and R₁₁ can be joined together with carbon atoms to form a ring of from 5 to 7 members in which said carbon atoms can be unsubstituted or substituted optionally with R₁₂;
R₁₂ is OH; C₁₋₄ alkyl unsubstituted or substituted optionally with OH, NR₁₃R₁₄, halogen, C₁₋₄ alkoxy or C(═O)R₁₅; C₁₋₄ alkoxy; C₂₋₄ alkoxy substituted optionally with OH, NR₁₃R₁₄, halogen, C₁₋₄ alkoxy or C(═O)R₁₅; NR₁₃R₁₄; phenyl, or heteroaryl, unsubstituted or substituted optionally with OH, (CH₂)ₙ NR₁₃R₁₄, halogen, C₁₋₄ alkoxy, C₁₋₄haloalkoxy, C(═O)R₁₅, S(═O)ₘ R₁₆ or SO₂NR₁₃R₁₄, wherein m is 0-2 and n is 0-2;
R₁₃ and R₁₄ are the same or different and are H; C₁₋₄ alkyl; C₂₋₄ alkyl substituted optionally with OH, halogen, C₁₋₄ alkoxy or C(═O)R₁₅; C₁₋₄ alkoxy; C₂₋₄ alkoxy substituted optionally with OH, halogen, C₁₋₄ alkoxy or C(═O)R₁₅; C₃₋₇ alkenyl unsubstituted or substituted optionally with OH, NR₁₃R₁₄, or C₁₋₄ alkoxy; C₃₋₇ alkynyl unsubstituted or substituted optionally with OH, NR₁₃R₁₄, or C₁₋₄ alkoxy; C₁₋₂ alkylC₃₋₅ cycloalkyl; or R₁₃ and R₁₄ can be joined to form a ring of 5 or 6 atoms selected from O, S, C or N which can be unsubstituted or substituted optionally on carbon with OH, (═O), halogen, C₁₋₄ alkoxy, C(═O)R₁₅, C₁₋₆ alkyl, C₁₋₆ alkyl substituted optionally with OH, halogen, C₁₋₄ alkoxy, C(═O)R₁₅ or on nitrogen with C₁₋₄ alkoxy, C(═O)R₁₅, S(═O)ₘR₁₆, C₁₋₆ alkyl or C₂₋₆ alkyl substituted optionally with OH, halogen, C₁₋₄ alkoxy, C(═O)R₁₅ or on sulfur by (═O)ₘ, wherein m is 0-2;
R₁₅ is C₁₋₈ alkyl; C₁₋₈ alkyl substituted optionally with OH, NR₁₃R₁₄, halogen, C₁₋₄ alkoxy or C(═O)R₁₇; C₁₋₄ alkoxy; C₂₋₄ alkoxy substituted optionally with OH, NR₁₃R₁₄, halogen or C₁₋₄ alkoxy; or N1₃R₁₄;
R₁₆ is C₁₋₄ alkyl; C₂₋₄ alkyl substituted optionally with OH, NR₁₃R₁₄, halogen, C₁₋₄ alkoxy or C(═O)R₁₅; and
R₁₇ is C₁₋₄ alkyl; C₁₋₄ alkoxy; amino, C₁₋₃ alkylamino, or di-C₁₋₃ alkylamino; and G₁ is C(═O) or SO₂.

76. The composition of claim 66 wherein the carbonic anhydrase inhibitor comprises a thienothiazine sulfonamide.

77. The composition of claim 76 wherein the carbonic anhydrase inhibitor comprises a compound having the formula 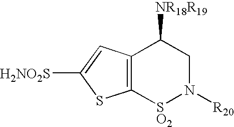
wherein:
R₁₈ and R₁₉ are H or C₁₋₄ alkyl;
R₂₀ is C₁₋₆ alkyl, CH₂(CH₂)ₙOR₂₁, where n is 1-4; and
R₂₁ is CH₃, (CH₂)ₙCH₃ where n is 1-4, or (CH₂)ₙAr₂ where Ar₂ is unsubstituted phenyl, 3-methoxyphenyl, or 4-methoxyphenyl and n is 1 or 2

78. The composition of claim 76 wherein the carbonic anhydrase inhibitor comprises a compound having the formula 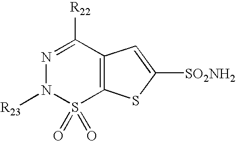
wherein:
R₂₂ is H, C₁₋₆ alkyl unsubstituted or substituted optionally with OH, C₁₋₄ alkoxy, NR₂₄R₂₅, OC(═O)R₂₆ or C(═O)R₂₆;
R₂₃ is H; C₁₋₈ alkyl; C₁₋₈ alkyl substituted with OH, NR₂₄R₂₅, halogen, C₁₋₄ alkoxy, C₂₋₄ alkoxy, C₁₋₄ alkoxy, OC(═O)R₂₆, S(═O)ₘ R₂₈, or C(═O)R₂₆; C₃₋₇ alkenyl unsubstituted or substituted optionally with OH, NR₂₄R₂₅, or C₁₋₄ alkoxy; C₃₋₇ alkynyl unsubstituted or substituted optionally with OH, NR₂₄R₂₅, or C₁₋₄ alkoxy; C₀₋₃ alkyl substituted with R₂₇ which can be unsubstituted or substituted optionally with C₁₋₃ alkyl, C₁₋₃ haloalkyl, OH, (CH₂)ₙ NR₂₄R₂₅, halogen, C₁₋₄ alkoxy, C₁₋₄ haloalkoxy, OC(═O)R₂₆, C(═O)R₂₆, S(═O)ₘ R₂₈ or SO₂ NR24R25, wherein m is 0-2 and n is 0-2;
R₂₄ and R₂₅ are independently H; C₁₋₈ alkyl; C₂₋₄ alkyl substituted optionally with OH, halogen, C₁₋₄ alkoxy or C(═O)R₂₆; OH; C₁₋₄ alkoxy; C₂₋₄ alkoxy substituted optionally with OH, halogen, C₁₋₄ alkoxy or C(═O)R₂₆; or R₂₄ and R₂₅ can be joined to form a ring of 5 or 6 atoms selected from O, S, C or N which can be unsubstituted or substituted optionally on carbon with OH, (═O), halogen, C₁₋₄ alkoxy, C(═O)R₂₆, C₁₋₆ alkyl, C₁₋₆ alkyl substituted optionally with OH, halogen, C₁₋₄ alkoxy, C(═O)R₂₆ or on nitrogen with C₁₋₄ alkoxy, C(═O)R₂₆, S(═O)ₘ R₂₈, C₁₋₆ alkyl or C₂₋₆ alkyl substituted optionally with OH, halogen, C₁₋₄ alkoxy, C(═O)R₂₆ or on sulfur by (═O)ₘ, where m is 0-2;
R₂₆ is C₁₋₈ alkyl; C₁₋₄ alkyl substituted optionally with OH, NR₂₄R₂₅, halogen, C₁₋₄ alkoxy or C(═O)R₂₉; C₁₋₄ alkoxy; C₂₋₄ alkoxy substituted optionally with OH, NR₂₄R₂₅, halogen or C₁₋₄ alkoxy; or NR₂₄R₂₅;
R₂₇, is a monocyclic ring system of 5 or 6 atoms composed of C, N, O or S, such as benzene, furan, thiophene, pyrrole, pyrazole, imidazole, triazole, tetrazole, oxazole, isoxazole, isothiazole, thiazole, thiadiazole, pyridine pyrimidine, pyridazine, and pyrazine;
R₂₈ is C₁₋₄ alkyl; C₂₋₄ alkyl substituted optionally with OH, NR₂₄R₂₅, C₁₋₄ alkoxy or C(═O)R₂₆; R₂₇ which can be unsubstituted or substituted optionally with OH, (CH₂)ₙ NR₂₄R₂₅, halogen, C₁₋₄ alkoxy, C₁₋₄ haloalkoxy, C(═O)R₂₆, S(═O)C₁₋₄ alkyl or SO₂ NR₂₄R₂₅; wherein m is 0-2 and n is 0-2; and
R₂₉ is C₁₋₄ alkyl; C₁₋₄ alkoxy; amino, C₁₋₃ alkylamino, of di-C₁₋₃ alkylamino.

79. The composition of claim 66 wherein the carbonic anhydrase inhibitor comprises a compound having the formula 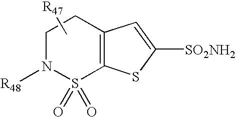
wherein:
R₄₇ is H; OH; C₁₋₆ alkoxy; C₁₋₆ alkyl unsubstituted or substituted optionally with OH, NR₄₉R₅₀, OC(═O)R₅₁ or C(═O)R₅₁; NR₄₉R₅₀; OC(═O)R₅₁; C(═O)R₅₁; C₂₋₄ alkoxy substituted optionally with OH, NR₄₉R₅₀, halogen, C₁₋₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘ R₅₃ or SO₂ NR₄₉R₅₀; wherein m is 0-2 and n is 0-2; provided that when R₄₇ is OH, alkoxy, NR₄₉R₅₀ or OC(═O)R₅₁ it is attached to the 4-position and when R₄₇ is R₅₂ and is attached to the 3 position, the R₅₂ ring is attached by a carbon carbon single bond;
R₄₈ is C₂₋₈ alkyl substituted with S(═O)ₘR₅₃; C₄₋₇ alkenyl substituted with S(═O)ₘR₅₃ wherein m is 0-2;
P₄₉ & R₅₀ are H; C₁₋₈ alkyl; C₂₋₄ alkyl substituted optionally with OH, halogen, C₁₋₄ alkoxy or C(═O)R₅₁; C₁₋₄ alkoxy; C₂₋₄ alkoxy substituted optionally with OH, halogen, C₁₋₄ alkoxy or C(═O)R₅₁; or R₄₉ and R₅₀ can be joined to form a ring of 5 or 6 atoms selected from O, S, C or N which can be unsubstituted or substituted optionally on carbon with OH, (═O), halogen, C₁₋₄ alkoxy, C(═O)R₅₁, C₁₋₆ alkyl, C₁₋₆ alkyl substituted optionally with OH, halogen, C₁₋₄ alkoxy, C(═O)R₅₁ or on nitrogen with C₁₋₄ alkoxy, C(═O)R₅₁, S(═O)ₘR₅₃, C₁₋₆ alkyl or C₂₋₆ alkyl substituted optionally with OH, halogen, C₁₋₄ alkoxy, C(═O)R₅₁ or on sulfur by (═O)ₘ, wherein m is 0-2;
R₅₁ is C₁₋₈ alkyl; C₁₋₈ alkyl substituted optionally with OH, NR₄₉R₅₀, halogen, C₁₋₄ alkoxy or C(═O)R₅₄; C₄ alkoxy; C₂₋₄ alkoxy substituted optionally with OH, NR₄₉R₅₀, halogen or C₁₋₄ alkoxy; or NR₄₉R₅₀;
R₅₂ is a monocyclic ring system of 5 or 6 atoms composed of C, N, O or S, such as furan, thiophene, pyrrole, pyrazole, imidazole, triazole, tetrazole, oxazole, isoxazole, isothiazole, thiazole, thiadiazole, pyridine pyrimidine, pyridazine, and pyrazine;
R₅₃ is C₁₋₄ alkyl; C₃₋₅ alkenyl, C₂₋₄ alkyl substituted optionally with OH, NR₄₉R₅₀, C₄ alkoxy or C(═O)R₅₁; phenyl or R₅₂ either of which can be unsubstituted or substituted optionally with OH, (CH₂)ₙNR₄₉R₅₀, halogen, C₁₋₄ alkoxy, C₁₋₄ haloalkoxy, C(═O)R₅₁, S(═O)ₘC₁₋₄ alkyl or SO₂NR₄₉R₅₀;
m is 0-2 and n is 0-2; and
R₅₄ is C₁₋₄ alkyl; C₁₋₄ alkoxy; amino, C₁₋₃ alkylamino, or di-C₁₋₃ alkylamino.

80. The composition of claim 66 wherein the carbonic anhydrase inhibitor comprises a compound having the formula 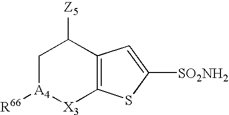
wherein:
A₄ is carbon or nitrogen;
Z₅ is NHR₆₅ or OR⁶⁵;
R⁶⁵ is C₁₋₆ alkyl, either straight or branched chain;
R⁶⁶ is hydrogen, C₁₋₃ alkyl, or C₁₋₄ alkoxy-C₁₋₄ alkyl; and
X₃ is S(O)₂ or C(O)₂.

81. The composition of claim 66 wherein the cyclooxygenase-2 selective inhibitor comprises a chromene compound.

82. The composition of claim 81 wherein the chromene compound is a benzopyran or substituted benzopyran analog.

83. The composition of claim 82 wherein the benzopyran or substituted benzopyran analog is selected from the group consisting of benzothiopyrans, dihydroquinolines and dihydronaphthalenes.

84. The composition of claim 66 wherein the cyclooxygenase-2 selective inhibitor comprises a tricyclic compound.

85. The composition of claim 84 wherein the tricyclic compound comprises a benzenesulfonamide or methylsulfonylbenzene.

86. The composition of claim 66 wherein the cyclooxygenase-2 selective inhibitor comprises a phenyl acetic acid derivative.

87. The composition of claim 66 wherein the cyclooxygenase-2 selective inhibitor comprises:

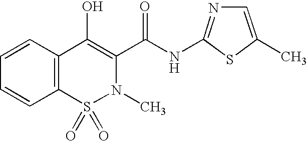

88. The composition of claim 66 wherein the cyclooxygenase-2 selective inhibitor comprises:

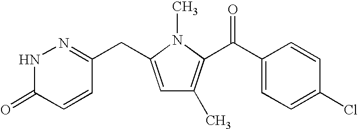

89. The composition of claim 66 wherein the cyclooxygenase-2 selective inhibitor comprises a compound of the formula 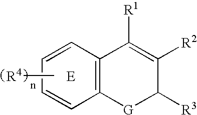
wherein:
n is an integer which is 0, 1, 2, 3 or 4;
G is O, S or NR^{a};
R^{a} is alkyl;
R¹ is selected from the group consisting of H and aryl;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl optionally substituted with one or more radicals selected from alkylthio, nitro and alkylsulfonyl; and
each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
wherein R⁴ together with the carbon atoms to which it is attached and the remainder of ring E forms a naphthyl radical.

90. The composition of claim 89 wherein:
R¹ is H;
R² is selected from the group consisting of carboxyl, aminocarbonyl, alkylsulfonylaminocarbonyl and alkoxycarbonyl;
R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

91. The composition of claim 89 wherein:
G is oxygen or sulfur;
R¹ is H;
R² is carboxyl, lower alkyl, lower aralkyl or lower alkoxycarbonyl;
R³ is lower haloalkyl, lower cycloalkyl or phenyl; and
each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or wherein R⁴ together with the carbon atoms to which it is attached and the remainder of ring E forms a naphthyl radical.

92. The composition of claim 89 wherein:
R² is carboxyl;
R³ is lower haloalkyl; and
each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

93. The composition of claim 89 wherein:
R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and
each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

94. The composition of claim 89 wherein the cyclooxygenase-2 selective inhibitor comprises a compound of the formula 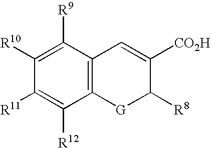
wherein:
G is oxygen or sulfur;
R⁸ is trifluoromethyl or pentafluoroethyl;
R⁹ is H, chloro, or fluoro;
R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;
R¹¹ is H, methyl, ethyl, isopropyl, tert-butyl, chloro, methoxy, diethylamino, or phenyl; and
R¹² is H, chloro, bromo, fluoro, methyl, ethyl, tert-butyl, methoxy, or phenyl.

95. The composition of claim 66 wherein the cyclooxygenase-2 selective inhibitor comprises a compound of the formula 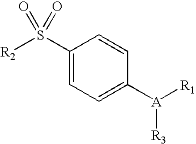
wherein:
A is selected from the group consisting of partially unsaturated or unsaturated heterocyclyl and partially unsaturated or unsaturated carbocyclic rings;
R₁ is selected from the group consisting of heterocyclyl, cycloalkyl, cycloalkenyl and aryl, wherein R₁ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
R₂ is selected from the group consisting of methyl or amino; and
R₃ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

96. The composition of claim 66 wherein the cyclooxygenase-2 selective inhibitor comprises:

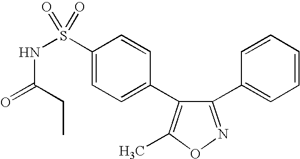

97. The composition claim 66 wherein the cyclooxygenase-2 selective inhibitor comprises:

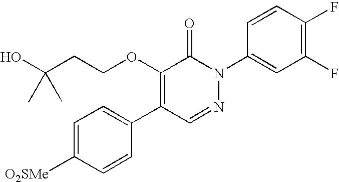

98. The composition of claim 66 wherein the cyclooxygenase-2 selective inhibitor comprises 4-[4-(methyl)-sulfonyl)phenyl]-3-phenyl-2(5H)-furanone.

99. The composition of claim 66 wherein the cyclooxygenase-2 selective inhibitor comprises 4-(5-methyl-3-phenyl-4-isoxazolyl).

100. The composition of claim 66 wherein the cyclooxygenase-2 selective inhibitor comprises 2-(6-methylpyrid-3-yl)-3-(4-methylsulfonylphenyl)-5-chloropyridine.

101. The composition of claim 66 wherein the cyclooxygenase-2 selective inhibitor comprises 4-[5-(4-methylphenyl)-3-(trifluoromethyl)-1H-pyrazol-1-yl].

102. The composition of claim 66 wherein the cyclooxygenase-2 selective inhibitor comprises N-[[4-(5-methyl-3-phenyl-4-isoxazolyl)phenyl]sulfonyl].

103. The composition of claim 66 wherein the cyclooxygenase-2 selective inhibitor comprises 4-[5-(3-fluoro-4-methoxyphenyl)-3-difluoromethyl)-1H-pyrazol-1-yl]benzenesulfonamide.

104. The composition of claim 66 wherein the cyclooxygenase 2 selective inhibitor comprises (S)-6,8-dichloro-2-(trifluoromethyl)-2H-1-benzopyran-3-carboxylic acid.

105. The composition of claim 66 wherein the cyclooxygenase-2 selective inhibitor comprises 2-(3,4-difluorophenyl)-4-(3-hydroxy-3-methylbutoxy)-5-[4-(methylsulfonyl)phenyl]-3(2H)-pyridzainone.

106. The composition of claim 66 wherein the cyclooxygenase-2 selective inhibitor comprises a compound of the formula 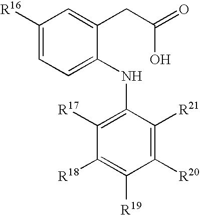
wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided that R¹⁷, R¹⁸, R¹⁹ and R²¹ are not all fluoro when R¹⁶ is ethyl and R¹⁹ is H.

107. The composition of claim 1 wherein the cyclooxygenase-2 selective inhibitor is a pharmaceutically acceptable salt or prodrug.

108. The composition of claim 107 wherein:
R¹⁶ is ethyl;
R¹⁷ and R¹⁹ are chloro;
R¹⁸ and R²⁰ are hydrogen; and
and R²¹ is methyl.

109. The composition claim 66 wherein the cyclooxygenase-2 selective inhibitor comprises a compound of the formula 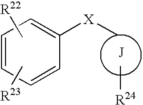
wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

110. The composition of claim 66 wherein the cyclooxygenase-2 selective inhibitor comprises a compound of the formula 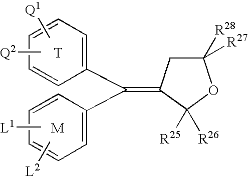
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are 0; or,
R²⁷ and R²⁸ are 0; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

111. The composition of claim 66 wherein the cyclooxygenase-2 selective inhibitor is selected from the group consisting of celecoxib, rofecoxib, valdecoxib, etoricoxib, parecoxib, and deracoxib.

112. The composition of claim 66 or 111 wherein the carbonic anhydrase inhibitor is selected from the group consisting of acetazolamide, methazolamide, dichlorphenamide, dorzolamide, and brinzolamide.

113. The composition of claim 66 wherein the carbonic anhydrase inhibitor is a geometric isomer, stereoisomer, or tautomer.

114. The composition of claim 113 wherein the carbonic anhydrase inhibitor inhibits carbonic anhydrase activity by not less than about 25% at a concentration of about 100 μM or less.

115. The composition of claim 113 wherein the carbonic anhydrase inhibitor inhibits carbonic anhydrase activity by not less than about 50% at a concentration of about 100 μM or less.

116. The composition of claim 113 wherein the carbonic anhydrase inhibitor inhibits carbonic anhydrase activity by not less than about 75% at a concentration of about 100 μM or less.

117. The composition of claim 66 wherein the cyclooxygenase-2 selective inhibitor is a geometric isomer, stereoisomer, or tautomer.

118. The composition of claim 117 wherein the cyclooxygenase-2 selective inhibitor inhibits cyclooxygenase-2 activity by not less than about 25% at a concentration of about 100 μM or less.

119. The composition of claim 117 wherein the cyclooxygenase-2 selective inhibitor inhibits cyclooxygenase-2 activity by not less than about 50% at a concentration of about 100 μM or less.

120. The composition of claim 117 wherein the cyclooxygenase-2 selective inhibitor inhibits cyclooxygenase-2 activity by not less than about 75% at a concentration of about 100 μM or less.
